SEQUENCE LISTING (2 sequences)

SEQ ID NO: 1 - RNA, 21 nt, Artificial sequence
  Synthetic polypeptide
ugugaagcgaagugcacacuu

SEQ ID NO: 2 - RNA, 19 nt, Artificial sequence
  Synthetic polypeptide
gugugcacuucgcuucaca

(19) United States
(12) Patent Application Publication
(10) Pub. No.: US 2024/0269142 A1
(43) Pub. Date: Aug. 15, 2024

(54) COMBINATION OF A TLR8 MODULATING COMPOUND AND ANTI-HBV siRNA THERAPEUTICS

(71) Applicants: GILEAD SCIENCES, INC., Foster City, CA (US); VIR BIOTECHNOLOGY, INC., San Francisco, CA (US)

(72) Inventors: Daniel J. CLOUTIER, San Francisco, CA (US); Simon P. FLETCHER, Foster City, CA (US); Phillip S. PANG, San Francisco, CA (US); Jenny Ching-Min STANTON, Acton, MA (US); Chin H. TAY, San Francisco, CA (US); Anuj GAGGAR, Redwood City, CA (US)

(21) Appl. No.: 18/559,756

(22) PCT Filed: May 12, 2022
(86) PCT No.: PCT/US2022/029022

§ 371 Date: Nov. 8, 2023

Related U.S. Application Data
(60) Provisional application No. 63/188,339, filed on May 13, 2021.
(60) Provisional application No. 63/336,709, filed on Apr. 29, 2022.

(51) Int. Cl. A61K 31/519; A61K 31/497; A61K 31/685; A61K 39/395; A61P 31/20; C12N 15/113

(52) U.S. Cl. CPC A61K 31/519; A61K 31/497; A61K 31/685; A61K 39/3955; A61P 31/20; C12N 15/1131; C12N 2310/14

(57) ABSTRACT

The present disclosure provides for the treatment and/or prevention of a hepatitis B viral infection by administering to a subject a combination therapy regimen including a toll-like receptor 8 (TLR8) modulator, a dsRNA, and a PD-1/PD-L1 inhibitor.

### CROSS-REFERENCES TO RELATED APPLICATIONS

This application claims priority to U.S. Provisional Application Nos. 63/336,709, filed Apr. 29, 2022, and 63/188,339, filed May 13, 2021, each of which is incorporated herein in its entirety for all purposes.

### BACKGROUND

The toll-like receptor (TLR) family plays a fundamental role in pathogen recognition and activation of innate immunity. Toll-like receptor 8 (TLR-8) is predominantly expressed by myeloid immune cells and activation of this receptor stimulates a broad immunological response. Agonists of TLR-8 activate myeloid dendritic cells, monocytes, monocyte-derived dendridic cells and Kupffer cells leading to the production of proinflammatory cytokines and chemokines, such as interleukin-18 (IL-18), interleukin-12 (IL-12), tumor necrosis factor-alpha (TNF-α), and interferon-gamma (IFN-γ). Such agonists also promote the increased expression of co-stimulatory molecules such as CD8+ cells, major histocompatibility complex molecules (MAIT, NK cells), and chemokine receptors. TLR8 modulating compounds include those described in U.S. Pat. No. 9,670,205.

Collectively, activation of these innate and adaptive immune responses induces an immune response and provides a therapeutic benefit in various conditions involving autoimmunity, inflammation, allergy, asthma, graft rejection, graft versus host disease (GvHD), infection, cancer, and immunodeficiency. For example, with respect to hepatitis B, activation of TLR8 on professional antigen presenting cells (pAPCs) and other intrahepatic immune cells is associated with induction of IL-12 and proinflammatory cytokines, which is expected to augment HBV-specific T cell responses, activate intrahepatic NK cells and drive reconstitution of antiviral immunity. See e.g. Wille-Reece, U. et al. J Exp Med 203, 1249-1258 (2006); Peng, G. et al., Science 309, 1380-1384 (2005); Jo, J. et al., PLoS Pathogens 10, e1004210 (2014) and Watashi, K. et al., J Biol Chem 288, 31715-31727 (2013).

There remains a need for new agents and therapies capable of assisting in the activation of the latent HBV-infected cells to enhance the activity of antiretroviral therapies and immune responses.

### BRIEF SUMMARY OF THE DISCLOSURE

In some embodiments, provided herein is a method of treating and/or preventing a hepatitis B viral infection in a subject in need thereof, comprising administering to the subject a therapeutically effective amount of a combination therapy regimen comprising a compound of Formula (I):

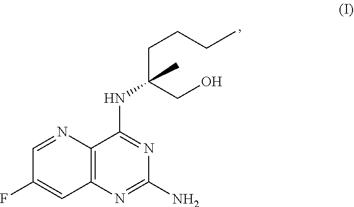

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and a PD-1/PD-L1 inhibitor, or a pharmaceutically acceptable salt thereof, thereby treating and/or preventing the hepatitis B viral infection in the subject.

### DETAILED DESCRIPTION OF THE DISCLOSURE

### I. General

The present disclosure describes a method of treating and/or preventing a heptatis B viral infection in a subject in need thereof comprising administering a therapeutically effective amount of a combination of a toll-like receptor 8 (TLR8) modulator, an anti-HBV siRNA or dsRNA, and a programmed cell death protein 1 (PD-1)/programmed death-ligand 1 (PD-L1) inhibitor. The methods of the present disclosure may also include other additional therapeutic agents.

### II. Definitions

Unless specifically indicated otherwise, all technical and scientific terms used herein have the same meaning as commonly understood by those of ordinary skill in the art to which this disclosure belongs. In addition, any method or material similar or equivalent to a method or material described herein can be used in the practice of the present disclosure. For purposes of the present disclosure, the following terms are defined.

“A,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

Reference to “about” a value or parameter herein includes (and describes) embodiments that are directed to that value or parameter per se. In certain embodiments, the term “about” includes the indicated amount +10%. In other embodiments, the term “about” includes the indicated amount +5%. In certain other embodiments, the term “about” includes the indicated amount +1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

As used herein, “treat”, “treatment” or “treating” is an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival.

As used herein, “prevent”, “prevention” or “preventing” refers to a regimen that protects against the onset of the disease or disorder such that the clinical symptoms of the disease do not develop. Thus, “prevention” relates to administration of a therapy (e.g., administration of a therapeutic substance) to a subject before signs of the disease are detectable in the subject (e.g., administration of a therapeutic substance to a subject in the absence of detectable infectious agent (e.g., virus) in the subject). The subject may be an individual at risk of developing the disease or disorder, such as an individual who has one or more risk factors known to be associated with development or onset of the disease or disorder. Thus, in certain embodiments, the term “preventing HBV infection” or “preventing hepatitis B viral infection” refers to administering to a subject who does not have a detectable HBV infection an anti-HBV therapeutic substance. It is understood that the subject for anti-HBV preventative therapy may be an individual at risk of contracting the HBV virus.

As used herein, “hepatitis B viral infection”, or HBV, refers to a viral infection that affects the liver, and is caused by the hepatitis B virus.

As used herein, “hepatitis D viral infection”, or HDV, refers to a viral infection that affects the liver, and is caused by the hepatitis D virus.

As used herein, “subject” refers to animals such as mammals, including, but not limited to, primates (e.g., humans), cows, sheep, goats, horses, dogs, cats, rabbits, rats, mice and the like. In certain embodiments, the subject is a human.

As used herein, “administering” refers to oral administration, administration as a suppository, topical contact, parenteral, intravenous, subcutaneous, intraperitoneal, intramuscular, intralesional, intranasal or subcutaneous administration, intrathecal administration, or the implantation of a slow-release device e.g., a mini-osmotic pump, to the subject.

As used herein, the term “therapeutically effective amount” or “effective amount” refers to an amount that is effective to elicit the desired biological or medical response, including the amount of a compound that, when administered to a subject for treating a disease, is sufficient to effect such treatment for the disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

As used herein, “combination therapy regimen” refers to administering two or more therapeutic agents to a subject in need thereof. In some non-limiting examples, the two or more therapeutic agents are administered at different times. In some examples, the two or more therapeutic agents are administered at the same time. The combination therapy regimen is administered by any method described herein.

Administration can also include co-administration such that two or more therapeutic agents are delivered together during the course of the treatment. In one embodiment, two or more therapeutic agents may be co-formulated into a single dosage form or “combined dosage unit”, or formulated separately and subsequently combined into a combined dosage unit, as is typically for intravenous administration or oral administration as a mono or bilayer tablet or capsule.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for human pharmaceutical use.

As used herein, “pharmaceutically acceptable salts” refers to salts derived from an appropriate base, such as an alkali metal (for example, sodium, potassium), an alkaline earth metal (for example, magnesium), ammonium and NX₄⁺ (wherein X is C₁-C₄ alkyl). Also included are base addition salts, such as sodium or potassium salts. Pharmaceutically acceptable salts are non-toxic salts of a free base form of a compound that possesses the desired pharmacological activity of the free base. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington: The Science and Practice of Pharmacy, 21^{st} Edition, Lippincott Wiliams and Wilkins, Philadelphia, Pa., 2006.

As used herein, “double stranded ribonucleic acid (dsRNA)” refers to a complex of ribonucleic acid molecules, having a duplex structure comprising two anti-parallel and substantially complementary nucleic acid strands, referred to as having “sense” and “antisense” orientations with respect to a target RNA, i.e., an HBV gene. In some embodiments of the present disclosure, a dsRNA triggers the degradation of a target RNA, e.g., an mRNA, through a post-transcriptional gene-silencing mechanism referred to herein as RNA interference or RNAi.

As used herein, “PD-1/PD-L1 inhibitors” refers to checkpoint inhibitors that block the activity of a programmed cell death protein 1 (PD-1)/programmed death-ligand 1 (PD-L1) immune checkpoint proteins. The PD-1/PD-L1 inhibitors can act to inhibit associating programed death-ligand 1 (PD-L1) with its receptor, programed cell death protein 1 (PD-1).

As used herein, “fasting” refers to not eating and/or drinking for a specific amount of time. In a non-limiting example, fasting may refer to a subject who has not eaten and/or consumed liquid from 8 to 24 hours since the last meal. In some examples, fasting may refer to a subject who has not eaten and/or consumed liquid from 8 to 12 hours since the last meal. In some examples, the subject may not have eaten and/or consumed liquid for between 6 to 12 hours since the last meal.

The term “viral load” refers to the quantity of a virus in an amount of fluid, which may be measured volumetrically. Viral load can be expressed as viral or infectious particles per mL. Viral load can be expressed as international units per milliliter (IU/mL). A higher viral load may correlate with the severity of an active viral infection. Tests for determining viral load may include, but are not limited to reverse transcription-polymerase chain reaction (RT-PCR) tests, branched DNA (bDNA) tests, Qualitative Transcription-Mediated Amplification Assays, and nucleic acid sequence-based amplification (NASBA) tests.

### III. Methods of Treatment

The present disclosure describes combinations of a TLR8-modulating compound, an anti-HBV siRNA or dsRNA therapeutic, and a PD-1/PD-L1 inhibitor.

In some embodiments, provided herein is a method of treating and/or preventing a hepatitis B viral infection in a subject in need thereof, comprising administering to the subject a therapeutically effective amount of a combination therapy regimen comprising a compound of Formula (I):

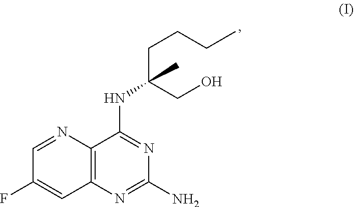

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and a PD-1/PD-L1 inhibitor, or a pharmaceutically acceptable salt thereof, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

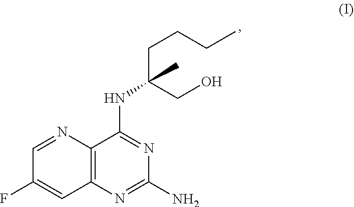

or a pharmaceutically acceptable salt thereof, a double stranded ribonucleic acid (dsRNA) comprising SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2-O-methylcytidine-3′-phosphate, each g is 2′-O-methylguanosine-3-phosphate, each u is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and a PD-1/PD-L1 inhibitor, or a pharmaceutically acceptable salt thereof, thereby treating and/or preventing the hepatitis B viral infection in the subject.

In some embodiments, the method comprises treating or preventing the hepatitis B viral infection in the subject in need thereof. In some embodiments, the method comprises treating the hepatitis B viral infection in the subject in need thereof. In some embodiments, provided herein is a method of treating a hepatitis B viral infection in a subject in need thereof, comprising administering to the subject a therapeutically effective amount of a combination therapy regimen comprising a compound of Formula (I):

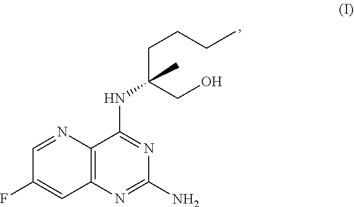

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and a PD-1/PD-L1 inhibitor, or a pharmaceutically acceptable salt thereof, thereby treating and/or preventing the hepatitis B viral infection in the subject.

In some embodiments, provided herein is a method of treating a hepatitis B viral infection in a subject in need thereof, comprising administering to the subject a therapeutically effective amount of a combination therapy regimen comprising a compound of Formula (I):

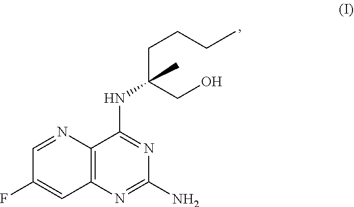

or a pharmaceutically acceptable salt thereof, a double stranded ribonucleic acid (dsRNA) comprising SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3′-phosphate, each u is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and a PD-1/PD-L1 inhibitor, or a pharmaceutically acceptable salt thereof, thereby treating and/or preventing the hepatitis B viral infection in the subject.

In some embodiments, the method comprises preventing the hepatitis B viral infection in the subject in need thereof. In some embodiments, provided herein is a method of preventing a hepatitis B viral infection in a subject in need thereof, comprising administering to the subject a therapeutically effective amount of a combination therapy regimen comprising a compound of Formula (I):

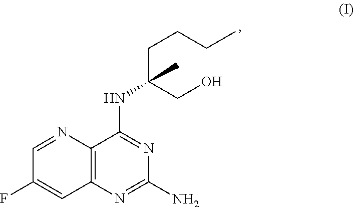

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and a PD-1/PD-L1 inhibitor, or a pharmaceutically acceptable salt thereof, thereby treating and/or preventing the hepatitis B viral infection in the subject.

In some embodiments, provided herein is a method of preventing a hepatitis B viral infection in a subject in need thereof, comprising administering to the subject a therapeutically effective amount of a combination therapy regimen comprising a compound of Formula (I):

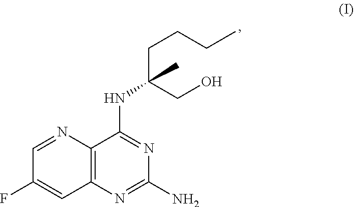

or a pharmaceutically acceptable salt thereof, a double stranded ribonucleic acid (dsRNA) comprising SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3′-phosphate, each u is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and a PD-1/PD-L1 inhibitor, or a pharmaceutically acceptable salt thereof, thereby treating and/or preventing the hepatitis B viral infection in the subject.

The combination therapy regimens of the present disclosure can also be used to treat and/or prevent a hepatitis D viral infection in a subject in need thereof. In some embodiments, provided herein is a method of treating and/or preventing a hepatitis D viral infection in a subject in need thereof, comprising administering to the subject a therapeutically effective amount of a combination therapy regimen comprising a compound of Formula (I):

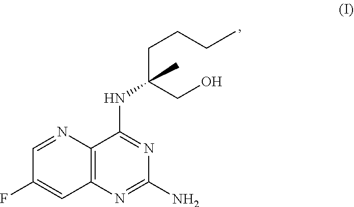

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and a PD-1/PD-L1 inhibitor, or a pharmaceutically acceptable salt thereof, thereby treating and/or preventing the hepatitis B viral infection in the subject.

In some embodiments, provided herein is a method of treating and/or preventing a hepatitis D viral infection in a subject in need thereof, comprising administering to the subject a therapeutically effective amount of a combination therapy regimen comprising a compound of Formula (I):

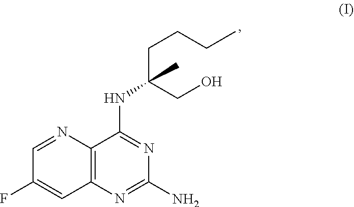

or a pharmaceutically acceptable salt thereof, a double stranded ribonucleic acid (dsRNA) comprising SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3-phosphate, each c is 2′-O-methylcytidine-3′-phosphate, each g is 2′-O-methylguanosine-3′ phosphate, each u is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and a PD-1/PD-L1 inhibitor, or a pharmaceutically acceptable salt thereof, thereby treating and/or preventing the hepatitis B viral infection in the subject.

### A. Combination Therapy Regimen

The combination therapy regimens of the present disclosure can include a variety of toll-like receptor 8 (TLR8) modulators. In some embodiments, the compound of Formula (I) is a toll-like receptor 8 (TLR8) modulator. Examples of TLR-8 modulators include, but are not limited to, GS-9688, also referred to as selgantolimod or (R)-2-((2-amino-7-fluoropyrido[3,2-d]pyrimidin-4-yl)amino)-2-methylhexan-1-ol, and has the following structure:

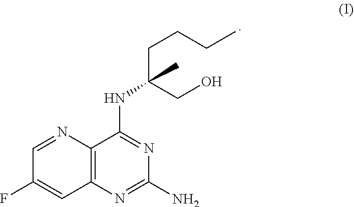

The compound of Formula (I) is described in Example 98 of U.S. Pat. No. 9,670,205 and WO 2016/141092. Other forms of the compound of Formula (I) are described in WO 2020/214663 and WO 2020/214652.

In some embodiments, TLR8 modulators that can be administered include, but are not limited to, E-6887, IMO-4200, IMO-8400, IMO-9200, MCT-465, MEDI-9197, motolimod, resiquimod, selgantolimod (GS-9688), HRS-9950, VTX-1463, VTX-763, 3M-051, 3M-052, and the compounds disclosed in U.S. Pat. No. 9,670,205 (Gilead Sciences), U.S. Pat. No. 10,285,990 (Gilead Sciences), US2019/0282576 (Gilead Sciences), WO2016/141092 (Gilead Sciences), US2016/0289229 (Gilead Sciences), US2014/0045849 (Janssen), US2014/0073642 (Janssen), WO2014/056953 (Janssen), WO2014/076221 (Janssen), WO2014/128189 (Janssen), US2014/0350031 (Janssen), WO2014/023813 (Janssen), US2008/0234251 (Array Biopharma), US2008/0306050 (Array Biopharma), US2010/0029585 (Ventirx Pharma), US2011/0092485 (Ventirx Pharma), US2011/0118235 (Ventirx Pharma), US2012/0082658 (Ventirx Pharma), US2012/0219615 (Ventirx Pharma), US2014/0066432 (Ventirx Pharma), US2014/0088085 (Ventirx Pharma), US2014/0275167 (Novira Therapeutics), US2013/0251673 (Novira Therapeutics), U.S. Pat. No. 9,670,205 (Gilead Sciences, Inc.), US2016/0289229 (Gilead Sciences, Inc.), WO2017/048727 (Gilead Sciences, Inc.), US2018/0065938 (Gilead Sciences, Inc.), and US2018/0086755 (Gilead Sciences, Inc.). In some embodiments, the compound of Formula (I) is selgantolimod (SLGN).

In some embodiments, the compound of Formula (I) has the structure:

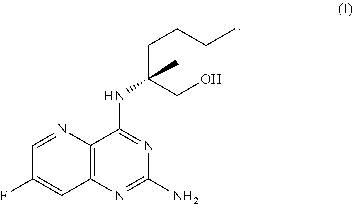

The combination therapy regimens of the present disclosure can include a variety of hepatitis B virus double-stranded RNA (dsRNA) for inhibiting expression of the hepatitis B virus. Representative dsRNA useful in the combination therapy regimen of the present disclosure are described in WO 2020/036862. Other dsRNA useful for inhibiting expression of the hepatitis B virus are known to one of skill in the art.

In some embodiments, the dsRNA is a small interfering RNA (siRNA). In some embodiments, the dsRNA can include, but is not limited to, the dsRNA described in WO2020/036862. In some embodiments, the dsRNA is chemically modified to enhance stability or other beneficial characteristics.

In some embodiments, the dsRNA further comprises a ligand. The ligand can be conjugated to the 3′ end of the sense strand of the dsRNA. The ligand can be an N-acetylgalactosamine (GalNAc) derivative. The ligand can be:

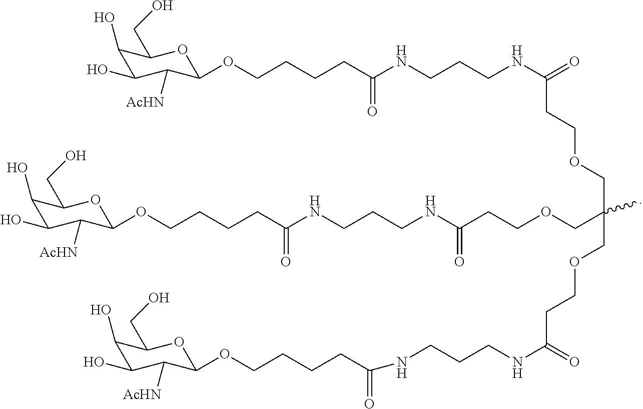 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, the dsRNA is conjugated to the ligand as shown in the following schematic:

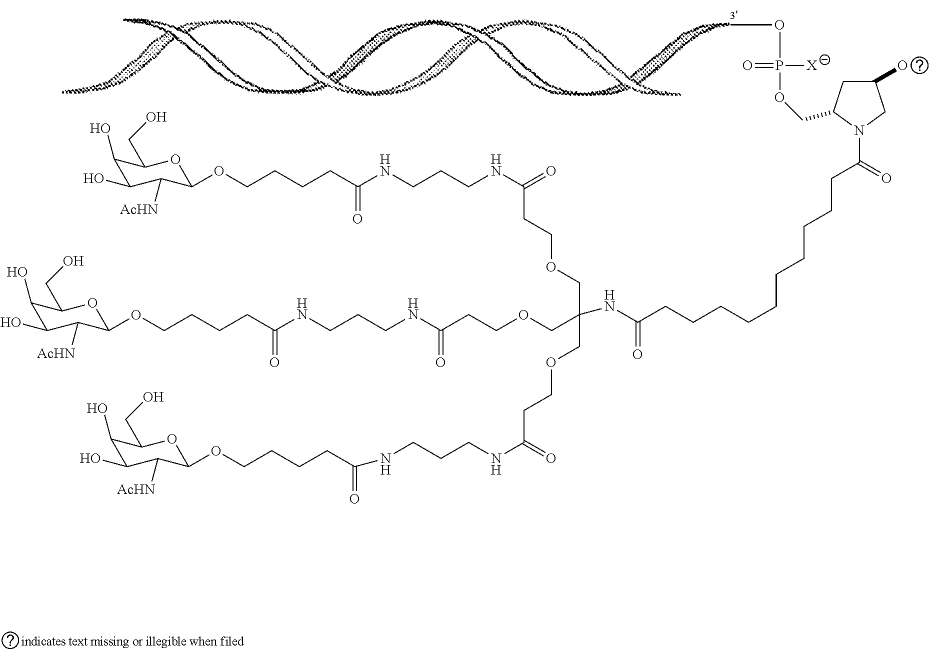

wherein X is O or S. In some embodiments, X is O.

In some embodiments, the dsRNA is modified to include one or more adenosine-glycol nucleic acid (“GNA”). The term “GNA” refers to glycol nucleic acid which is a polymer similar to DNA or RNA but differing in the composition of its “backbone” in that it is composed of repeating glycerol units linked by phosphodiester bonds:

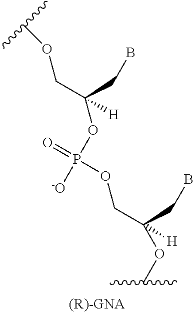

wherein each B is independently a nucleobase. A description of adenosine-GNA can be found, for example, in Zhang, et al. (JACS 127(12):4174-75 (2005)).

In some embodiments, the dsRNA comprises SEQ ID NO.: 1 and SEQ ID NO.: 2, wherein SEQ ID NO.: 1 is 5′-usGfsuga(Agn)gCfGfaaguGfcAfcacsusu-3′ and SEQ ID NO.: 2 is 5′-gsusguGfcAfCfUfucgcuucacaL96-3′, wherein each a is 2-O-methyladenosine-3′-phosphate, each c is 2′-O-methylcytidine-3′-phosphate, each g is 2′-O-methylguanosine-3′-phosphate, each u is 2′-O-nethyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-anidodecanoyl)]-4-hydroxyprolinol.

In some embodiments, the dsRNA includes an antisense strand and a sense strand. In some embodiments, the antisense strand is SEQ ID NO.: 1. The antisense strand of SEQ ID NO.:1 corresponds to SEQ ID NO:16 of WO2020/036862. In some embodiments, the sense strand is SEQ ID NO.:2. The sense strand of SEQ ID NO.:2 corresponds to SEQ ID NO:29 of WO2020/036862 having an N-acetylgalactosamine moiety N-[tris(GalNAc-alkyl)-amidodecanoyl)]-4-hydroxyprolinol (also known as (Hyp-(GalNAc-alkyl)3) or L96) covalently linked to the 3′ end.

The combination therapy regimens of the present disclosure can include a variety of programmed cell death protein 1 (PD-1)/programmed death-ligand 1 (PD-L1) inhibitors.

In some embodiments, the PD-1/PD-L1 inhibitor is nivolumab, pembrolizumab, pidilzumab, BGB-108, SHR-1210, PDR-001, PF-06801591, IBI-308, GB-226, STI-1110, or mDX-400, or a pharmaceutically acceptable salt thereof. In some embodiments, the PD-1/PD-L1 inhibitor is nivolumab or a pharmaceutically acceptable salt thereof. In some embodiments, the PD-1/PD-L1 inhibitor is nivolumab. Nivolumab (Opdivo) is a programmed death receptor-1 (PD-1) blocking antibody, a IgG4 kappa immunoglobulin having a calculated molecular mass of about 146 kDa.

In some embodiments, the PD-1/PD-L1 inhibitor is pembrolizumab, or a pharmaceutically acceptable salt thereof. In some embodiments, the PD-1/PD-L1 inhibitor is pidilzumab, or a pharmaceutically acceptable salt thereof. In some embodiments, the PD-1/PD-L1 inhibitor is BGB-108, or a pharmaceutically acceptable salt thereof. In some embodiments, the PD-1/PD-L1 inhibitor is SHR-1210, or a pharmaceutically acceptable salt thereof. In some embodiments, the PD-1/PD-L1 inhibitor is PDR-001, or a pharmaceutically acceptable salt thereof. In some embodiments, the PD-1/PD-L1 inhibitor is PF-06801591, or a pharmaceutically acceptable salt thereof. In some embodiments, the PD-1/PD-L1 inhibitor is IBI-308, or a pharmaceutically acceptable salt thereof. In some embodiments, the PD-1/PD-L1 inhibitor is GB-226, or a pharmaceutically acceptable salt thereof. In some embodiments, the PD-1/PD-L1 inhibitor is STI-1110, or a pharmaceutically acceptable salt thereof. In some embodiments, the PD-1/PD-L1 inhibitor is mDX-400, or a pharmaceutically acceptable salt thereof.

In some embodiments, the PD-1/PD-L1 inhibitor is GS-4224, atezolizumab, avelumab, zimberelimab, AMP-224, MEDI-0680, RG-7446, GX-P2, durvalumab, KY-1003, KD-033, MSB-0010718C, TSR-042, ALN-PDL, STI-A1014, CX-072, or BMS-936559, or a pharmaceutically acceptable salt thereof. In some embodiments, the PD-1/PD-L1 inhibitor is GS-4224, or a pharmaceutically acceptable salt thereof. In some embodiments, the PD-1/PD-L1 inhibitor is atezolizumab, or a pharmaceutically acceptable salt thereof. In some embodiments, the PD-1/PD-L1 inhibitor is avelumab, or a pharmaceutically acceptable salt thereof. In some embodiments, the PD-1/PD-L1 inhibitor is zimberelimab, or a pharmaceutically acceptable salt thereof. In some embodiments, the PD-1/PD-L1 inhibitor is AMP-224, or a pharmaceutically acceptable salt thereof. In some embodiments, the PD-1/PD-L1 inhibitor is MEDI-0680, or a pharmaceutically acceptable salt thereof. In some embodiments, the PD-1/PD-L1 inhibitor is RG-7446, or a pharmaceutically acceptable salt thereof. In some embodiments, the PD-1/PD-L1 inhibitor is GX-P2, or a pharmaceutically acceptable salt thereof. In some embodiments, the PD-1/PD-L1 inhibitor is durvalumab, or a pharmaceutically acceptable salt thereof. In some embodiments, the PD-1/PD-L1 inhibitor is KY-1003, or a pharmaceutically acceptable salt thereof. In some embodiments, the PD-1/PD-L1 inhibitor is KD-033, or a pharmaceutically acceptable salt thereof. In some embodiments, the PD-1/PD-L1 inhibitor is MSB-0010718C, or a pharmaceutically acceptable salt thereof. In some embodiments, the PD-1/PD-L1 inhibitor is TSR-042, or a pharmaceutically acceptable salt thereof. In some embodiments, the PD-1/PD-L1 inhibitor is ALN-PDL, or a pharmaceutically acceptable salt thereof. In some embodiments, the PD-1/PD-L1 inhibitor is STI-A1014, or a pharmaceutically acceptable salt thereof. In some embodiments, the PD-1/PD-L1 inhibitor is CX-072, or a pharmaceutically acceptable salt thereof. In some embodiments, the PD-1/PD-L1 inhibitor is BMS-936559, or a pharmaceutically acceptable salt thereof.

Additional PD-1/PD-L1 inhibitors include, but are not limited to, compounds described in U.S. Pat. Nos. 10,710,986 and 10,774,071. In some embodiments, the PD-1/PD-L1 inhibitor is:

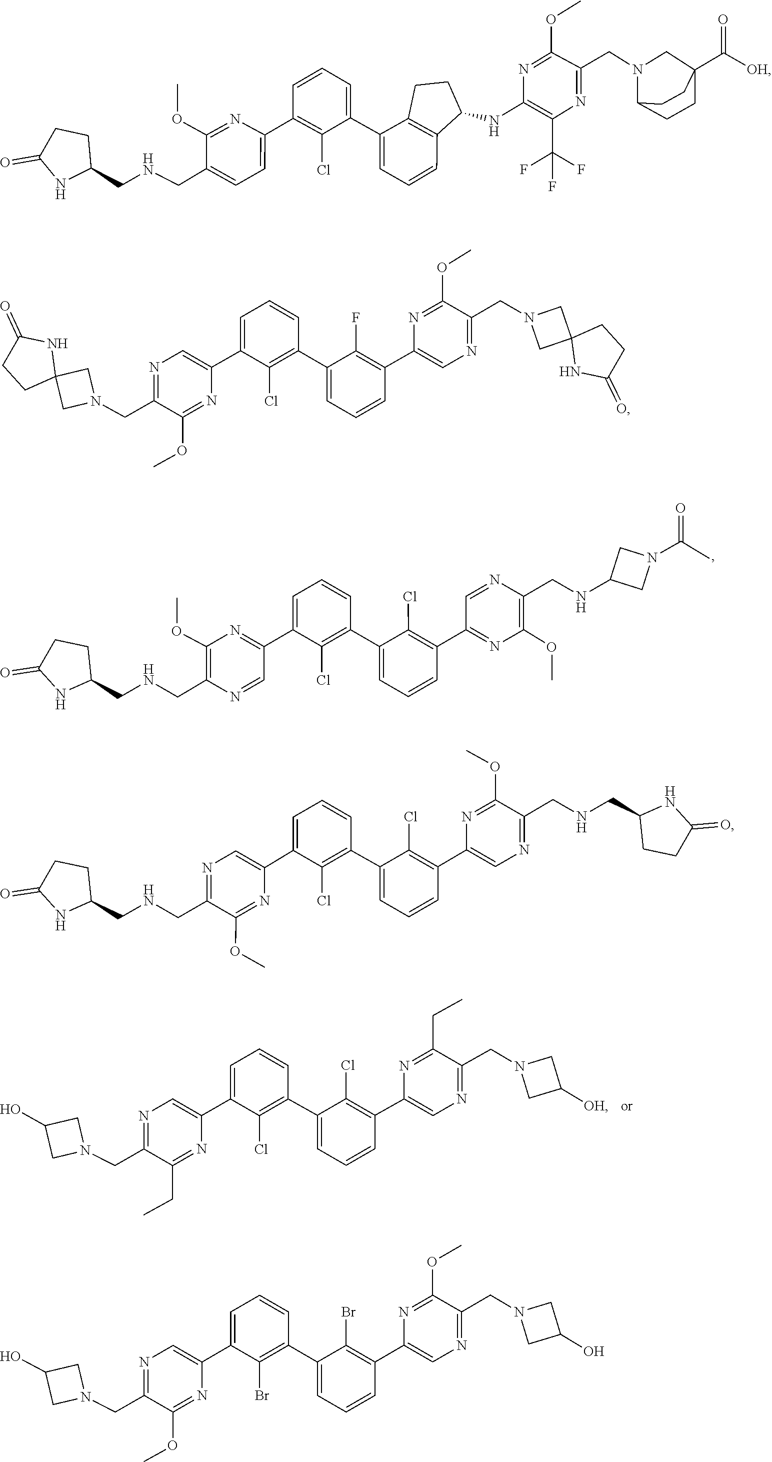

or a pharmaceutically acceptable salt thereof.

In some embodiments, the PD-1/PD-L1 inhibitor is:

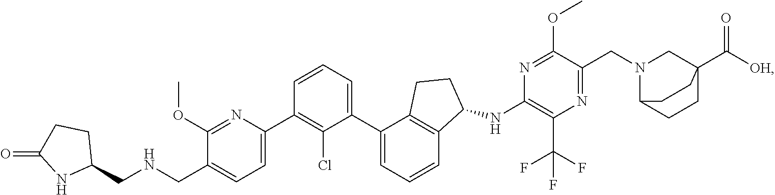 [SMILES: COc1nc(-c2cccc(-c3cccc4c3CC[C@@H]4Nc3nc(OC)c(CN4CC5(C(=O)O)CCC4CC5)nc3C(F)(F)F)c2Cl)ccc1CNC[C@@H]1CCC(=O)N1]

or a pharmaceutically acceptable salt thereof.

In some embodiments, the PD-1/PD-L1 inhibitor is:

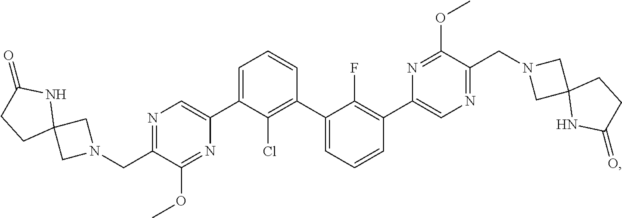 [SMILES: COc1nc(-c2cccc(-c3cccc(-c4cnc(CN5CC6(CCC(=O)N6)C5)c(OC)n4)c3Cl)c2F)cnc1CN1CC2(CCC(=O)N2)C1]

or a pharmaceutically acceptable salt thereof.

In some embodiments, the PD-1/PD-L1 inhibitor is:

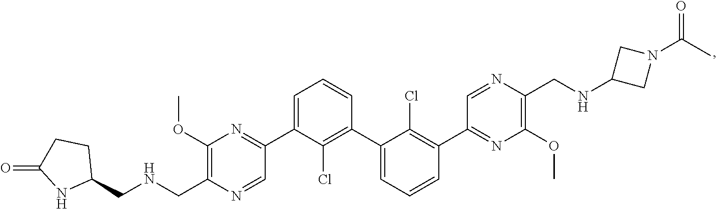 [SMILES: COc1nc(-c2cccc(-c3cccc(-c4cnc(CNC5CN(C(C)=O)C5)c(OC)n4)c3Cl)c2Cl)cnc1CNC[C@@H]1CCC(=O)N1]

or a pharmaceutically acceptable salt thereof.

In some embodiments, the PD-1/PD-L1 inhibitor is:

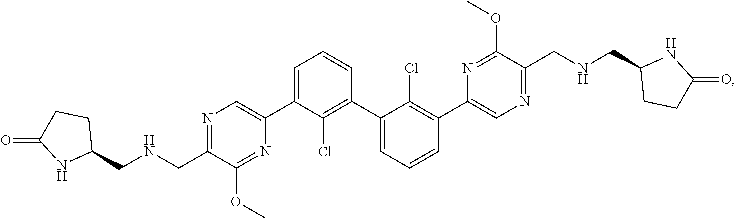 [SMILES: COc1nc(-c2cccc(-c3cccc(-c4cnc(CNC[C@@H]5CCC(=O)N5)c(OC)n4)c3Cl)c2Cl)cnc1CNC[C@@H]1CCC(=O)N1]

or a pharmaceutically acceptable salt thereof.

In some embodiments, the PD-1/PD-L1 inhibitor is:

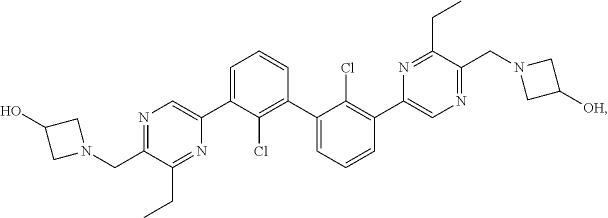 [SMILES: CCc1nc(-c2cccc(-c3cccc(-c4cnc(CN5CC(O)C5)c(CC)n4)c3Cl)c2Cl)cnc1CN1CC(O)C1]

or a pharmaceutically acceptable salt thereof.

In some embodiments, the PD-1/PD-L1 inhibitor is:

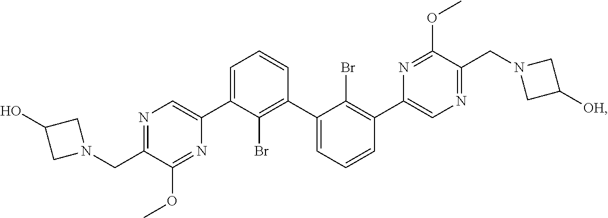 [SMILES: COc1nc(-c2cccc(-c3cccc(-c4cnc(CN5CC(O)C5)c(OC)n4)c3Br)c2Br)cnc1CN1CC(O)C1]

or a pharmaceutically acceptable salt thereof.

In some embodiments, provided herein is a method of treating and/or preventing a hepatitis B viral infection in a subject in need thereof, comprising administering to the subject a therapeutically effective amount of a combination therapy regimen comprising a compound of Formula (I):

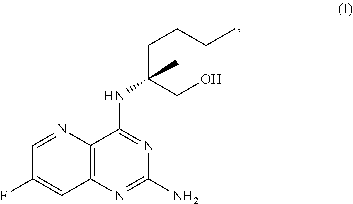

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and nivolumab, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

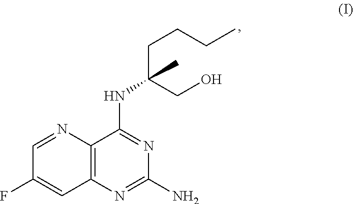

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3-phosphate, each c is 2′-O-methylcytidine-3′-phosphate, each g is 2′-O-methylguanosine-3′ phosphate, each u is 2′-O-nethyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and nivolumab, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

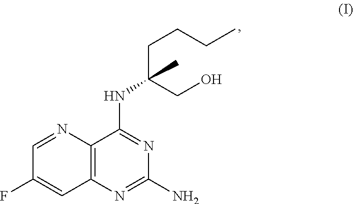

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and pembrolizumab, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

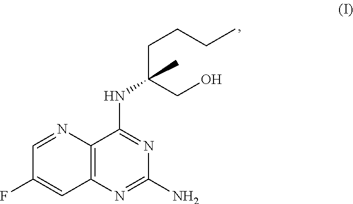

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3-phosphate, each u is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and pembrolizumab, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

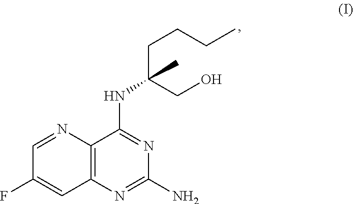

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and pidilzumab, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

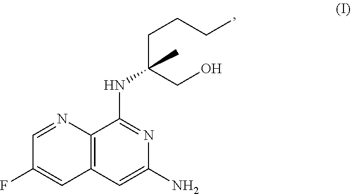

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3′-phosphate, each u is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and pidilzumab, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

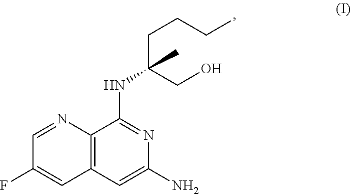

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and atezolizumab, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

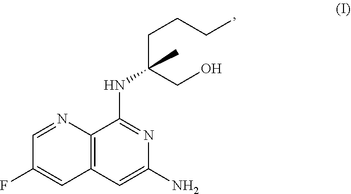

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3′-phosphate, each u is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and atezolizumab, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

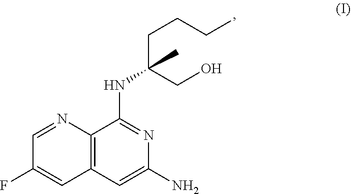

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and avelumab, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

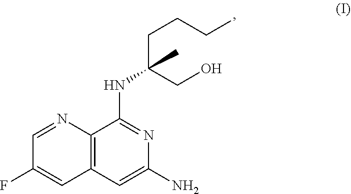

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3′-phosphate, each a is 2′-O-methyluridine-3′-phosphinate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and avelumab, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

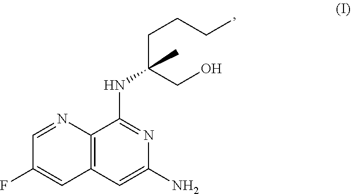

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and zimberelimab, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

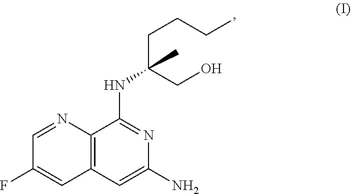

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3′ phosphate, each u is 2′-O-methyluridine-3′-phosphate, each Af is 2′-fluoroadenosine-3-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and zimberelimab, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

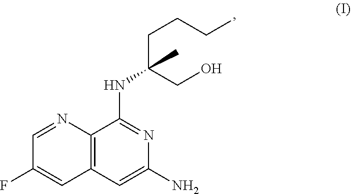

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and durvalumab, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

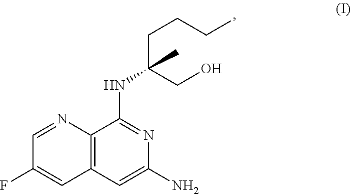

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3′-phosphate, each a is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and durvalumab, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

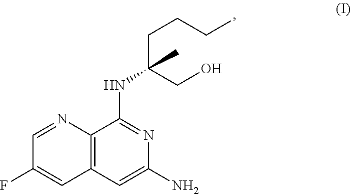

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and the compound:

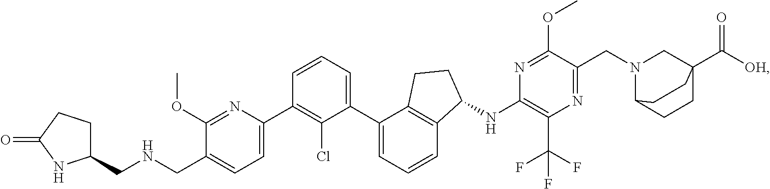 [SMILES: COc1nc(-c2cccc(-c3cccc4c3CC[C@@H]4Nc3nc(OC)c(CN4CC5(C(=O)O)CCC4CC5)nc3C(F)(F)F)c2Cl)ccc1CNC[C@@H]1CCC(=O)N1]

or a pharmaceutically acceptable salt thereof, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

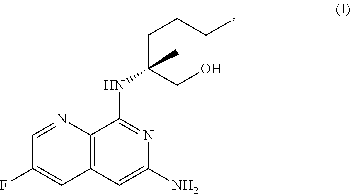

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3′-phosphate, each u is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and the compound:

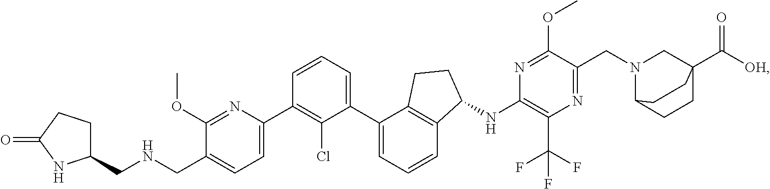 [SMILES: COc1nc(-c2cccc(-c3cccc4c3CC[C@@H]4Nc3nc(OC)c(CN4CC5(C(=O)O)CCC4CC5)nc3C(F)(F)F)c2Cl)ccc1CNC[C@@H]1CCC(=O)N1]

or a pharmaceutically acceptable salt thereof, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

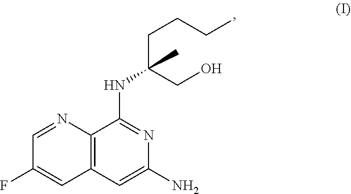

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and the compound:

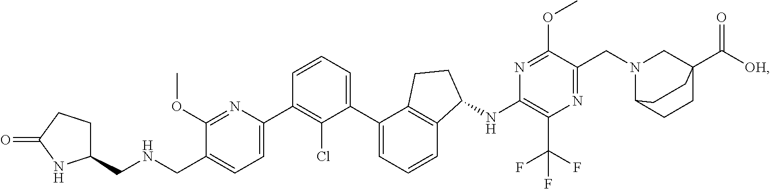 [SMILES: COc1nc(-c2cccc(-c3cccc4c3CC[C@@H]4Nc3nc(OC)c(CN4CC5(C(=O)O)CCC4CC5)nc3C(F)(F)F)c2Cl)ccc1CNC[C@@H]1CCC(=O)N1]

or a pharmaceutically acceptable salt thereof, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

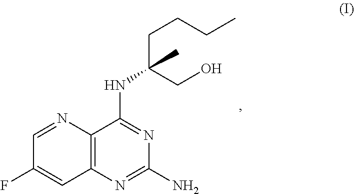

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3′-phosphate, each u is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and the compound:

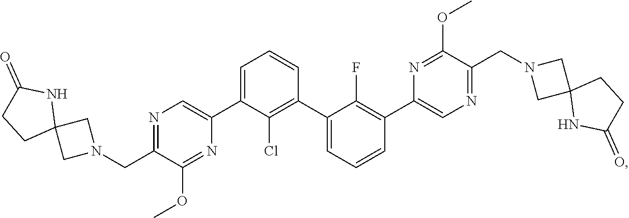 [SMILES: COc1nc(-c2cccc(-c3cccc(-c4cnc(CN5CC6(CCC(=O)N6)C5)c(OC)n4)c3Cl)c2F)cnc1CN1CC2(CCC(=O)N2)C1]

or a pharmaceutically acceptable salt thereof, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

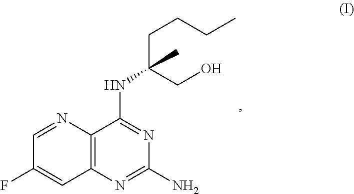

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and the compound:

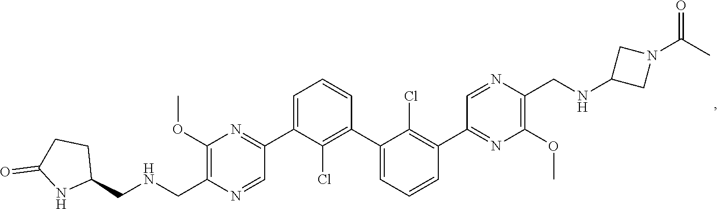 [SMILES: COc1nc(-c2cccc(-c3cccc(-c4cnc(CNC5CN(C(C)=O)C5)c(OC)n4)c3Cl)c2Cl)cnc1CNC[C@@H]1CCC(=O)N1]

or a pharmaceutically acceptable salt thereof, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

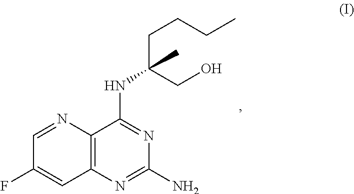

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3′-phosphate, each u is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and the compound:

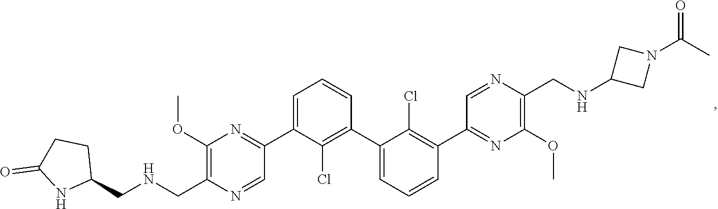 [SMILES: COc1nc(-c2cccc(-c3cccc(-c4cnc(CNC5CN(C(C)=O)C5)c(OC)n4)c3Cl)c2Cl)cnc1CNC[C@@H]1CCC(=O)N1]

or a pharmaceutically acceptable salt thereof, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

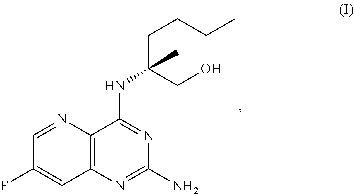

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and the compound:

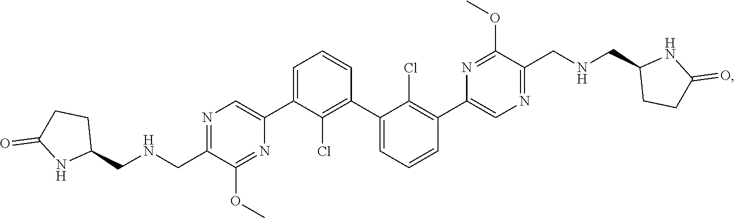 [SMILES: COc1nc(-c2cccc(-c3cccc(-c4cnc(CNC[C@@H]5CCC(=O)N5)c(OC)n4)c3Cl)c2Cl)cnc1CNC[C@@H]1CCC(=O)N1]

or a pharmaceutically acceptable salt thereof, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

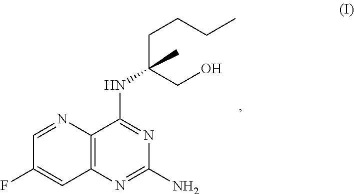

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methyl guanosine-3′-phosphate, each u is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and the compound:

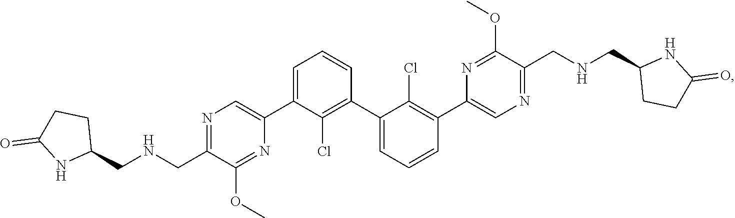 [SMILES: COc1nc(-c2cccc(-c3cccc(-c4cnc(CNC[C@@H]5CCC(=O)N5)c(OC)n4)c3Cl)c2Cl)cnc1CNC[C@@H]1CCC(=O)N1]

or a pharmaceutically acceptable salt thereof, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

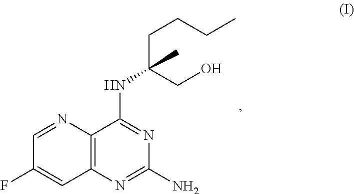

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and the compound:

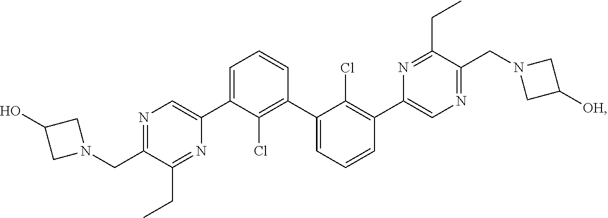 [SMILES: CCc1nc(-c2cccc(-c3cccc(-c4cnc(CN5CC(O)C5)c(CC)n4)c3Cl)c2Cl)cnc1CN1CC(O)C1]

or a pharmaceutically acceptable salt thereof, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

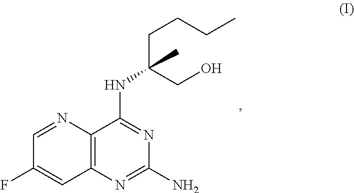

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3′-phosphate, each u is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and the compound:

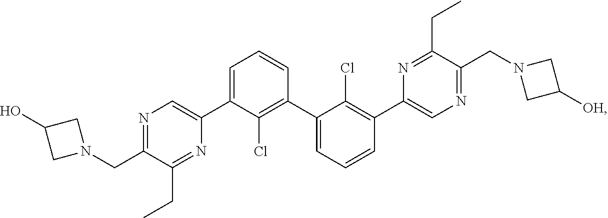 [SMILES: CCc1nc(-c2cccc(-c3cccc(-c4cnc(CN5CC(O)C5)c(CC)n4)c3Cl)c2Cl)cnc1CN1CC(O)C1]

or a pharmaceutically acceptable salt thereof, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

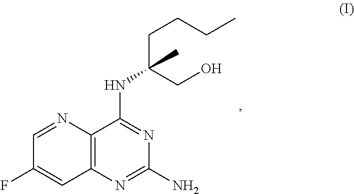

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and the compound:

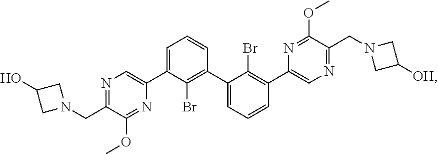 [SMILES: COc1nc(-c2cccc(-c3cccc(-c4cnc(CN5CC(O)C5)c(OC)n4)c3Br)c2Br)cnc1CN1CC(O)C1]

or a pharmaceutically acceptable salt thereof, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising a compound of Formula (I):

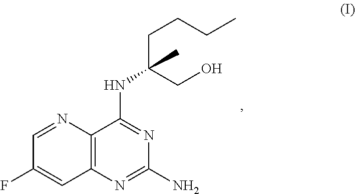

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methyl guanosine-3-phosphate, each u is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and the compound:

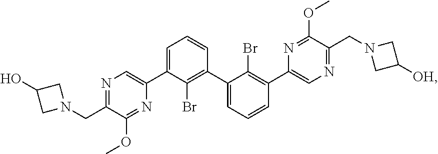 [SMILES: COc1nc(-c2cccc(-c3cccc(-c4cnc(CN5CC(O)C5)c(OC)n4)c3Br)c2Br)cnc1CN1CC(O)C1]

or a pharmaceutically acceptable salt thereof, thereby treating and/or preventing the hepatitis B viral infection in the subject.

### 1. Additional Agents

In certain embodiments, pharmaceutical compositions including an agent of the present disclosure, or a pharmaceutically acceptable salt thereof, in combination with one or more (e.g., one, two, three, four, one or two, one to three, or one to four) additional therapeutic agents, and a pharmaceutically acceptable excipient are provided.

In certain embodiments, kits including an agent of the present disclosure, or a pharmaceutically acceptable salt thereof, in combination with one or more (e.g., one, two, three, four, one or two, one to three, or one to four) additional therapeutic agents are provided.

In certain embodiments, an agent of the present disclosure, or a pharmaceutically acceptable salt thereof, is combined with one, two, three, four or more additional therapeutic agents. In certain embodiments, an agent of the present disclosure, or a pharmaceutically acceptable salt thereof, is combined with two additional therapeutic agents. In certain embodiments, an agent of the present disclosure, or a pharmaceutically acceptable salt thereof, is combined with three additional therapeutic agents. In certain embodiments, an agent of the present disclosure, or a pharmaceutically acceptable salt thereof, is combined with four additional therapeutic agents. The one, two, three, four or more additional therapeutic agents can be different therapeutic agents selected from the same class of therapeutic agents, and/or they can be selected from different classes of therapeutic agents.

In certain embodiments, when an agent of the present disclosure is combined with one or more additional therapeutic agents as described herein, the components of the composition are administered as a simultaneous or sequential regimen. When administered sequentially, the combination may be administered in two or more administrations.

Co-administration of an agent disclosed herein with one or more additional therapeutic agents generally refers to simultaneous or sequential administration of an agent disclosed herein and one or more additional therapeutic agents, such that therapeutically effective amounts of each agent are present in the body of the patient.

Co-administration includes administration of unit dosages of the agents disclosed herein before or after administration of unit dosages of one or more additional therapeutic agents. The agent disclosed herein may be administered within seconds, minutes, or hours of the administration of one or more additional therapeutic agents. For example, in some embodiments, a unit dose of an agent disclosed herein is administered first, followed within seconds or minutes by administration of a unit dose of one or more additional therapeutic agents. Alternatively, in other embodiments, a unit dose of one or more additional therapeutic agents is administered first, followed by administration of a unit dose of an agent disclosed herein within seconds or minutes. In some embodiments, a unit dose of an agent disclosed herein is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of an agent disclosed herein.

In certain embodiments, an agent of the present disclosure is combined with one or more additional therapeutic agents in a unitary dosage form for simultaneous administration to a patient, for example as a solid dosage form for oral administration.

In certain embodiments, an agent of the present disclosure, is combined with one, two, three, four or more additional therapeutic agents selected from HBV combination drugs, HBV vaccines, HBV DNA polymerase inhibitors, immunomodulators, toll-like receptor (TLR) modulators, interferon alpha receptor ligands, hyaluronidase inhibitors, hepatitis B core antigen (HBcAg) inhibitors, hepatitis B surface antigen (HBsAg) inhibitors, cytotoxic T-lymphocyte-associated protein 4 (ipi4) inhibitors, cyclophilin inhibitors, HBV viral entry inhibitors, antisense oligonucleotide targeting viral mRNA, short interfering RNAs (siRNA) and ddRNAi, endonuclease modulators, ribonucelotide reductase inhibitors, HBV E antigen inhibitors, covalently closed circular DNA (cccDNA) inhibitors, farnesoid X receptor agonists, STING agonists, anti-HBV antibodies, CCR2 chemokine antagonists, Caspase-9 stimulator, CD3 modulator, thymosin agonists, cytokines, nucleoprotein modulators, retinoic acid-inducible gene 1 stimulators, NOD2 stimulators, phosphatidylinositol 3-kinase (PI3K) inhibitors, indoleamine-2, 3-dioxygenase (IDO) pathway inhibitors, ZCCHC14 inhibitors, inducers of tertiary lymphoid aggregates, nucleic acid polymers (e.g., NAPs and STOPS), PD-1 inhibitors, PD-L1 inhibitors, recombinant thymosin alpha-1, bruton's tyrosine kinase (BTK) inhibitors, KDM inhibitors, HBV replication inhibitors, arginase inhibitors, gene therapy and cell therapy, gene editors, CAR-T cell therapy, TCR-T cell therapy, and other HBV drugs.

In certain embodiments, an agent as described herein, may be used or combined with one or more of a chemotherapeutic agent, an immunomodulator, an immunotherapeutic agent, a therapeutic antibody, a therapeutic vaccine, a bispecific antibody and “antibody-like” therapeutic protein (such as DARPins®, anti-pMHC TCR-like antibodies, DARTs®, Duobodies®, Bites®, XmAbs®, TandAbs®, Fab derivatives), an antibody-drug conjugate (ADC), gene modifiers or gene editors (such as CRISPR Cas9, zinc finger nucleases, homing endonucleases, homing meganucleases (e.g., ARCUS), synthetic nucleases, TALENs), cell therapies such as CAR-T (chimeric antigen receptor T-cell), and TCR-T (an engineered T cell receptor) agent or any combination thereof.

In certain embodiments, an agent as described herein is combined with one, two, three, four or more additional therapeutic agents, e.g., as 3-dioxygenase (IDO) inhibitors, apolipoprotein A1 modulator, arginase inhibitors, B- and T-lymphocyte attenuator inhibitors, Bruton's tyrosine kinase (BTK) inhibitors, CCR2 chemokine antagonist, CD137 inhibitors, CD160 inhibitors, CD305 inhibitors, CD4 agonist and modulator, compounds targeting hepatitis B core antigen (HBcAg), core protein allosteric modulators, covalently closed circular DNA (cccDNA) inhibitors, cyclophilin inhibitors, cytotoxic T-lymphocyte-associated protein 4 (ipi4) inhibitors, DNA polymerase inhibitor, endonuclease modulators, epigenetic modifiers, Farnesoid X receptor agonists, free fatty acid (Ffa) receptor 2 (Ffar2; PR43) agonists, free fatty acid (Ffa) receptor 3 (Ffar3; GPR441) agonists, HBV DNA polymerase inhibitors, HBV replication inhibitors, HBV RNAse inhibitors, HBV viral entry inhibitors, HBx inhibitors, Hepatitis B large envelope protein inhibitor, Hepatitis B large envelope protein stimulator, Hepatitis B structural protein modulator, hepatitis B surface antigen (HBsAg) inhibitors, hepatitis B surface antigen (HBsAg) secretion or assembly inhibitors, hepatitis B virus E antigen inhibitors, hepatitis B virus replication inhibitors, Hepatitis virus structural protein inhibitor, HIV-1 reverse transcriptase inhibitor, Hyaluronidase inhibitor, inhibitor of apoptosis proteins family proteins (IAPs) inhibitors, IL-2 agonist, IL-7 agonist, immunomodulators, indoleamine-2 inhibitors, inhibitors of ribonucleotide reductase, Interleukin-2 ligand, ipi4 inhibitors, lysine demethylase inhibitors, histone demethylase inhibitors, KDM1 inhibitors, KDM5 inhibitors, killer cell lectin-like receptor subfamily G member 1 inhibitors, lymphocyte-activation gene 3 inhibitors, lymphotoxin beta receptor activators, modulators of Axl, modulators of B7-H3, modulators of B7-H4, modulators of CD160, modulators of CD161, modulators of CD27, modulators of CD47, Non canonical RNA polymerase PAPD5 inhibitors. Non canonical RNA polymerase PAPD7 inhibitors, modulators of CD70, modulators of GITR, modulators of HEVEM, modulators of ICOS, modulators of Mer, modulators of NKG2A, modulators of NKG2D, modulators of OX40, modulators of SIRPalpha, modulators of TIGIT, modulators of Tim-4, modulators of Tyro, Na+-taurocholate cotransporting polypeptide (NTCP) inhibitors, natural killer cell receptor 2B4 inhibitors, NOD2 gene stimulator, Nucleoprotein inhibitor, nucleoprotein modulators, OX-40 receptor agonist, PD-1 inhibitors, PD-L1 inhibitors, peptidylprolyl isomerase inhibitor, phosphatidylinositol-3 kinase (PI3K) inhibitors, Retinoic acid-inducible gene 1 stimulator, Reverse transcriptase inhibitor, Ribonuclease inhibitor, RNA DNA polymerase inhibitor, SLC10A1 gene inhibitor, SMAC mimetics, Src tyrosine kinase inhibitor, stimulator of interferon gene (STING) agonists, stimulators of NOD1, T cell surface glycoprotein CD28 inhibitor, T-cell surface glycoprotein CD8 modulator, Thymosin agonist, Thymosin alpha 1 ligand, Tim-3 inhibitors, TLR-3 agonists, TLR-7 agonists, TLR-7 modulators, TLR-8 modulators, TLR-9 agonists, TLR9 agonists or gene stimulator, toll-like receptor (TLR) modulators, viral ribonucleotide reductase inhibitors, and combinations thereof.

### HBV Combination Drugs

Examples of combination drugs for the treatment of HBV include, but are not limited to, TRUVADA® (tenofovir disoproxil fumarate and emtricitabine), and adefovir.

### Other HBV Drugs

Examples of other drugs for the treatment of HBV include, but are not limited to, alpha-hydroxytropolones, amdoxovir, antroquinonol, beta-hydroxycytosine nucleosides, ARB-199, CCC-0975, ccc-R08, elvucitabine, ezetimibe, cyclosporin A, gentiopicrin (gentiopicroside), HH-003, hepalatide, JNJ-56136379, nitazoxanide, birinapant, NJK14047, NOV-205 (molixan, BAM-205), oligotide, mivotilate, feron, GST-HG-131, levamisole, Ka Shu Ning, alloferon, WS-007, Y-101 (Ti Fen Tai), rSIFN-co, PEG-IIFNm, KW-3, BP-Inter-014, oleanolic acid, HepB-nRNA, cTP-5 (rTP-5), HSK-II-2, HEISCO-106-1, HEISCO-106, Hepbarna, IBPB-0061A, Hepuyinfen, DasKloster 0014-01, ISA-204, Jiangantai (Ganxikang), MIV-210, OB-AI-004, PF-06, picroside, DasKloster-0039, hepulantai, IMB-2613, NCO-48 Fumarate, XTYW-001, SFA-001, TCM-800B, reduced glutathione, RO-6864018, ENOB-HB-01, RG-7834, QL-007sofosbuvir, ledipasvir, UB-551, PA-1010, HPN-BV1, STSG-0002, and ZH-2N, and the compounds disclosed in US20150210682, (Roche), US 2016/0122344 (Roche), WO2015173164, WO2016023877, US2015252057A (Roche), WO16128335A1 (Roche), WO16120186A1 (Roche), US2016237090A (Roche), WO16107833A1 (Roche), WO16107832A1 (Roche), US2016176899A (Roche), WO16102438A1 (Roche), WO16012470A1 (Roche), US2016220586A (Roche), and US2015031687A (Roche).

### HBV Vaccines

HBV vaccines include both prophylactic and therapeutic vaccines. Examples of HBV prophylactic vaccines include, but are not limited to, Vaxelis, Hexaxim, Heplisav, Mosquirix, DTwP-HBV vaccine, Bio-Hep-B, D/T/P/HBV/M (LBVP-0101; LBVW-0101), DTwP-Hepb-Hib-IPV vaccine, Heberpenta L, DTwP-HepB-Hib, V-419, CVI-HBV-001, Tetrabhay, hepatitis B prophylactic vaccine (Advax Super D), Hepatrol-07, GSK-223192A, ENGERIX B®, recombinant hepatitis B vaccine (intramuscular, Kangtai Biological Products), recombinant hepatitis B vaccine (Hansenual polymorpha yeast, intramuscular, Hualan Biological Engineering), recombinant hepatitis B surface antigen vaccine, Bimmugen, CARG-101, Euforavac, Eutravac, anrix-DTaP—IPV-Hep B, HBAI-20, Infanrix-DTaP—IPV-Hep B-Hib, Pentabio Vaksin DTP—HB-Hib, Comvac 4, Twinrix, Euvax-B, Tritanrix HB, Infanrix Hep B, Comvax, DTP-Hib-HBV vaccine, DTP-HBV vaccine, Yi Tai, Heberbiovac HB, Trivac HB, GerVax, DTwP-Hep B-Hib vaccine, Bilive, Hepavax-Gene, SUPERVAX, Comvac5, Shanvac-B, Hebsulin, Recombivax HB, Revac B mcf, Revac B+, Fendrix, DTwP-HepB-Hib, DNA-001, Shan5, Shan6, rhHBsAG vaccine, HBI pentavalent vaccine, LBVD, Infanrix HeXa, YS-HBV-001, IR-101H, TVAX-008, and DTaP-rHB-Hib vaccine.

Examples of HBV therapeutic vaccines include, but are not limited to, HBsAG-HBIG complex, ARB-1598, Bio-Hep-B, NASVAC, abi-HB (intravenous), ABX-203, Tetrabhay, GX-110E, GS-4774, peptide vaccine (epsilonPA-44), Hepatrol-07, NASVAC (NASTERAP), IMP-321, BEVAC, Revac B mcf, Revac B+, MGN-1333, KW-2, CVI-HBV-002, AltraHepB, VGX-6200, FP-02, FP-02.2 (HepTcell), NU-500, HBVax, im/TriGrid/antigen vaccine, Mega-CD40L-adjuvanted vaccine, HepB-v, RG7944 (INO-1800), recombinant VLP-based therapeutic vaccine (HBV infection, VLP Biotech), hepatitis B therapeutic DNA vaccine, AdTG-17909, AdTG-17910 AdTG-18202, ChronVac-B, TG-1050, VVX-001, GSK-3528869A (ChAdl55-hli-HBV+MVA-HBV+Hbc-HBs/AS01B-4), VBI-2601, VTP-300 (ChAdOx1-SIi-HBV-CPmut-TPA-Ssh prime and MVA-SIi-HBV-CPmut-TPA-Ssh boost), MVA-BN, AVA-2100, HBV-ADV311, YS-HBV-002, and Lm HBV. HBV Arenavirus vaccines are described, e.g., in WO2017076988 and WO2017198726.

### HBV DNA Polymerase Inhibitors

Examples of HBV DNA polymerase inhibitors include, but are not limited to, adefovir (HEPSERA®), emtricitabine (EMTRIVA®), tenofovir disoproxil fumarate (VIREAD®), tenofovir alafenamide, tenofovir, tenofovir disoproxil, tenofovir alafenamide fumarate, tenofovir alafenamide hemifumarate, tenofovir dipivoxil, tenofovir dipivoxil fumarate, tenofovir octadecyloxyethyl ester, CMX-157, tenofovir exalidex, besifovir, entecavir (BARACLUDE®), entecavir maleate, telbivudine (TYZEKA®), filocilovir, pradefovir, clevudine, ribavirin, lamivudine (EPIVIR-HBV®), phosphazide, famciclovir, fusolin, metacavir, ATI-2173, SNC-019754, FMCA, AGX-1009, AR-II-04-26, HIP-1302, tenofovir disoproxil aspartate, tenofovir disoproxil orotate, AiB-001, and HS-10234.

### Immunomodulators

Examples of immunomodulators include, but are not limited to, rintatolimod, imidol hydrochloride, ingaron, dermaVir, plaquenil (hydroxychloroquine), proleukin, hydroxyurea, mycophenolate mofetil (MPA) and its ester derivative mycophenolate mofetil (MMF), JNJ-440, WF-10, AB-452, ribavirin, IL-12, INO-9112, polymer polyethyleneimine (PEI), Gepon, VGV-1, MOR-22, CRV-431, JNJ-0535, TG-1050, ABI-H2158, BMS-936559, GS-9688, RO-7011785, RG-7854, RO-6871765, AIC-649, and IR-103.

### Toll-Like Receptor (TLR) Modulators

In various embodiments, the agents as described herein are combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793), TLR11, TLR12 and TLR13.

Examples of TLR modulators include, but are not limited to, AK-0701

Examples of TLR3 modulators include, but are not limited to, rintatolimod, poly-ICLC, RIBOXXON®, Apoxxim, RIBOXXIM®, IPH-33, MCT-465, MCT-475 and ND-1.1.

Examples of TLR4 agonists include, but are not limited to, G-100, and GSK-1795091.

Example TLR7 agonists that can be co-administered include without limitation AL-034, DSP-0509, GS-9620 (vesatolimod), LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics).

An TLR7/TLR8 agonist that can be co-administered is NKTR-262, telratolimod and BDB-001.

Examples of TLR-8 inhibitors include, but are not limited to, ZG-170607

Example TLR8 agonists that can be co-administered include without limitation E-6887, IMO-4200, IMO-8400, IMO-9200, MCT-465, MEDI-9197, motolimod, resiquimod, selgantolimod (GS-9688), HRS-9950, VTX-1463, VTX-763, 3M-051, 3M-052, and the compounds disclosed in US2016289229 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), US20130251673 (Novira Therapeutics), U.S. Pat. No. 9,670,205 (Gilead Sciences, Inc.), US20160289229 (Gilead Sciences, Inc.), WO2017/048727 (Gilead Sciences, Inc.), US20180065938 (Gilead Sciences, Inc.), and US20180086755 (Gilead Sciences, Inc.).

Example TLR9 agonists that can be co-administered include without limitation AST-008, cobitolimod, CMP-001, IMO-2055, IMO-2125, S-540956, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042.

Examples of TLR7, TLR8 and TLR9 modulators include, but are not limited to, the compounds disclosed in WO2017047769 (Teika Seiyaku), WO2015014815 (Janssen), WO2018045150 (Gilead Sciences Inc), WO2018045144 (Gilead Sciences Inc), WO2015162075 (Roche), WO2017034986 (University of Kansas), WO2018095426 (Jiangsu Hengrui Medicine Co Ltd), WO2016091698 (Roche), WO2016075661 (GlaxoSmithKline Biologicals), WO2016180743 (Roche), WO2018089695 (Dynavax Technologies), WO2016055553 (Roche), WO2015168279 (Novartis), WO2016107536 (Medshine Discovery), WO2018086593 (Livo (Shanghai) Pharmaceutical), WO2017106607 (Merck), WO2017061532 (Sumitomo Dainippon Pharma), WO2016023511 (Chia Tai Tianqing Pharmaceutical), WO2017076346 (Chia Tai Tianqing Pharmaceutical), WO2017046112 (Roche), WO2018078149 (Roche), WO2017040233 (3M Co), WO2016141092 (Gilead Sciences), WO2018049089 (BristolMyers Squibb), WO2015057655 (Eisai Co Ltd), WO2017001307 (Roche), WO2018005586 (BristolMyers Squibb), WO201704023 (3M Co), WO2017163264 (Council of Scientific and Industrial Research (India)), WO2018046460 (GlaxoSmithKline Biologicals), WO2018047081 (Novartis), WO2016142250 (Roche), WO2015168269 (Novartis), WO201804163 (Roche), WO2018038877 (3M Co), WO2015057659 (Eisai Co Ltd), WO2017202704 (Roche), WO2018026620 (BristolMyers Squibb), WO2016029077 (Janus Biotherapeutics), WO201803143 (Merck), WO2016096778 (Roche), WO2017190669 (Shanghai De Novo Pharmatech), U.S. Ser. No. 09/884,866 (University of Minnesota), WO2017219931 (Sichuan KelunBiotech Biopharmaceutical), WO2018002319 (Janssen Sciences), WO2017216054 (Roche), WO2017202703 (Roche), WO2017184735 (IFM Therapeutics), WO2017184746 (IFM Therapeutics), WO2015088045 (Takeda Pharmaceutical), WO2017038909 (Takeda Pharmaceutical), WO2015095780 (University of Kansas), and WO2015023958 (University of Kansas).

In certain embodiments, an agent as described herein is co-administered with a TLR7, TLR8 or TLR9 agonist.

### Interferon Alpha Receptor Ligands

Examples of interferon alpha receptor ligands include interferon alpha-2b (INTRON A©), pegylated interferon alpha-2a (PEGASYS®), PEGylated interferon alpha-1b, interferon alpha 1b (HAPGEN®), Veldona, Infradure, Roferon-A, YPEG-interferon alfa-2a (YPEG-rhIFNalpha-2a), P-1101, Algeron, Alfarona, Ingaron (interferon gamma), rSIFN-co (recombinant super compound interferon), Ypeginterferon alfa-2b (YPEG-rhIFNalpha-2b), MOR-22, peginterferon alfa-2b (PEG-INTRON®), Bioferon, Novaferon, Inmutag (Inferon), MULTIFERON®, interferon alfa-n1 (HUMOFERON®), interferon beta-1a (AVONEX®), Shaferon, interferon alfa-2b (Axxo), Alfaferone, interferon alfa-2b (BioGeneric Pharma), interferon-alpha 2 (CJ), Laferonum, VIPEG, BLAUFERON-A, BLAUFERON-B, Intermax Alpha, Realdiron, Lanstion, Pegaferon, PDferon-B, interferon alfa-2b (IFN, Laboratorios Bioprofarma), alfainterferona 2b, Kalferon, Pegnano, Feronsure, PegiHep, interferon alfa 2b (Zydus-Cadila), interferon alfa 2a, Optipeg A, Realfa 2B, Reliferon, interferon alfa-2b (Amega), interferon alfa-2b (Virchow), ropeginterferon alfa-2b, rHSA-IFN alpha-2a (recombinant human serum albumin interferon alpha 2a fusion protein), PEG-IFN-alpha, rHSA-IFN alpha 2b, recombinant human interferon alpha-(1b, 2a, 2b), peginterferon alfa-2b (Amega), peginterferon alfa-2a, Reaferon-EC, Proquiferon, Uniferon, Urifron, interferon alfa-2b (Changchun Institute of Biological Products), Anterferon, Shanferon, Layfferon, Shang Sheng Lei Tai, INTEFEN, SINOGEN, Fukangtai, Pegstat, rHSA-IFN alpha-2b, SFR-9216, and Interapo (Interapa).

### Hyaluronidase Inhibitors

Examples of hyaluronidase inhibitors include, but are not limited to, astodrimer.

### Hepatitis B Surface Antigen (HBsAg) Inhibitors

Examples of HBsAg inhibitors include, but are not limited to, AK-074, HBF-0259, GP-605, PBHBV-001, PBHBV-2-15, PBHBV-2-1, REP-9AC, REP-9C, REP-9, REP-2139, REP-2139-Ca, REP-2055, REP-2163, REP-2165, REP-2053, REP-2031, REP-006, and REP-9AC′.

Examples of HBsAg secretion inhibitors include, but are not limited to, BM601, GST-HG-131, AB-452, and ALG-010093.

### Cytotoxic T-Lymphocyte-Associated Protein 4 (CTLA4) Inhibitors

Examples of Cytotoxic T-lymphocyte-associated protein 4 (CTLA4) inhibitors include, but are not limited to, AGEN-2041, AGEN-1884, ipilumimab, belatacept, PSI-001, PRS-010, Probody mAbs, tremelimumab, and JHL-1155.

### Cyclophilin Inhibitors

Examples of cyclophilin inhibitors include, but are not limited to, CPI-431-32, EDP-494, OCB-030, SCY-635, NVP-015, NVP-018, NVP-019, STG-175, and the compounds disclosed in U.S. Pat. No. 8,513,184 (Gilead Sciences), US20140030221 (Gilead Sciences), US20130344030 (Gilead Sciences), and US20130344029 (Gilead Sciences).

### HBV Viral Entry Inhibitors

Examples of HBV viral entry inhibitors include, but are not limited to, Myrcludex B.

### Hepatitis B Large Envelope Protein Inhibitors

Examples of Hepatitis B large envelope protein inhibitors include, but are not limited to, GP-605, GST-HG-121, ALG-010093, and ALG-01013.

Antisense Oligonucleotide Targeting Viral mRNA

Examples of antisense oligonucleotide targeting viral mRNA include, but are not limited to, ISIS-HBVRx, IONIS-HBVRx, IONIS-HBV-LRx, IONIS-GSK6-LRx, GSK-3389404, BNC-1701 and RG-6004.

Short Interfering RNAs (siRNA) and ddRNAi

Examples of siRNA include, but are not limited to, TKM-HBV (TKM-HepB), ALN-HBV, SR-008, HepB-nRNA, ARC-520, ARC-521, ARB-1740, ARB-1467, AB-729, DCR-HBVS, RG-6084 (PD-L1), RG-6217, ALN-HBV-02, JNJ-3989 (ARO-HBV), STSG-0002, ALG-010133, ALG-ASO, LUNAR-HBV and DCR-HBVS (DCR-S219).

Examples of DNA-directed RNA interference (ddRNAi) include BB-HB-331.

### Endonuclease Modulators

Examples of endonuclease modulators include, but are not limited to, PGN-514.

### Ribonucelotide Reductase Inhibitors

Examples of inhibitors of ribonucleotide reductase include, but are not limited to, Trimidox.

### Nonnucleoside Reverse Transcriptase Inhibitor

Examples of Nonnucleoside Reverse Transcriptase Inhibitors (NNRTIs) include, but are not limited to, the compounds disclosed in WO2018118826 (Merck), WO2018080903 (Merck), WO2018119013 (Merck), WO2017100108 (Idenix), WO2017027434 (Merck), WO2017007701 (Merck), WO2008005555 (Gilead).

### HBV Replication Inhibitors

Examples of hepatitis B virus replication inhibitors include, but are not limited to, GP-31502, isothiafludine, IQP-HBV, RM-5038, and Xingantie.

### HIV-1 Reverse Transcriptase Inhibitors

Examples of HIV-1 reverse transcriptase inhibitors include, but are not limited to, 2,5,6-substituted pyrimidone derivative (HBV).

### Non Canonical RNA Polymerase PAPD5 and PAPD7 Inhibitors

Examples of Non canonical RNA polymerase PAPD5 and PAPD7 inhibitors include, but are not limited to, PAPD5 and PAPD7 targeting locked nucleic acid antisense oligonucleotides (HBV infection).

Covalently Closed Circular DNA (cccDNA) Inhibitors

Examples of cccDNA inhibitors include, but are not limited to, BSBI-25, ccc-R08, and CHR-101.

### Farnesoid X Receptor Agonists

Examples of farnesoid x receptor agonists include, but are not limited to, e.g., EYP-001, cilofexor (GS-9674), EDP-305, MET-409, Tropifexor, AKN-083, RDX-023, BWD-100, LMB-763, INV-3, NTX-023-1, EP-024297 and GS-8670.

### Caspase-9 Stimulators

Examples of Caspase-9 stimulators include, but are not limited to, ENOB-HB-01.

### CD3 Modulators

Examples of CD3 modulators include, but are not limited to, IMC-1109V.

### Ffar2 and Ffar3 Agonists

Examples of Ffar2 and Ffar3 agonists include, but are not limited to, SFA-001.

### Additional HBV Antibodies

Examples of HBV antibodies targeting the surface antigens of the hepatitis B virus include, but are not limited to, lenvervimab (GC-1102), XTL-17, XTL-19, KN-003, IV Hepabulin SN, VIR-3434, and fully human monoclonal antibody therapy (hepatitis B virus infection, Humabs BioMed).

Examples of HBV antibodies, including monoclonal antibodies and polyclonal antibodies, include, but are not limited to, Zutectra, Shang Sheng Gan Di, Uman Big (Hepatitis B Hyperimmune), Omri-Hep-B, Nabi-HB, Hepatect CP, HepaGam B, igantibe, Niuliva, CT-P24, hepatitis B immunoglobulin (intravenous, pH4, HBV infection, Shanghai RAAS Blood Products), and Fovepta (BT-088).

Examples of fully human monoclonal antibodies include, but are not limited to, HBC-34.

Antibodies against HBV viral peptide/major histocompatibility complex (MHC) class I (pMHC) complexes are described, e.g., in Sastry et al., J Virol. 2011 March; 85(5):1935-42 and in WO2011062562.

### CCR2 Chemokine Antagonists

Examples of CCR2 chemokine antagonists include, but are not limited to, propagermanium.

### Thymosin Agonists

Examples of thymosin agonists include, but are not limited to, Thymalfasin, and recombinant thymosin alpha 1 (GeneScience).

### Cytokines

Examples of cytokines include, but are not limited to, recombinant IL-7, CYT-107, interleukin-2 (IL-2, Immunex), recombinant human interleukin-2 (Shenzhen Neptunus), IL-15, IL-21, IL-24, and celmoleukin.

### Interleukin Agonists

In certain embodiments, the agents described herein are combined with an interleukin agonist, such as IL-2, IL-7, IL-15, IL-10, IL-12 agonists; examples of IL-2 agonists such as proleukin (aldesleukin, IL-2); pegylated IL-2 (eg NKTR-214); modified variants of IL-2 (eg THOR-707), bempegaldesleukin, AIC-284, ALKS-4230, CUI-101, Neo-2/15; examples of IL-15 agonists, such as ALT-803, NKTR-255, and hetIL-15, interleukin-15/Fc fusion protein, AM-0015, NIZ-985, SO-C101, IL-15 Synthorin (pegylated I1-15), P-22339, and a IL-15-PD-1 fusion protein N-809; examples of IL-7 include CYT-107.

### Nucleoprotein Modulators

Nucleoprotein modulators may be either HBV core or capsid protein inhibitors. Examples of nucleoprotein modulators include, but are not limited to, GS-4882, AB-423, AB-836, AT-130, ALG-001075, ALG-001024, ALG-000184, EDP-514, GLS4, NVR-1221, NVR-3778, AL-3778, BAY 41-4109, morphothiadine mesilate, ARB-168786, ARB-880, ARB-1820, GST-HG-141, JNJ-379, JNJ-632, RG-7907, GST-HG-141, HEC-72702, KL-060332, AB-506, ABI-H0731, ABI-H3733, JNJ-440, AK-0605, HRS-5091, VNRX-9945, ABI-H2158, CB-HBV-001, AK-0605, SOC-10, SOC-11 and DVR-23.

Examples of capsid inhibitors include, but are not limited to, the compounds disclosed in US2018161307 (Gilead Sciences), US20140275167 (Novira Therapeutics), US20130251673 (Novira Therapeutics), US20140343032 (Roche), WO2014037480 (Roche), US20130267517 (Roche), WO2014131847 (Janssen), WO2014033176 (Janssen), WO2014033170 (Janssen), WO2014033167 (Janssen), WO2015/059212 (Janssen), WO2015118057 (Janssen), WO2015011281 (Janssen), WO2014184365 (Janssen), WO2014184350 (Janssen), WO2014161888 (Janssen), WO2013096744 (Novira), US20150225355 (Novira), US20140178337 (Novira), US20150315159 (Novira), US20150197533 (Novira), US20150274652 (Novira), US20150259324, (Novira), US20150132258 (Novira), U.S. Pat. No. 9,181,288 (Novira), WO2014184350 (Janssen), WO2013144129 (Roche), WO2017198744 (Roche), US 20170334882 (Novira), US 20170334898 (Roche), WO2017202798 (Roche), WO2017214395 (Enanta), WO2018001944 (Roche), WO2018001952 (Roche), WO2018005881 (Novira), WO2018005883 (Novira), WO2018011100 (Roche), WO2018011160 (Roche), WO2018011162 (Roche), WO2018011163 (Roche), WO2018036941 (Roche), WO2018043747 (Kyoto Univ), US20180065929 (Janssen), WO2016168619 (Indiana University), WO2016195982 (The Penn State Foundation), WO2017001655 (Janssen), WO2017048950 (Assembly Biosciences), WO2017048954 (Assembly Biosciences), WO2017048962 (Assembly Biosciences), US20170121328 (Novira), US20170121329 (Novira).

Examples of transcript inhibitors include, but are not limited to, the compounds disclosed in WO2017013046 (Roche), WO2017016960 (Roche), WO2017017042 (Roche), WO2017017043 (Roche), WO2017061466 (Toyoma chemicals), WO2016177655 (Roche), WO2016161268 (Enanta), WO2017001853 (Redex Pharma), WO2017211791 (Roche), WO2017216685 (Novartis), WO2017216686 (Novartis), WO2018019297 (Ginkgo Pharma), WO2018022282 (Newave Pharma), US20180030053 (Novartis), and WO2018045911 (Zhejiang Pharma).

### STING Agonists, RIG-I and NOD2 Modulators

In some embodiments, the agents described herein are combined with a stimulator of interferon genes (STING). In some embodiments, the STING receptor agonist or activator is selected from the group consisting of ADU-S100 (MIW-815), SB-11285, MK-1454, SR-8291, AdVCA0848, STINGVAX, GSK-532, SYN-STING, MSA-1, SR-8291, 5,6-dimethylxanthenone-4-acetic acid (DMXAA), cyclic-GAMP (cGAMP) and cyclic-di-AMP. In some embodiments, the agents described herein are combined with a RIG-I modulator such as RGT-100, or NOD2 modulator, such as SB-9200, and IR-103.

Examples of STING agonists include, but are not limited to, the compounds disclosed in WO 2018065360 (Biolog Life Science Institute Forschungslabor und Biochemica-Vertrieb GmbH, Germany), WO 2018009466 (Aduro Biotech), WO 2017186711 (InvivoGen), WO 2017161349 (Immune Sensor), WO 2017106740 (Aduro Biotech), US 20170158724 (Glaxo Smithkiline), WO 2017075477 (Aduro Biotech), US 20170044206 (Merck), WO 2014179760 (University of California), WO2018098203 (Janssen), WO2018118665 (Merck), WO2018118664 (Merck), WO2018100558 (Takeda), WO2018067423 (Merck), and WO2018060323 (Boehringer).

### Retinoic Acid-Inducible Gene 1 Stimulators

Examples of stimulators of retinoic acid-inducible gene 1 include, but are not limited to, inarigivir soproxil (SB-9200), SB-40, SB-44, ORI-7246, ORI-9350, ORI-7537, ORI-9020, ORI-9198, ORI-7170, and RGT-100.

### NOD2 Stimulators

Examples of stimulators of NOD2 include, but are not limited to, inarigivir soproxil (SB-9200).

### Phosphatidylinositol 3-Kinase (PI3K) Inhibitors

Examples of PI3K inhibitors include, but are not limited to, idelalisib, ACP-319, AZD-8186, AZD-8835, buparlisib, CDZ-173, CLR-457, pictilisib, neratinib, rigosertib, rigosertib sodium, EN-3342, TGR-1202, alpelisib, duvelisib, IPI-549, UCB-5857, taselisib, XL-765, gedatolisib, ME-401, VS-5584, copanlisib, CAI orotate, perifosine, RG-7666, GSK-2636771, DS-7423, panulisib, GSK-2269557, GSK-2126458, CUDC-907, PQR-309, INCB-40093, pilaralisib, BAY-1082439, puquitinib mesylate, SAR-245409, AMG-319, RP-6530, ZSTK-474, MLN-1117, SF-1126, RV-1729, sonolisib, LY-3023414, SAR-260301, TAK-117, HMPL-689, tenalisib, voxtalisib, and CLR-1401.

### Immune Checkpoint Modulators

In various embodiments, the agents as described herein, are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu et al., J Exp Clin Cancer Res. (2018) 37:110). In various embodiments, the immune checkpoint proteins or receptors regulate NK cell responses (e.g., reviewed in Davis et al., Semin Immunol. (2017) 31:64-75 and Chiossone et al., Nat Rev Immunol. (2018) 18(11):671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAETIE; ULBP4); retinoic acid early transcript 1G (RAETIG; ULBP5); retinoic acid early transcript 1L (RAETIL; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CDI59A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); and SLAM family member 7 (SLAMF7).

In various embodiments, the agents described herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCDILG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSFi4 (HVEML); CD272 (B and T lymphocyte associated (BTLA)); PVR related immunoglobulin domain containing (PVRIG, CDI12R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the agents, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu et al., J Exp Clin Cancer Res. (2018) 37:110.

In various embodiments, the agents as described herein, are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CDI59A); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis et al., Semin Immunol. (2017) 31:64-75; Fang et al., Semin Immunol. (2017) 31:37-54; and Chiossone et al., Nat Rev Immunol. (2018) 18(11):671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of GS-4224, GS-4416, INCB086550 and MAX10181. Additional examples of small molecule PD-L1 inhibitors include, but are not limited to, those disclosed in U.S. Publication No. US2018305315 (Gilead Sciences), US2020017471 (Gilead Sciences) and US2019270727 (Gilead Sciences). In some embodiments, the small molecule inhibitor of CTLA4 comprises BPI-002.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDI0680 (AMP-514), spartalizumab, atezolizumab, avelumab, durvalumab, ALN-PDL, BMS-936559, CK-301, PF-06801591, BGB-108, BGB-A317 (tislelizumab), GLS-010 (WBP-3055), AK-103 (HX-008), GB-226, AK-105, CS-1003, HLX-10, MGA-012, BI-754091, PDR-001, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, RO-6084 (PD-L1 antisense oligonucleotide), STI-1110, GX-P2, RG-7446, mDX-400, (MSB0010718C), CX-072, CBT-502, TSR-042 (dostarlimab), MSB-2311, JTX-4014, BGB-A333, SHR-1316, CS-1001 (WBP-3155), MEDI-0680, envafolimab (KN-035), KD-033, KY-1003, IBI-308 (sintilimab), HLX-20, KL-A167, STI-A1014, STI-A1015 (IMC-001), BCD-135, FAZ-053, TQB-2450, MDX1105-01, MSB-0010718C, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFO-EC domain), CA-170 (PD-L1/VISTA), CDX-527 (CD27/PD-L1), LY-3415244 (TIM3/PDL1), GNS-1480 (Epidermal growth factor receptor antagonist; Programmed cell death ligand 1 inhibitor), M-7824 (PD-L1/TGF-βbifunctional fusion protein), and INBRX-105 (4-1BB/PDL1).

Examples of PD-Iinhibitors include, but are not limited to, the compounds disclosed in WO2017112730 (Incyte Corp), WO2017087777 (Incyte Corp), WO2017017624, WO2014151634 (BristolMyers Squibb Co), WO201317322 (BristolMyers Squibb Co), WO2018119286 (Incyte Corp), WO2018119266 (Incyte Corp), WO2018119263 (Incyte Corp), WO2018119236 (Incyte Corp), WO2018119221 (Incyte Corp), WO2018118848 (BristolMyers Squibb Co), WO20161266460 (BristolMyers Squibb Co), WO2017087678 (BristolMyers Squibb Co), WO2016149351 (BristolMyers Squibb Co), WO2015033299 (Aurigene Discovery Technologies Ltd), WO2015179615 (Eisai Co Ltd; Eisai Research Institute), WO2017066227 (BristolMyers Squibb Co), WO2016142886 (Aurigene Discovery Technologies Ltd), WO2016142852 (Aurigene Discovery Technologies Ltd), WO2016142835 (Aurigene Discovery Technologies Ltd; Individual), WO2016142833 (Aurigene Discovery Technologies Ltd), WO2018085750 (BristolMyers Squibb Co), WO2015033303 (Aurigene Discovery Technologies Ltd), WO2017205464 (Incyte Corp), WO2016019232 (3M Co; Individual; Texas A&M University System), WO2015160641 (BristolMyers Squibb Co), WO2017079669 (Incyte Corp), WO2015033301 (Aurigene Discovery Technologies Ltd), WO2015034820 (BristolMyers Squibb Co), WO2018073754 (Aurigene Discovery Technologies Ltd), WO2016077518 (BristolMyers Squibb Co), WO2016057624 (BristolMyers Squibb Co), WO2018044783 (Incyte Corp), WO2016100608 (BristolMyers Squibb Co), WO2016100285 (BristolMyers Squibb Co), WO2016039749 (BristolMyers Squibb Co), WO2015019284 (Cambridge Enterprise Ltd), WO2016142894 (Aurigene Discovery Technologies Ltd), WO2015134605 (BristolMyers Squibb Co), WO2018051255 (Aurigene Discovery Technologies Ltd), WO2018051254 (Aurigene Discovery Technologies Ltd), WO2017222976 (Incyte Corp), WO2017070089 (Incyte Corp), WO2018044963 (BristolMyers Squibb Co), WO2013144704 (Aurigene Discovery Technologies Ltd), WO2018013789 (Incyte Corp), WO2017176608 (BristolMyers Squibb Co), WO2018009505 (BristolMyers Squibb Co), WO2011161699 (Aurigene Discovery Technologies Ltd), WO2015119944 (Incyte Corp; Merck Sharp & Dohme Corp), WO2017192961 (Incyte Corp), WO2017106634 (Incyte Corp), WO2013132317 (Aurigene Discovery Technologies Ltd), WO2012168944 (Aurigene Discovery Technologies Ltd), WO2015036927 (Aurigene Discovery Technologies Ltd), WO2015044900 (Aurigene Discovery Technologies Ltd), and WO2018026971 (Arising International).

In various embodiments, the agents as described herein are combined with anti-TIGIT antibodies, such as BMS-986207, RG-6058, and AGEN-1307.

### TNF Receptor Superfamily (TNFRSF) Member Agonists or Activators

In various embodiments, the agents as described herein are combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Example anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, IBI-101 and those described in WO2016179517, WO2017096179, WO2017096182, WO2017096281, and WO2018089628.

Example anti-TNFRSFS (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Example anti-TNFRSF9 (4-1BB, CD137) antibodies that can be co-administered include without limitation urelumab, utomilumab (PF-05082566), AGEN2373 and ADG-106.

Example anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

### Indoleamine-pyrrole-2,3-dioxygenase (IDO1) Inhibitors

In various embodiments, the agents as described herein, are combined with an inhibitor of indoleamine 2,3-dioxygenase 1 (IDO1; NCBI Gene ID: 3620). Examples of IDO1 inhibitors include without limitation, BLV-0801, epacadostat, resminostat, F-001287, GBV-1012, GBV-1028, GDC-0919, indoximod, NKTR-218, NLG-919-based vaccine, PF-06840003, pyranonaphthoquinone derivatives (SN-35837), SBLK-200802, BMS-986205, and shIDO-ST, EOS-200271, KHK-2455, LY-3381916, and the compounds disclosed in US20100015178 (Incyte), US2016137652 (Flexus Biosciences, Inc.), WO2014073738 (Flexus Biosciences, Inc.), and WO2015188085 (Flexus Biosciences, Inc.).

### LAG-3 and TIM-3 Inhibitors

In certain embodiments, the agents as described herein are combined with an anti-TIM-3 antibody, such as TSR-022, LY-3321367, MBG-453, and INCAGN-2390.

In certain embodiments, the agents described herein are combined with an anti LAG-3 (Lymphocyte-activation) antibody, such as relatlimab (ONO-4482), LAG-525, MK-4280, REGN-3767, and INCAGN2385.

Examples of additional immune-based therapies that can be combined with an agent of this disclosure include interferon alfa; interferon alfa-2b; interferon alfa-n3; pegylated interferon alfa; interferon gamma; Flt3 agonists; gepon; normferon, peginterferon alfa-2a, peginterferon alfa-2b, RPI-MN.

### Inhibitor of Apoptosis Proteins Family Proteins (IAPs)

Examples of IAP inhibitors include, but are not limited to, APG-1387.

### Recombinant Thymosin Alpha-1

Examples of recombinant thymosin alpha-1 include, but are not limited to, NL-004 and PEGylated thymosin alpha-1.

### Bruton's Tyrosine Kinase (BTK) Inhibitors

Examples of BTK inhibitors include, but are not limited to, ABBV-105, acalabrutinib (ACP-196), ARQ-531, BMS-986142, dasatinib, ibrutinib, GDC-0853, PRN-1008, SNS-062, ONO-4059, BGB-3111, ML-319, MSC-2364447, RDX-022, X-022, AC-058, RG-7845, spebrutinib, TAS-5315, TP-0158, TP-4207, HM-71224, KBP-7536, M-2951, TAK-020, AC-0025, and the compounds disclosed in US20140330015 (Ono Pharmaceutical), US20130079327 (Ono Pharmaceutical), and US20130217880 (Ono Pharmaceutical).

### KDM Inhibitors

Examples of KDM5 inhibitors include, but are not limited to, the compounds disclosed in WO2016057924 (Genentech/Constellation Pharmaceuticals), US20140275092 (Genentech/Constellation Pharmaceuticals), US20140371195 (Epitherapeutics), US20140371214 (Epitherapeutics), US20160102096 (Epitherapeutics), US20140194469 (Quanticel), US20140171432, US20140213591 (Quanticel), US20160039808 (Quanticel), US20140275084 (Quanticel), and WO2014164708 (Quanticel).

Examples of KDM1 inhibitors include, but are not limited to, the compounds disclosed in U.S. Pat. No. 9,186,337B2 (Oryzon Genomics), GSK-2879552, RG-6016, and ORY-2001.

### Arginase Inhibitors

Examples of Arginase inhibitors include, but are not limited to, e CB-1158, C-201, and resminostat.

### Bi-and Tri-Specific Natural Killer (NK)-Cell Engagers

In various embodiments, the agents as described herein, are combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). As appropriate, the anti-CD16 binding bi-specific molecules may or may not have an Fc. Illustrative bi-specific NK-cell engagers that can be co-administered target CD16 and one or more HBV-associated antigens as described herein. BiKEs and TriKEs are described, e.g., in Felices, et al., Methods Mol Biol. (2016) 1441:333-346; Fang, et al., Semin Immunol. (2017) 31:37-54.

### Long Acting Treatments

Long acting entecavir (subcutaneous depot), long acting tenofovir (TFD and TAF) implants (devices) or subcutaneous depot. An example of long acting entecavir is described in Exploration of long-acting implant formulations of hepatitis B drug entecavir., Eur J Pharm Sci. 2019 Aug. 1; 136:104958.

### Gene Therapy and Cell Therapy

In certain embodiments, the agents described herein are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection.

### Gene Editors

The genome editing system is selected from the group consisting of: a CRISPR/Cas9 system, a zinc finger nuclease system, a TALEN system, a homing endonucleases system, and a meganuclease system (e.g., an ARCUS system); e.g., cccDNA elimination via targeted cleavage, and altering one or more of the hepatitis B virus (HBV) viral genes. Altering (e.g., knocking out and/or knocking down) the PreC, C, X, PreS1, PreS2, S, P or SP gene refers to (1) reducing or eliminating PreC, C, X, PreS1, PreS2, S, P or SP gene expression, (2) interfering with Precore, Core, X protein, Long surface protein, middle surface protein, S protein (also known as HBs antigen and HBsAg), polymerase protein, and/or Hepatitis B spliced protein function (HBe, HBc, HBx, PreS1, PreS2, S, Pol, and/or HBSP or (3) reducing or eliminating the intracellular, serum and/or intraparenchymal levels of HBe, HBc, HBx, LHBs, MHBs, SHBs, Pol, and/or HBSP proteins. Knockdown of one or more of the PreC, C, X, PreS1, PreS2, S, P and/or SP gene(s) is performed by targeting the gene(s) within HBV cccDNA and/or integrated HBV DNA. Additional examples genome editing systems include, but are not limited to, those disclosed in US2019284543 (Gilead Sciences), and US2019338263 (Gilead Sciences).

Example of gene therapy, such as liver targeted anti-HBV gene therapy (using ARCUS technology), or using CRISPR/Cas9 gene editing technology, or EBT-106 (LNP-delivered CRISPR/CasX nuclease.

### CAR-T Cell Therapy

CAR-T cell therapy includes, but is not limited to, a population of immune effector cells engineered to express a chimeric antigen receptor (CAR), wherein the CAR includes an HBV antigen-binding domain. In certain embodiments, the antigen-binding domain is a domain disclosed herein. In certain embodiments, the antigen-binding domain is other than a domain disclosed herein. In certain embodiments, the antigen is HBsAg (i.e., HbsAg-CART). The immune effector cell is a T-cell or an NK cell. In certain embodiments, the T-cell is a CD4+ T-cell, a CD8+ T-cell, a NK cell or a combination thereof. Cells can be autologous or allogeneic. An example of a CART directed to HBV is described in Cytotherapy. 2018 May; 20(5):697-705. doi: 10.1016/j.jcyt.2018.02.

### TCR-T Cell Therapy

TCR-T cell therapy includes, but is not limited to, T cells expressing HBV-specific T cell receptors. TCR-T cells are engineered to target HBV derived peptides presented on the surface of virus-infected cells. An example of a TCR directed to HBV is described in Wisskirchen, K. et al. T cell receptor grafting allows virological control of hepatitis B virus infection. J Clin Invest. 2019; 129(7):2932-2945.

TCR-T cell therapy includes, but is not limited to, T-Cells expressing HBV surface antigen (HBsAg)-specific TCR.

TCR-T cell therapy includes, but is not limited to, TCR-T therapy directed to treatment of HBV, such as LTCR-H2-1.

In another specific embodiment, an agent disclosed herein, or a pharmaceutically acceptable salt thereof, is combined with an HBV DNA polymerase inhibitor, one or two additional therapeutic agents selected from the group consisting of immunomodulators, TLR modulators, HBsAg inhibitors, HBsAg secretion or assembly inhibitors, HBV therapeutic vaccines, HBV antibodies including HBV antibodies targeting the surface antigens of the hepatitis B virus and bispecific antibodies and “antibody-like” therapeutic proteins (such as DARTs®, DUOBODIES®, BITES®, XmAbs®, TandAbs®, Fab derivatives, or TCR-like antibodies), cyclophilin inhibitors, stimulators of retinoic acid-inducible gene 1, stimulators of RIG-I like receptors, PD-1 inhibitors, PD-L1 inhibitors, Arginase inhibitors, PI3K inhibitors, IDO inhibitors, and stimulators of NOD2, and one or two additional therapeutic agents selected from the group consisting of HBV viral entry inhibitors, NTCP inhibitors, HBx inhibitors, cccDNA inhibitors, HBV antibodies targeting the surface antigens of the hepatitis B virus, siRNA, miRNA gene therapy agents, sshRNAs, KDM5 inhibitors, and nucleoprotein modulators (HBV core or capsid protein modulators).

In another specific embodiment, an agent disclosed herein, or a pharmaceutically acceptable salt thereof, is combined with at least a second additional therapeutic agent selected from the group consisting of: HBV DNA polymerase inhibitors, immunomodulator, TLR modulators, HBsAg inhibitors, HBV therapeutic vaccines, HBV antibodies including HBV antibodies targeting the surface antigens of the hepatitis B virus and bispecific antibodies and “antibody-like” therapeutic proteins (such as DARPins®, anti-pMHC TCR-like antibodies, DARTs®, DUOBODIES®, BITES®, XmAbs®, TandAbs®, Fab derivatives, or TCR-like antibodies), cyclophilin inhibitors, stimulators of retinoic acid-inducible gene 1, stimulators of RIG-I like receptors, PD-1 inhibitors, PD-L1 inhibitors, Arginase inhibitors, PI3K inhibitors, IDO inhibitors, and stimulators of NOD2.

In another specific embodiment, an agent disclosed herein, or a pharmaceutically acceptable salt thereof, is combined with at least a second additional therapeutic agent selected from the group consisting of: HBV DNA polymerase inhibitors, HBV viral entry inhibitors, NTCP inhibitors, HBx inhibitors, cccDNA inhibitors, HBV antibodies targeting the surface antigens of the hepatitis B virus, siRNA, miRNA gene therapy agents, sshRNAs, KDM5 inhibitors, and nucleoprotein modulators (HBV core or capsid protein inhibitors).

In a particular embodiment, an agent disclosed herein, or a pharmaceutically acceptable salt thereof, is combined with compounds such as those disclosed in U.S.

Publication No. 2010/0143301 (Gilead Sciences), U.S. Publication No. 2011/0098248 (Gilead Sciences), U.S. Publication No. 2009/0047249 (Gilead Sciences), U.S. Pat. No. 8,722,054 (Gilead Sciences), U.S. Publication No. 2014/0045849 (Janssen), U.S. Publication No. 2014/0073642 (Janssen), WO2014/056953 (Janssen), WO2014/076221 (Janssen), WO2014/128189 (Janssen), U.S. Publication No. 2014/0350031 (Janssen), WO2014/023813 (Janssen), U.S. Publication No. 2008/0234251 (Array Biopharma), U.S. Publication No. 2008/0306050 (Array Biopharma), U.S. Publication No. 2010/0029585 (Ventirx Pharma), U.S. Publication No. 2011/0092485 (Ventirx Pharma), US2011/0118235 (Ventirx Pharma), U.S. Publication No. 2012/0082658 (Ventirx Pharma), U.S. Publication No. 2012/0219615 (Ventirx Pharma), U.S. Publication No. 2014/0066432 (Ventirx Pharma), U.S. Publication No. 2014/0088085 (Ventirx Pharma), U.S. Publication No. 2014/0275167 (Novira Therapeutics), U.S. Publication No. 2013/0251673 (Novira Therapeutics), U.S. Pat. No. 8,513,184 (Gilead Sciences), U.S. Publication No. 2014/0030221 (Gilead Sciences), U.S. Publication No. 2013/0344030 (Gilead Sciences), U.S. Publication No. 2013/0344029 (Gilead Sciences), US20140275167 (Novira Therapeutics), US20130251673 (Novira Therapeutics), U.S. Publication No. 2014/0343032 (Roche), WO2014037480 (Roche), U.S. Publication No. 2013/0267517 (Roche), WO2014131847 (Janssen), WO2014033176 (Janssen), WO2014033170 (Janssen), WO2014033167 (Janssen), WO2015/059212 (Janssen), WO2015118057 (Janssen), WO2015011281 (Janssen), WO2014184365 (Janssen), WO2014184350 (Janssen), WO2014161888 (Janssen), WO2013096744 (Novira), US20150225355 (Novira), US20140178337 (Novira), US20150315159 (Novira), US20150197533 (Novira), US20150274652 (Novira), US20150259324, (Novira), US20150132258 (Novira), U.S. Pat. No. 9,181,288 (Novira), WO2014184350 (Janssen), WO2013144129 (Roche), US20100015178 (Incyte), US2016137652 (Flexus Biosciences, Inc.), WO2014073738 (Flexus Biosciences, Inc.), WO2015188085 (Flexus Biosciences, Inc.), U.S. Publication No. 2014/0330015 (Ono Pharmaceutical), U.S. Publication No. 2013/0079327 (Ono Pharmaceutical), U.S. Publication No. 2013/0217880 (Ono pharmaceutical), WO2016057924 (Genentech/Constellation Pharmaceuticals), US20140275092 (Genentech/Constellation Pharmaceuticals), US20140371195 (Epitherapeutics) and US20140371214 (Epitherapeutics)., US20160102096 (Epitherapeutics), US20140194469 (Quanticel), US20140171432, US20140213591 (Quanticel), US20160039808 (Quanticel), US20140275084 (Quanticel), WO2014164708 (Quanticel), U.S. Pat. No. 9,186,337B2 (Oryzon Genomics), and other drugs for treating HBV, and combinations thereof.

In certain embodiments, an agent disclosed herein, or a pharmaceutically acceptable salt thereof, is combined with 5-30 mg tenofovir alafenamide fumarate, tenofovir alafenamide hemifumarate, or tenofovir alafenamide. In certain embodiments, an agent disclosed herein, or a pharmaceutically acceptable salt thereof, is combined with 5-10; 5-15; 5-20; 5-25; 25-30; 20-30; 15-30; or 10-30 mg tenofovir alafenamide fumarate, tenofovir alafenamide hemifumarate, or tenofovir alafenamide. In certain embodiments, an agent disclosed herein, or a pharmaceutically acceptable salt thereof, is combined with 10 mg tenofovir alafenamide fumarate, tenofovir alafenamide hemifumarate, or tenofovir alafenamide. In certain embodiments, an agent disclosed herein, or a pharmaceutically acceptable salt thereof, is combined with 25 mg tenofovir alafenamide fumarate, tenofovir alafenamide hemifumarate, or tenofovir alafenamide. An agent as disclosed herein may be combined with the agents provided herein in any dosage amount of the compound (e.g., from 50 mg to 500 mg of compound) the same as if each combination of dosages were specifically and individually listed.

In certain embodiments, an agent disclosed herein, or a pharmaceutically acceptable salt thereof, is combined with 100-400 mg tenofovir disoproxil fumarate, tenofovir disoproxil hemifumarate, or tenofovir disoproxil. In certain embodiments, an agent disclosed herein, or a pharmaceutically acceptable salt thereof, is combined with 100-150; 100-200, 100-250; 100-300; 100-350; 150-200; 150-250; 150-300; 150-350; 150-400; 200-250; 200-300; 200-350; 200-400; 250-350; 250-400; 350-400 or 300-400 mg tenofovir disoproxil fumarate, tenofovir disoproxil hemifumarate, or tenofovir disoproxil. In certain embodiments, an agent disclosed herein, or a pharmaceutically acceptable salt thereof, is combined with 300 mg tenofovir disoproxil fumarate, tenofovir disoproxil hemifumarate, or tenofovir disoproxil. In certain embodiments, an agent disclosed herein, or a pharmaceutically acceptable salt thereof, is combined with 250 mg tenofovir disoproxil fumarate, tenofovir disoproxil hemifumarate, or tenofovir disoproxil. In certain embodiments, an agent herein, or a pharmaceutically acceptable salt thereof, is combined with 150 mg tenofovir disoproxil fumarate, tenofovir disoproxil hemifumarate, or tenofovir disoproxil. An agent as disclosed herein may be combined with the agents provided herein in any dosage amount of the compound (e.g., from 50 mg to 500 mg of compound) the same as if each combination of dosages were specifically and individually listed.

### B. Pharmaceutically Acceptable Salts

Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, di-substituted cycloalkyl amine, tri-substituted cycloalkyl amines, cycloalkenyl amines, di(cycloalkenyl) amines, tri(cycloalkenyl) amines, substituted cycloalkenyl amines, di-substituted cycloalkenyl amine, tri-substituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Amines are of general structure N(R³⁰)(R³¹)(R³²) wherein mono-substituted amines have two of the three substituents on nitrogen (R³⁰, R³¹ and R³²) as hydrogen, di-substituted amines have one of the three substituents on nitrogen (R³⁰, R³¹, and R³²) as hydrogen, whereas tri-substituted amines have none of the three substituents on nitrogen (R³⁰, R³¹, and R³²) as hydrogen. R³⁰, R³¹, and R³² are selected from a variety of substituents such as hydrogen, optionally substituted alkyl, aryl, heteroaryl, cycloalkyl, cycloalkenyl, heterocyclyl, and the like.

Specific examples of suitable amines include, by way of example only, isopropyl amine, trimethyl amine, diethyl amine, tri(iso-propyl) amine, tri(n-propyl) amine, ethanolamine, diethanolamine, 2-dimethylamino ethanol, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

### C. Administration

The combination therapy regimens of the present disclosure can be delivered by any suitable means, including oral, parenteral and topical methods. Other administration methods include intravenous administration and subcutaneous administration.

“Intravenous administration” is the administration of substances directly into a vein, or “intravenously.” Compared with other routes of administration, the intravenous (IV) route is a faster way to deliver fluids and medications throughout the body. An infusion pump can allow precise control over the flow rate and total amount of medication delivered. However, in cases where a change in the flow rate would not have serious consequences, or if pumps are not available, the drip is often left to flow simply by placing the bag above the level of the patient and using the clamp to regulate the rate. Alternatively, a rapid infuser can be used if the patient requires a high flow rate and the IV access device is of a large enough diameter to accommodate it. This is either an inflatable cuff placed around the fluid bag to force the fluid into the patient or a similar electrical device that may also heat the fluid being infused. When a patient requires medications only at certain times, intermittent infusion is used which does not require additional fluid. It can use the same techniques as an intravenous drip (pump or gravity drip), but after the complete dose of medication has been given, the tubing is disconnected from the IV access device. Some medications are also given by IV push or bolus, meaning that a syringe is connected to the IV access device and the medication is injected directly (slowly, if it might irritate the vein or cause a too-rapid effect). Once a medicine has been injected into the fluid stream of the IV tubing there must be some means of ensuring that it gets from the tubing to the patient. Usually this is accomplished by allowing the fluid stream to flow normally and thereby carry the medicine into the bloodstream; however, a second fluid injection is sometimes used, as a “flush”, following the injection to push the medicine into the bloodstream more quickly. Thus in one embodiment, compound(s) or combination of compounds described herein may be administered by IV administration alone or in combination with administration of certain components of the treatment regimen by oral or parenteral routes.

“Oral administration” is a route of administration where a substance is taken through the mouth, and includes buccal, sub labial, and sublingual administration, as well as enteral administration and that through the respiratory tract, unless made through e.g., tubing so the medication is not in direct contact with any of the oral mucosa. Typical form for the oral administration of therapeutic agents includes the use of tablets or capsules. Thus in one embodiment, compound(s) or combination of compounds described herein may be administered by oral route alone or in combination with administration of certain components of the treatment regimen by IV or parenteral routes.

The components of the combination therapy regimen of the present disclosure can be administered at any suitable frequency, interval and duration. For example, each component of the combination therapy regimen of the present disclosure can be administered once an hour, or two, three or more times an hour, once a day, or two, three, or more times per day, or once every 2, 3, 4, 5, 6, or 7 days, so as to provide the preferred dosage level.

Each component of the combination therapy regimen of the present disclosure can be administered once a week, or once every 2, 3, 4, 5, 6, 7, 8, 9, 10, 11, 12, 13, 14, 15, 16, 17, 18, 19, 20, 21, 22, 23, 24, 25 or 26 weeks, so as to provide the preferred dosage level. When the components of the combination therapy regimen of the present disclosure are administered more than once a day, representative intervals include 5, 10, 15, 20, 30, 45 and 60 minutes, as well as 1, 2, 4, 6, 8, 10, 12, 16, 20, and 24 hours. Each component of the combination therapy regimen of the present disclosure can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The combination therapy regimen can also include other compatible therapeutic agents. The components described herein can be used in combination with one another, with other active agents, or with adjunctive agents that may not be effective alone, but may contribute to the efficacy of the active agent.

In some embodiments, the compound of Formula (I) is administered at any suitable time as known by one of skill in the art. Representative time periods for administration of the compound of Formula (I) include, but are not limited to, about 4 weeks, or 8, 12, 16, 20, 24, 28, 32, 36, 40, 44, 48, 52, 56, 60, 64, 68, 72, 76, 80, 84, 88, 92, 96, 100, or about 104 weeks. In some embodiments, the compound of Formula (I) is administered once a week for 12 weeks to 60 weeks. In some embodiments, the compound of Formula (I) is administered once a week for 12 weeks to 48 weeks. In some embodiments, the compound of Formula (I) is administered once a week for 12 weeks to 24 weeks.

In some embodiments, the compound of Formula (I) is administered once a week for 104 weeks. In some embodiments, the compound of Formula (I) is administered once a week for 52 weeks. In some embodiments, the compound of Formula (I) is administered once a week for 48 weeks. In some embodiments, the compound of Formula (I) is administered once a week for 24 weeks.

The compound of Formula (I) can be administered to the subject by any suitable means including, but not limited to, oral administration. In some embodiments, the compound of Formula (I) is administered orally. In some embodiments, the compound of Formula (I) is administered orally once a week for 24 weeks.

In some embodiments, the dsRNA is administered at any suitable time as known by one of skill in the art. For example, the dsRNA can be administered once a week, or once every 2, 3, 4, 5, 6, 7, 8, 9, 10, 11, 12, 13, 14, 15, 16, 17, 18, 19, 20, 21, 22, 23, 24, 25 or 26 weeks. Representative time periods for administration of the dsRNA include, but are not limited to, about 4 weeks, or 8, 12, 16, 20, 24, 28, 32, 36, 40, 44, 48, 52, 56, 60, 64, 68, 72, 76, 80, 84, 88, 92, 96, 100, or about 104 weeks. In some embodiments, the dsRNA is administered once every week, once every 2 weeks, once every 4 weeks, once every 6 weeks, once every 8 weeks, or once every 12 weeks for any suitable time as known by one of skill in the art. In some embodiments, the dsRNA is administered for 12 weeks to 60 weeks. In some embodiments, the dsRNA is administered for 12 weeks to 48 weeks. In some embodiments, the dsRNA is administered for 12 weeks to 24 weeks.

In some embodiments, the dsRNA is administered once every 4 weeks for 104 weeks. In some embodiments, the dsRNA is administered once every 12 weeks for 104 weeks. In some embodiments, the dsRNA is administered once every 4 weeks for 52 weeks. In some embodiments, the dsRNA is administered once every 12 weeks for 52 weeks. In some embodiments, the dsRNA is administered once every 4 weeks for 48 weeks. In some embodiments, the dsRNA is administered once every 12 weeks for 48 weeks. In some embodiments, the dsRNA is administered once every 12 weeks for 24 weeks. In some embodiments, the dsRNA is administered once every 4 weeks for 24 weeks.

The dsRNA can be administered to the subject by any suitable means including, but not limited to, intravenous injection or subcutaneous injection. In some embodiments, the dsRNA is administered by subcutaneous injection. In some embodiments, the dsRNA is administered by subcutaneous injection once every 4 weeks for 24 weeks. In some embodiments, the dsRNA is administered by intravenous injection. In some embodiments, the dsRNA is administered by intravenous injection once every 4 weeks for 24 weeks.

In some embodiments, the PD-1/PD-L1 inhibitors described herein can be administered at any suitable time as known by one of skill in the art. For example, the PD-1/PD-L1 inhibitor can be administered once a week, or once every 2, 3, 4, 5, 6, 7, 8, 9, 10, 11, 12, 13, 14, 15, 16, 17, 18, 19, 20, 21, 22, 23, 24, 25 or 26 weeks. In some embodiments, the PD-1/PD-L1 inhibitors described herein can be administered once every week, once every 2 weeks, once every 4 weeks, once every 6 weeks, once every 8 weeks, or once every 12 weeks for any suitable time as known by one of skill in the art. Representative time periods for administration of the PD-1/PD-L1 inhibitor include, but are not limited to, about 4 weeks, or 8, 12, 16, 20, 24, 28, 32, 36, 40, 44, 48, 52, 56, 60, 64, 68, 72, 76, 80, 84, 88, 92, 96, 100, or about 104 weeks. In some embodiments, the PD-1/PD-L1 inhibitors is administered for 12 weeks to 60 weeks. In some embodiments, the PD-1/PD-L1 inhibitors described herein is administered for 12 weeks to 48 weeks. In some embodiments, the PD-1/PD-L1 inhibitors is administered for 12 weeks to 24 weeks.

In some embodiments, the PD-1/PD-L1 inhibitor is nivolumab. In some embodiments, the nivolumab can be administered at any suitable time as known by one of skill in the art. For example, the nivolumab can be administered once a week, or once every 2, 3, 4, 5, 6, 7, 8, 9, 10, 11, 12, 13, 14, 15, 16, 17, 18, 19, 20, 21, 22, 23, 24, 25 or 26 weeks. In some embodiments, the nivolumab can be administered once every week, once every 2 weeks, once every 4 weeks, once every 6 weeks, once every 8 weeks, or once every 12 weeks for any suitable time as known by one of skill in the art. Representative time periods for administration of the nivolumab include, but are not limited to, about 4 weeks, or 8, 12, 16, 20, 24, 28, 32, 36, 40, 44, 48, 52, 56, 60, 64, 68, 72, 76, 80, 84, 88, 92, 96, 100, or about 104 weeks. In some embodiments, the nivolumab is administered for 12 weeks to 60 weeks. In some embodiments, the nivolumab described herein is administered for 12 weeks to 48 weeks. In some embodiments, the nivolumab is administered for 12 weeks to 24 weeks.

In some embodiments, the nivolumab is administered once every 4 weeks for 104 weeks. In some embodiments, the nivolumab is administered once every 12 weeks for 104 weeks. In some embodiments, the nivolumab is administered once every 4 weeks for 52 weeks. In some embodiments, the nivolumab is administered once every 12 weeks for 52 weeks. In some embodiments, the nivolumab is administered once every 4 weeks for 48 weeks. In some embodiments, the nivolumab is administered once every 12 weeks for 48 weeks. In some embodiments, the nivolumab is administered once every 12 weeks for 24 weeks. In some embodiments, the nivolumab is administered once every 4 weeks for 24 weeks.

The PD-1/PD-L1 inhibitor can be administered to the subject by any suitable means including, but not limited to, orally, intravenous injection or subcutaneous injection. In some embodiments, the PD-1/PD-L1 inhibitor is administered orally. In some embodiments, the PD-1/PD-L1 inhibitor is administered by intravenous injection. In some embodiments, the PD-1/PD-L1 inhibitor is administered by subcutaneous injection. In some embodiments, the nivolumab is administered by intravenous injection once every 4 weeks for 24 weeks. In some embodiments, the nivolumab and is administered by subcutaneous injection once every 4 weeks for 24 weeks.

In some embodiments, the method comprises administering the compound of Formula I, the dsRNA, and nivolumab. In some embodiments, the method comprises administering the compound of Formula I, the dsRNA, and nivolumab at any suitable time as described herein.

In some embodiments, the dsRNA is administered by subcutaneous injection once every 4 weeks for 36 weeks starting at day 1. In some embodiments, the dsRNA is administered by subcutaneous injection once every 4 weeks for 24 weeks starting at day 1.

In some embodiments, the compound of Formula I is administered while the subject is fasting. In some embodiments, the compound of Formula I is administered orally once a week for 48 weeks starting at day 1 while the subject is fasting. In some embodiments, the compound of Formula I is administered orally once a week for 36 weeks starting at day 1 while the subject is fasting.

In some embodiments, the nivolumab is administered by intravenous injection once every 4 weeks for 36 weeks starting at day 1. In some embodiments, the nivolumab is administered by intravenous injection once every 4 weeks for 24 weeks starting at day 1.

In some embodiments, the compound of Formula I is administered orally once a week for 24 weeks starting at day 1 while the subject is fasting, the dsRNA is administered by subcutaneous injection once every 4 weeks for 24 weeks starting at day 1, and the nivolumab is administered by intravenous injection once every 4 weeks for 24 weeks starting at day 1.

In some embodiments, the compound of Formula I is administered orally once a week for 24 weeks starting at week 12 while the subject is fasting. In some embodiments, the nivolumab is administered by subcutaneous injection or intravenous injection every 4 weeks for 24 weeks starting at week 12. In some embodiments, the nivolumab is administered by intravenous injection every 4 weeks for 24 weeks starting at week 12.

In some embodiments, the compound of Formula I is administered orally once a week for 24 weeks starting at week 12 while the subject is fasting, the dsRNA is administered by subcutaneous injection once every 4 weeks for 24 weeks starting at day 1, and the nivolumab is administered by intravenous injection every 4 weeks for 24 weeks starting at week 12.

The combination therapy regimen of the present disclosure can exclude a nucleotide. In some embodiments, the subject is not administered a nucleotide. In some embodiments, provided herein is a method of treating and/or preventing a hepatitis B viral infection in a subject in need thereof, comprising administering to the subject a therapeutically effective amount of a combination therapy regimen consisting of a compound of Formula (I):

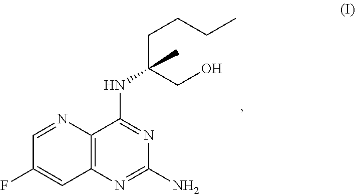

or a pharmaceutically acceptable salt thereof, a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and a PD-1/PD-L1 inhibitor, or a pharmaceutically acceptable salt thereof, thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen consisting of a compound of Formula (I):

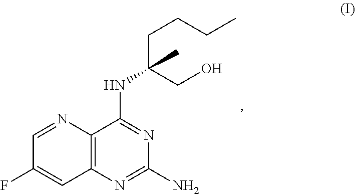

or a pharmaceutically acceptable salt thereof, a double stranded ribonucleic acid (dsRNA) comprising SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methyl guanosine-3-phosphate, each u is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and a PD-1/PD-L1 inhibitor, or a pharmaceutically acceptable salt thereof, thereby treating and/or preventing the hepatitis B viral infection in the subject.

In some embodiments, the combination therapy regimen includes at least one additional agent. In some embodiments, the additional agent includes tenofovir alafenamide, or tenofovir alafenamide fumarate. In some embodiments, the additional agent is tenofovir alafenamide. In some embodiments, the additional agent is tenofovir alafenamide fumarate.

In some embodiments, the method of the present disclosure further comprises administering to the subject an additional therapeutic agent.

In some embodiments, the method further comprises administering to the subject a compound of Formula (II):

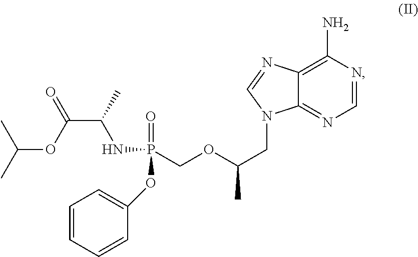 [SMILES: CC(C)OC(=O)[C@H](C)N[P@](=O)(CO[C@H](C)Cn1cnc2c(N)ncnc21)Oc1ccccc1]

or a pharmaceutically acceptable salt thereof.

In some embodiments, the compound of Formula II has the structure:

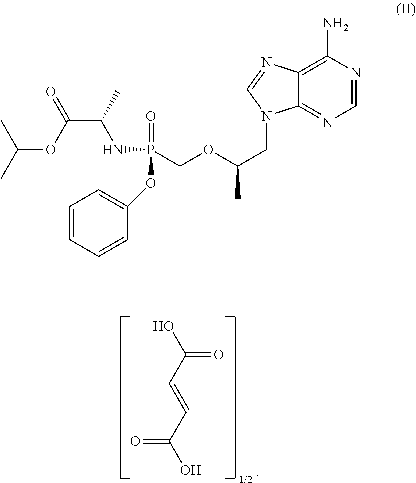

In some embodiments, the compound of Formula II has the structure:

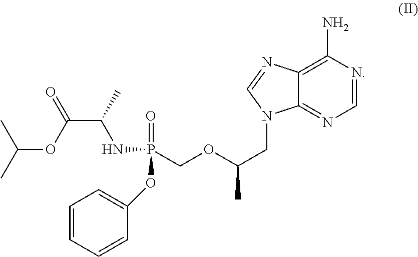 [SMILES: CC(C)OC(=O)[C@H](C)N[P@](=O)(CO[C@H](C)Cn1cnc2c(N)ncnc21)Oc1ccccc1]

The compound of Formula II can be administered by any suitable method and within any suitable time as described herein. The compound of Formula II can be administered for any suitable period of time including, but not limited to, 4 weeks, 8, 12, 16, 20, 24, 28, 32, 36, 40, 44, 48, 52, 56, 60, 64, 68, 72, 76, 80, 84, 88, 92, 96, 100 or about 104 weeks. In some embodiments, the compound of Formula II is administered orally. In some embodiments, the compound of Formula II is administered once daily for 84 weeks starting at day 1. In some embodiments, the compound of Formula II is administered once daily for 48 weeks starting at day 1. In some embodiments, the compound of Formula II is administered once daily for 42 weeks starting at day 1. In some embodiments, the compound of Formula II is administered once daily for 104 weeks starting at day 1. In some embodiments, the compound of Formula II is administered once daily for 36 to 84 weeks starting at day 1. In some embodiments, the compound of Formula II is administered once daily for 52 weeks starting at day 1. In some embodiments, the compound of Formula II is administered once daily for at least 36 weeks starting at day 1. In some embodiments, the compound of Formula II is administered once daily for 36 weeks starting at day 1. In some embodiments, the compound of Formula II is administered once daily for 24 weeks starting at day 1.

In some embodiments, administration of the compound of Formula II is terminated if after 36 weeks the subject is characterized by: (i) a hepatitis B viral load of less than about 20 international units per milliliter (IU/mL); (ii) negative for the hepatitis B e-antigen (HBeAg); and (iii) a hepatitis B surface antigen (HBsAg) concentration of less than about 100 international units per milliliter (IU/mL). In some embodiments, administration of the compound of Formula II is terminated if after 36 weeks the subject is characterized by: (i) a hepatitis B viral load of less than about 20 international units per milliliter (IU/mL); (ii) negative for the hepatitis B e-antigen (HBeAg); and (iii) negative for the hepatitis B surface antigen (HBsAg).

In some embodiments, administration of the compound of Formula II is terminated if after 36 weeks the subject is characterized by: (i) a hepatitis B viral load of less than the lower limit of quantitation; (ii) negative for the hepatitis B e-antigen (HBeAg); (iii) alanine aminotransferase less than about twice the upper limit of normal; and (iv) a hepatitis B surface antigen (HBsAg) concentration of less than about 100 international units per milliliter (IU/mL). In some embodiments, administration of the compound of Formula II is terminated if after 36 weeks the subject is characterized by: (i) a hepatitis B viral load of less than the lower limit of quantitation; (ii) negative for the hepatitis B e-antigen (HBeAg); (iii) alanine aminotransferase less than about twice the upper limit of normal; and (iv) negative for the hepatitis B surface antigen (HBsAg).

In some embodiments, the method comprises administering the compound of Formula II, the compound of Formula I, the dsRNA, and nivolumab. In some embodiments, the method comprises administering the compound of Formula II, the compound of Formula I, the dsRNA, and nivolumab at any suitable time as described herein.

In some embodiments, the compound of Formula II is administered orally once daily for 48 weeks. In some embodiments, the compound of Formula II is administered orally once daily for 36 weeks. In some embodiments, the compound of Formula II is administered orally once daily for 36 weeks starting at day 1. In some embodiments, the compound of Formula II is administered orally once daily for 24 weeks. In some embodiments, the compound of Formula II is administered orally once daily for 24 weeks starting at day 1.

In some embodiments, the dsRNA is administered by subcutaneous injection once every 4 weeks for 36 weeks. In some embodiments, the dsRNA is administered by subcutaneous injection once every 4 weeks for 24 weeks. In some embodiments, the dsRNA is administered by subcutaneous injection once every 4 weeks for 24 weeks starting at day 1.

In some embodiments, the compound of Formula I is administered orally once a week for 36 weeks while the subject is fasting. In some embodiments, the compound of Formula I is administered orally once a week for 24 weeks while the subject is fasting. In some embodiments, the compound of Formula I is administered orally once a week for 24 weeks starting at week 12 while the subject is fasting.

In some embodiments, the nivolumab is administered by intravenous injection every 4 weeks for 36 weeks. In some embodiments, the nivolumab is administered by intravenous injection every 4 weeks for 24 weeks starting at day 1. In some embodiments, the nivolumab is administered by intravenous injection every 4 weeks for 24 weeks starting at week 12.

In some embodiments, the compound of Formula II is administered orally once daily for 36 weeks starting at day 1, the dsRNA is administered by subcutaneous injection once every 4 weeks for 24 weeks starting at day 1, the compound of Formula I is administered orally once a week for 24 weeks starting at week 12 while the subject is fasting, and the nivolumab is administered by intravenous injection every 4 weeks for 24 weeks starting at week 12.

### D. Formulations

Each component of the combination therapy regimen of the present disclosure or a pharmaceutically acceptable salt thereof may be administered in a pharmaceutical formulation. Pharmaceutical formulations/compositions of the present disclosure include a combination of the compound of formula (I), or a pharmaceutically acceptable salt thereof, a double stranded ribonucleic acid (dsRNA), and a PD-1-PD-L1 inhibitor, or a pharmaceutically acceptable salt thereof, optionally in combination with an additional agent such as, for example, tenofovir alafenamide, or a pharmaceutically acceptable salt thereof.

Each component of the combination therapy regimen can be administered by injection and include aqueous solutions, oil suspensions, emulsions (with sesame oil, corn oil, cottonseed oil, or peanut oil) as well as elixirs, mannitol, dextrose, or a sterile aqueous solution, and similar pharmaceutical vehicles. Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and/or by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the component compound(s) in the required amount in the appropriate solvent with various other ingredients as enumerated above or as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient(s) plus any additional desired ingredient from a previously sterile-filtered solution thereof.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

Certain compositions are preferably formulated in a unit dosage form. The term “unit dosage forms” or “combined dosage unit” refers to physically discrete units suitable as unitary dosages for human subjects and other mammals, each unit containing a predetermined quantity of one or more of the active materials (e.g., compound (I), optionally in combination with an additional agent calculated to produce the desired effect, in association with a suitable pharmaceutical excipient in for example, a tablet, capsule, ampoule or vial for injection. It will be understood, however, that the amount of each active agent actually administered will be determined by a physician, in the light of the relevant circumstances, including the condition to be treated, the chosen route of administration, the actual compounds administered and their relative activity, the age, weight, and response of the individual patient, the severity of the patient's symptoms, and the like.

For preparing solid compositions such as tablets, the principal active ingredient(s) is/are mixed with a pharmaceutical excipient to form a solid pre-formulation composition containing a homogeneous mixture of a compound of the present disclosure. When referring to these pre-formulation compositions as homogeneous, it is meant that the active ingredient(s) are dispersed evenly throughout the composition so that the composition may be readily subdivided into equally effective unit dosage forms such as tablets, pills and capsules.

Each component of the combination therapy regimen can be provided in a pharmaceutical preparation preferably in unit dosage form. In such form the preparation is subdivided into unit doses containing appropriate quantities of the compounds of the present disclosure. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Each component of the combination therapy regimen of the present disclosure can be present in any suitable amount, and can depend on various factors including, but not limited to, weight and age of the subject, state of the disease, etc. Suitable dosage ranges for the compound of the present disclosure include from about 0.1 mg to about 10,000 mg, or about 1 mg to about 1000 mg, or about 10 mg to about 750 mg, or about 25 mg to about 500 mg, or about 50 mg to about 250 mg. Suitable dosages for the compound of the present disclosure include about 1 mg, 5, 10, 20, 30, 40, 50, 60, 70, 80, 90, 100, 200, 300, 400, 500, 600, 700, 800, 900 or 1000 mg.

Each component of the combination therapy regimen of the present disclosure and the active agent can be present in the compositions of the present disclosure in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w). The compound of the present disclosure and the other active agent can be present in any suitable weight ratio, such as about 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w). Other dosages and dosage ratios of the compound of the present disclosure and the active agent are suitable in the compositions and methods of the present disclosure.

The compound of Formula I is administered in any suitable amount known by one of skill in the art. In some embodiments, the compound of Formula I is administered to the subject in an amount of 0.5 to 20 mg. In some embodiments, the compound of Formula I is administered to the subject in an amount of 1 to 10 mg. Other amounts of the compound of Formula I that can be administered to the subject include, but are not limited to, about 1.0 mg, or about 1.1, 1.2, 1.3, 1.4, 1.5, 1.6, 1.7, 1.8, 1.9, 2.0, 2.5 or about 3.0 mg. In some embodiments, the compound of Formula I is administered to the subject in an amount of about 3 mg.

The compound of Formula I can be administered in two equal amounts, or two unequal amounts. In some embodiments, the compound of Formula I can be administered in two equal amounts. In some embodiments, the compound of Formula I is administered to the subject in two 1.5 mg doses.

The dsRNA is administered in any suitable amount known by one of skill in the art. In some embodiments, the dsRNA is administered to the subject in an amount of 100 to 300 mg. In some embodiments, the dsRNA is administered to the subject in an amount of 150 to 250 mg. Representative amounts of the dsRNA administered to the subject include, but are not limited to, about 100 mg, or about 105, 110, 115, 120, 125, 130, 135, 140, 145, 150, 155, 160, 165, 170, 175, 180, 185, 190, 195, 200, 210, 220, 230, 240, or about 250 mg. In some embodiments, the dsRNA is administered to the subject in an amount of about 200 mg.

The PD-1/PD-L1 inhibitors described herein can be administered in any suitable amount known by one of skill in the art. In some embodiments, the PD-1/PD-L1 inhibitors described herein is administered to the subject in an amount of 0.01 to 5 mg/kg. In some embodiments, the PD-1/PD-L1 inhibitors described herein is administered to the subject in an amount of 0.1 to 1 mg/kg. Representative amounts of the PD-1/PD-L1 inhibitors administered to the subject include, but are not limited to, about 0.1 mg/kg, or about 0.15, 0.20, 0.25, 0.30, 0.35, 0.40, 0.45, 0.50, 0.55, 0.60, 0.65, 0.70, 0.75, 0.80, 0.85, 0.90, 0.95, or about 1.0 mg/kg. In some embodiments, the PD-1/PD-L1 inhibitors described herein is administered to the subject in an amount of 0.1 to 0.5 mg/kg. In some embodiments, the PD-1/PD-L1 inhibitors described herein is administered to the subject in an amount of about 0.3 mg/kg.

The PD-1/PD-L1 inhibitors can be administered in any suitable amount known by one of skill in the art. In some embodiments, the compound of Formula I is administered to the subject in an amount 0.1 to 1000 mg. Representative amounts of the PD-1/PD-L1 inhibitor administered to the subject include, but are not limited to, from 0.1 to 500 mg, 1 to 100 mg, 1 to 50 mg, or from 10 to 50 mg. Other amounts of the PD-1/PD-L1 inhibitor administered to the subject include, but are not limited to, about 1 mg, or 2, 3, 4, 5, 6, 7, 8, 9, 10, 15, 20, 25, 30, 35, 40, 45, 50, 55, 60, 65, 70, 75, 80, 85, 90, 95, or about 100 mg.

In some embodiments, the compound of Formula I is administered to the subject in an amount of 1 to 10 mg. Other amounts of the compound of Formula I that can be administered to the subject include, but are not limited to, about 1.0 mg, or about 1.1, 1.2, 1.3, 1.4, 1.5, 1.6, 1.7, 1.8, 1.9, 2.0, 2.5 or about 3.0 mg. In some embodiments, the compound of Formula I is administered to the subject in an amount of about 3 mg.

The nivolumab can be administered in any suitable amount known by one of skill in the art. In some embodiments, the nivolumab is administered to the subject in an amount of 0.1 to 1 mg/kg. In some embodiments, the nivolumab is administered to the subject in an amount of 0.1 to 0.5 mg/kg. In some embodiments, the nivolumab is administered to the subject in an amount of about 0.3 mg/kg.

The compound of Formula II can be administered in any suitable amount known by one of skill in the art. In some embodiments, the compound of Formula II is administered to the subject in an amount of 10 to 50 mg. In some embodiments, the compound of Formula II is administered to the subject in an amount of 20 to 40 mg. In some embodiments, the compound of Formula II is administered to the subject in an amount of 20 to 30 mg. Representative amounts of the compound of Formula II include, but are not limited to, about 1 mg, or 2, 3, 4, 5, 6, 7, 8, 9, 10, 15, 20, 25, 30, 35, 40, 45, 50, 55, 60, 65, 70, 75, 80, 85, 90, 95, or about 100 mg. In some embodiments, the compound of Formula II is administered to the subject in an amount of about 25 mg. In some embodiments, the compound of Formula II is administered to the subject in an amount of about 28 mg.

In some embodiments, the method of the present disclosure can reduce the viral load in a subject following completion of treatment. In some embodiments, the subject has a hepatitis B viral load of less than about 300 international units per milliliter (IU/mL) following completion of treatment. In some embodiments, the subject has a hepatitis B viral load of less than about 200 international units per milliliter (IU/mL) following completion of treatment. In some embodiments, the subject has a hepatitis B viral load of less than about 100 international units per milliliter (IU/mL) following completion of treatment. In some embodiments, the subject has a hepatitis B viral load of less than about 50 international units per milliliter (IU/mL) following completion of treatment. In some embodiments, the subject has a hepatitis B viral load of less than about 20 international units per milliliter (IU/mL) following completion of treatment. In some embodiments, the subject has a hepatitis B viral load of less than about 5 international units per milliliter (IU/mL) following completion of treatment. In some embodiments, the subject has a hepatitis B viral load of less than the lower limit of quantitation (LLOQ) following completion of treatment.

In some embodiments, the method of the present disclosure can reduce the hepatitis B surface antigen (HBsAg) concentration in a subject following completion of treatment. In some embodiments, the subject has a hepatitis B surface antigen (HBsAg) concentration of less than about 200 international units per milliliter (IU/mL) following completion of treatment. In some embodiments, the subject has a hepatitis B surface antigen (HBsAg) concentration of less than about 100 international units per milliliter (IU/mL) following completion of treatment. In some embodiments, the subject is negative for the hepatitis B surface antigen (HBsAg) following completion of treatment.

In some embodiments, the subject is negative for the hepatitis B e-antigen (HBeAg) following completion of treatment.

In some embodiments, the method of the present disclosure can reduce the alanine aminotransferase (ALT) concentration in a subject following completion of treatment. In some embodiments, the subject has an alanine aminotransferase (ALT) concentration of less than about 2 times the upper limit of normal (ULN). The upper limit of normal can be about 100 international units per liter (IU/L), or about 95, 90, 85, 80, 75, 70, 65, 60, 55, 50, 45, 40, 35, 30, 25, 20, 15, or about 10 IU/L. In some embodiments, the alanine aminotransferase concentration upper limit of normal is about 40 IU/L.

In some embodiments, following the termination of treatment, the subject is characterized by at least one of: (i) a hepatitis B viral load of less than about 20 international units per milliliter (IU/mL); (ii) negative for the hepatitis B e-antigen (HBeAg); and (iii) a hepatitis B surface antigen (HBsAg) concentration of less than about 100 international units per milliliter (IU/mL). In some embodiments, following the termination of treatment, the subject is characterized by at least one of: (i) a hepatitis B viral load of less than about 20 international units per milliliter (IU/mL); (ii) negative for the hepatitis B e-antigen (HBeAg); and (iii) negative for the hepatitis B surface antigen (HBsAg).

In some embodiments, following the termination of treatment, the subject is characterized by at least one of: (i) a hepatitis B viral load of less than the lower limit of quantitation; (ii) negative for the hepatitis B e-antigen (HBeAg); (iii) alanine aminotransferase less than about twice the upper limit of normal; and (iv) a hepatitis B surface antigen (HBsAg) concentration of less than about 100 international units per milliliter (IU/mL). In some embodiments, following the termination of treatment, the subject is characterized by at least one of: (i) a hepatitis B viral load of less than the lower limit of quantitation; (ii) negative for the hepatitis B e-antigen (HBeAg); (iii) alanine aminotransferase less than about twice the upper limit of normal; and (iv) negative for the hepatitis B surface antigen (HBsAg).

In some embodiments, following the termination of treatment, the subject is characterized by: (i) a hepatitis B viral load of less than about 20 international units per milliliter (IU/mL); (ii) negative for the hepatitis B e-antigen (HBeAg); and (iii) a hepatitis B surface antigen (HBsAg) concentration of less than about 100 international units per milliliter (IU/mL). In some embodiments, following the termination of treatment, the subject is characterized by: (i) a hepatitis B viral load of less than about 20 international units per milliliter (IU/mL); (ii) negative for the hepatitis B e-antigen (HBeAg); and (iii) negative for the hepatitis B surface antigen (HBsAg).

In some embodiments, following the termination of treatment, the subject is characterized by: (i) a hepatitis B viral load of less than the lower limit of quantitation; (ii) negative for the hepatitis B e-antigen (HBeAg); (iii) alanine aminotransferase less than about twice the upper limit of normal; and (iv) a hepatitis B surface antigen (HBsAg) concentration of less than about 100 international units per milliliter (IU/mL). In some embodiments, following the termination of treatment, the subject is characterized by: (i) a hepatitis B viral load of less than the lower limit of quantitation; (ii) negative for the hepatitis B e-antigen (HBeAg); (iii) alanine aminotransferase less than about twice the upper limit of normal; and (iv) negative for the hepatitis B surface antigen (HBsAg).

In some embodiments, following the termination of treatment, the subject is characterized by: (i) a hepatitis B viral load of less than about 0.05 international units per milliliter (IU/mL); (ii) negative for the hepatitis B e-antigen (HBeAg); and (iii) a hepatitis B surface antigen (HBsAg) concentration of less than about 100 international units per milliliter (IU/mL). In some embodiments, following the termination of treatment, the subject is characterized by: (i) a hepatitis B viral load of less than about 0.05 international units per milliliter (IU/mL); (ii) negative for the hepatitis B e-antigen (HBeAg); and (iii) negative for the hepatitis B surface antigen (HBsAg).

In some embodiments, following the termination of treatment, the subject is characterized by: (i) a hepatitis B viral load of less than about 0.05 international units per milliliter (IU/mL); (ii) negative for the hepatitis B e-antigen (HBeAg); (iii) alanine aminotransferase less than about twice the upper limit of normal; and (iv) a hepatitis B surface antigen (HBsAg) concentration of less than about 100 international units per milliliter (IU/mL). In some embodiments, following the termination of treatment, the subject is characterized by: (i) a hepatitis B viral load of less than about 0.05 international units per milliliter (IU/mL); (ii) negative for the hepatitis B e-antigen (HBeAg); (iii) alanine aminotransferase less than about twice the upper limit of normal; and (iv) negative for the hepatitis B surface antigen (HBsAg).

In some embodiments, provided herein is a method of treating and/or preventing a hepatitis B viral infection in a subject in need thereof, comprising administering to the subject a therapeutically effective amount of a combination therapy regimen comprising
3 mg of a compound of Formula (I):

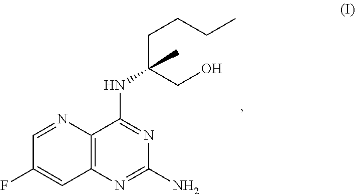

or a pharmaceutically acceptable salt thereof, wherein the compound of Formula I is administered orally once a week for 24 weeks starting at day 1 while the subject is fasting,
200 mg of a dsRNA of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein the dsRNA is administered by subcutaneous injection once every 4 weeks for 24 weeks starting at day 1, and
0.3 mg/kg nivolumab, wherein the nivolumab is administered by intravenous injection once every 4 weeks for 24 weeks starting at day 1,
wherein following completion of treatment, the subject is characterized by
   (i) a hepatitis B viral load of less than about 20 international units per milliliter (IU/mL);
   (ii) negative for the hepatitis B e-antigen (HBeAg); and
   (iii) negative for the hepatitis B surface antigen (HBsAg),
thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising
3 mg of a compound of Formula (I):

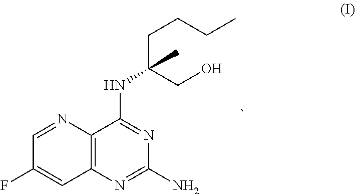

or a pharmaceutically acceptable salt thereof, wherein the compound of Formula I is administered orally once a week for 24 weeks starting at day 1 while the subject is fasting,
200 mg of a dsRNA of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein the dsRNA is administered by subcutaneous injection once every 4 weeks for 24 weeks starting at day 1, and
0.3 mg/kg nivolumab, wherein the nivolumab is administered by intravenous injection once every 4 weeks for 24 weeks starting at day 1,
wherein following completion of treatment, the subject is characterized by
   (i) a hepatitis B viral load of less than the lower limit of quantitation;
   (ii) negative for the hepatitis B e-antigen (HBeAg);
   (iii) alanine aminotransferase less than about twice the upper limit of normal; and
   (iv) negative for the hepatitis B surface antigen (HBsAg),
thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising
3 mg of a compound of Formula (I):

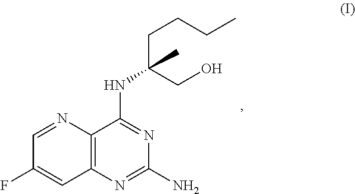

or a pharmaceutically acceptable salt thereof, wherein the compound of Formula I is administered orally once a week for 24 weeks starting at day 1 while the subject is fasting,
200 mg of a dsRNA of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3′-phosphate, each u is 2′-O-methyluridine-3′-phosphate, each Af is 2′-fluoroadenosine-3′-phosphate, each Cf is 2′-fluorocytidine-3′-phosphate, each (if is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and wherein the dsRNA is administered by subcutaneous injection once every 4 weeks for 24 weeks starting at day 1, and
0.3 mg/kg nivolumab, wherein the nivolumab is administered by intravenous injection once every 4 weeks for 24 weeks starting at day 1,
wherein following completion of treatment, the subject is characterized by
   (i) a hepatitis B viral load of less than about 20 international units per milliliter (IU/mL);
   (ii) negative for the hepatitis B e-antigen (HBeAg); and
   (iii) negative for the hepatitis B surface antigen (HBsAg),
thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising
3 mg of a compound of Formula (I):

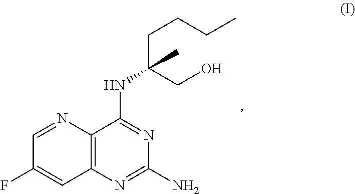

or a pharmaceutically acceptable salt thereof, wherein the compound of Formula I is administered orally once a week for 24 weeks starting at day 1 while the subject is fasting,
200 mg of a dsRNA of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2-O-methylguanosine-3′-phosphate, each u is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and wherein the dsRNA is administered by subcutaneous injection once every 4 weeks for 24 weeks starting at day 1, and
0.3 mg/kg nivolumab, wherein the nivolumab is administered by intravenous injection once every 4 weeks for 24 weeks starting at day 1,
wherein following completion of treatment, the subject is characterized by
   (i) a hepatitis B viral load of less than the lower limit of quantitation;
   (ii) negative for the hepatitis B e-antigen (HBeAg);
   (iii) alanine aminotransferase less than about twice the upper limit of normal; and
   (iv) negative for the hepatitis B surface antigen (HBsAg),
thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising
3 mg of a compound of Formula (I):

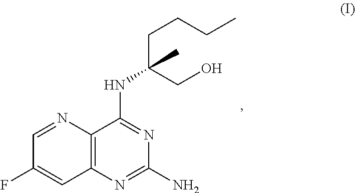

or a pharmaceutically acceptable salt thereof, wherein of the compound of Formula I is administered orally once a week for 24 weeks starting at week 12 while the subject is fasting,
200 mg of a dsRNA of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein the dsRNA is administered by subcutaneous injection once every 4 weeks for 24 weeks starting at day 1, and
0.3 mg/kg nivolumab, wherein the nivolumab is administered by intravenous injection once every 4 weeks for 24 weeks starting at week 12,
wherein following completion of treatment, the subject is characterized by
   (i) a hepatitis B viral load of less than about 20 international units per milliliter (IU/mL);
   (ii) negative for the hepatitis B e-antigen (HBeAg); and
   (iii) negative for the hepatitis B surface antigen (HBsAg),
thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising
3 mg of a compound of Formula (I):

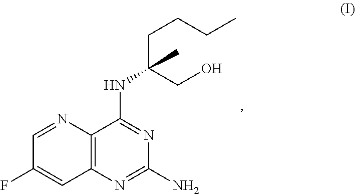

or a pharmaceutically acceptable salt thereof, wherein of the compound of Formula I is administered orally once a week for 24 weeks starting at week 12 while the subject is fasting,
200 mg of a dsRNA of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein the dsRNA is administered by subcutaneous injection once every 4 weeks for 24 weeks starting at day 1, and
0.3 mg/kg nivolumab, wherein the nivolumab is administered by intravenous injection once every 4 weeks for 24 weeks starting at week 12,
wherein following completion of treatment, the subject is characterized by
   (i) a hepatitis B viral load of less than the lower limit of quantitation;
   (ii) negative for the hepatitis B e-antigen (HBeAg);
   (iii) alanine aminotransferase less than about twice the upper limit of normal; and
   (iv) negative for the hepatitis B surface antigen (HBsAg),
thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising
3 mg of a compound of Formula (I):

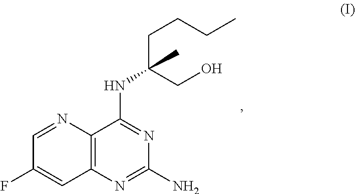

or a pharmaceutically acceptable salt thereof, wherein of the compound of Formula I is administered orally once a week for 24 weeks starting at week 12 while the subject is fasting,
200 mg of a dsRNA of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3′-phosphate, each u is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and wherein the dsRNA is administered by subcutaneous injection once every 4 weeks for 24 weeks starting at day 1, and
0.3 mg/kg nivolumab, wherein the nivolumab is administered by intravenous injection once every 4 weeks for 24 weeks starting at week 12,
wherein following completion of treatment, the subject is characterized by
   (i) a hepatitis B viral load of less than about 20 international units per milliliter (IU/mL);
   (ii) negative for the hepatitis B e-antigen (HBeAg); and
   (iii) negative for the hepatitis B surface antigen (HBsAg),
thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising
3 mg of a compound of Formula (I):

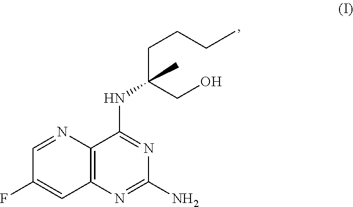

or a pharmaceutically acceptable salt thereof, wherein of the compound of Formula I is administered orally once a week for 24 weeks starting at week 12 while the subject is fasting,
200 mg of a dsRNA of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methyleytidine-3′-phosphate, each g is 2′-O-methylguanosine-3′-phosphate, each u is 2′-O-methyluridine-3′-phosphate, each Af is 2′-fluoroadenosine-3′-phosphate, each Cf is 2′-fluorocytidine-Y-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GaiNAc-alkyl)-amidodecanoyl)]-4-hydroxyprolinol, and wherein the dsRNA is administered by subcutaneous injection once every 4 weeks for 24 weeks starting at day 1, and
0.3 mg/kg nivolumab, wherein the nivolumab is administered by intravenous injection once every 4 weeks for 24 weeks starting at week 12,
wherein following completion of treatment, the subject is characterized by
   (i) a hepatitis B viral load of less than the lower limit of quantitation;
   (ii) negative for the hepatitis B e-antigen (HBeAg);
   (iii) alanine aminotransferase less than about twice the upper limit of normal; and
   (iv) negative for the hepatitis B surface antigen (HBsAg),
thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising
3 mg of a compound of Formula (I):

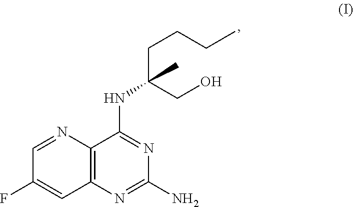

or a pharmaceutically acceptable salt thereof, wherein the compound of Formula I is administered orally once a week for 24 weeks starting at week 12 while the subject is fasting,
200 mg of a dsRNA of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein the dsRNA is administered by subcutaneous injection once every 4 weeks for 24 weeks starting at day 1,
0.3 mg/kg of nivolumab, wherein the nivolumab is administered by intravenous injection once every 4 weeks for 24 weeks starting at week 12, and
25 mg of a compound of Formula (II):

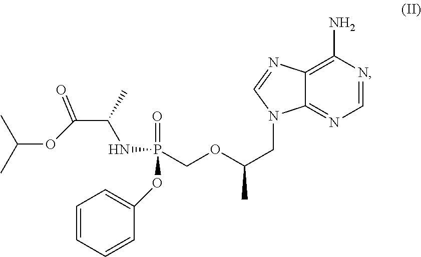 [SMILES: CC(C)OC(=O)[C@H](C)N[P@](=O)(CO[C@H](C)Cn1cnc2c(N)ncnc21)Oc1ccccc1]

wherein the compound of Formula II is administered orally once daily for 36 weeks starting at day 1,
wherein following completion of treatment, the subject is characterized by
   (i) a hepatitis B viral load of less than about 20 international units per milliliter (IU/mL);
   (ii) negative for the hepatitis B e-antigen (HBeAg); and
   (iii) negative for the hepatitis B surface antigen (HBsAg),
thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising
3 mg of a compound of Formula (I):

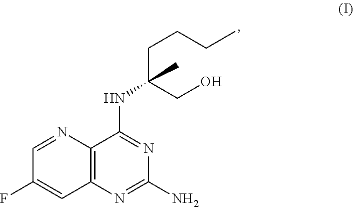

or a pharmaceutically acceptable salt thereof, wherein the compound of Formula I is administered orally once a week for 24 weeks starting at week 12 while the subject is fasting,
200 mg of a dsRNA of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein the dsRNA is administered by subcutaneous injection once every 4 weeks for 24 weeks starting at day 1,
0.3 mg/kg of nivolumab, wherein the nivolumab is administered by intravenous injection once every 4 weeks for 24 weeks starting at week 12, and
25 mg of a compound of Formula (II):

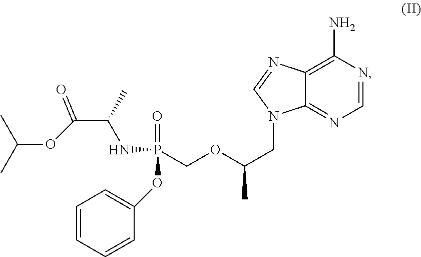 [SMILES: CC(C)OC(=O)[C@H](C)N[P@](=O)(CO[C@H](C)Cn1cnc2c(N)ncnc21)Oc1ccccc1]

wherein the compound of Formula II is administered orally once daily for 36 weeks starting at day 1,
wherein following completion of treatment, the subject is characterized by
   (i) a hepatitis B viral load of less than the lower limit of quantitation;
   (ii) negative for the hepatitis B e-antigen (HBeAg);
   (iii) alanine aminotransferase less than about twice the upper limit of normal; and
   (iv) negative for the hepatitis B surface antigen (HBsAg),
thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising
3 mg of a compound of Formula (I):

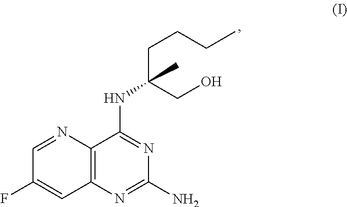

or a pharmaceutically acceptable salt thereof, wherein the compound of Formula I is administered orally once a week for 24 weeks starting at week 12 while the subject is fasting,
200 mg of a dsRNA of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3′-phosphate, each u is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and wherein the dsRNA is administered by subcutaneous injection once every 4 weeks for 24 weeks starting at day 1,
0.3 mg/kg of nivolumab, wherein the nivolumab is administered by intravenous injection once every 4 weeks for 24 weeks starting at week 12, and
25 mg of a compound of Formula (II):

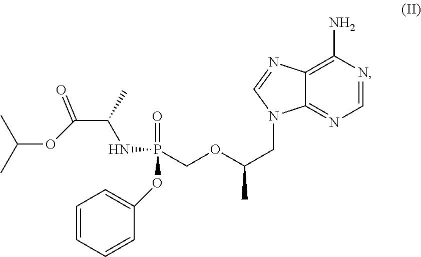 [SMILES: CC(C)OC(=O)[C@H](C)N[P@](=O)(CO[C@H](C)Cn1cnc2c(N)ncnc21)Oc1ccccc1]

wherein the compound of Formula II is administered orally once daily for 36 weeks starting at day 1,
wherein following completion of treatment, the subject is characterized by
   (i) a hepatitis B viral load of less than about 20 international units per milliliter (IU/mL);
   (ii) negative for the hepatitis B e-antigen (HBeAg); and
   (iii) negative for the hepatitis B surface antigen (HBsAg),
thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising
3 mg of a compound of Formula (I):

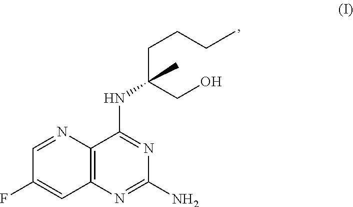

or a pharmaceutically acceptable salt thereof, wherein the compound of Formula I is administered orally once a week for 24 weeks starting at week 12 while the subject is fasting,
200 mg of a dsRNA of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3′-phosphate, each u is 2′-O-methyluridine-3′-phosphate, each Af is 2′-fluoroadenosine-3′-phosphate, each Cf is 2′-fluorocytidine-3′-phosphate, each (if is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and wherein the dsRNA is administered by subcutaneous injection once every 4 weeks for 24 weeks starting at day 1,
0.3 mg/kg of nivolumab, wherein the nivolumab is administered by intravenous injection once every 4 weeks for 24 weeks starting at week 12, and
25 mg of a compound of Formula (II):

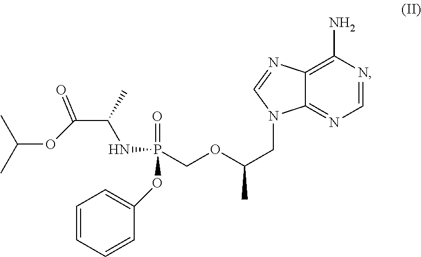 [SMILES: CC(C)OC(=O)[C@H](C)N[P@](=O)(CO[C@H](C)Cn1cnc2c(N)ncnc21)Oc1ccccc1]

wherein the compound of Formula II is administered orally once daily for 36 weeks starting at day 1,
wherein following completion of treatment, the subject is characterized by
   (i) a hepatitis B viral load of less than the lower limit of quantitation;
   (ii) negative for the hepatitis B e-antigen (HBeAg);
   (iii) alanine aminotransferase less than about twice the upper limit of normal; and
   (iv) negative for the hepatitis B surface antigen (HBsAg),
thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising
3 mg of a compound of Formula (I):

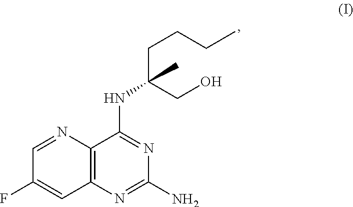

or a pharmaceutically acceptable salt thereof, wherein the compound of Formula I is administered orally once a week for 24 weeks starting at week 12 while the subject is fasting,
200 mg of a dsRNA of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein the dsRNA is administered by subcutaneous injection once every 4 weeks for 24 weeks starting at day 1,
0.3 mg/kg nivolumab, wherein the nivolumab is administered by intravenous injection once every 4 weeks for 24 weeks starting at week 12, and
25 mg of a compound of Formula (II):

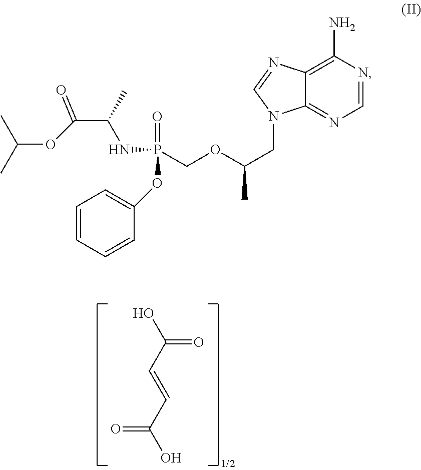

wherein the compound of Formula II is administered orally once daily for 36 weeks starting at day 1,
wherein following completion of treatment, the subject is characterized by
   (i) a hepatitis B viral load of less than about 20 international units per milliliter (IU/mL);
   (ii) negative for the hepatitis B e-antigen (HBeAg); and
   (iii) negative for the hepatitis B surface antigen (HBsAg),
thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising
3 mg of a compound of Formula (I):

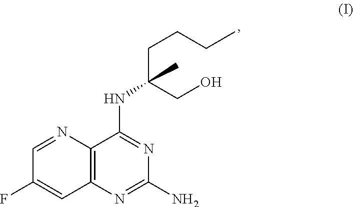

or a pharmaceutically acceptable salt thereof, wherein the compound of Formula I is administered orally once a week for 24 weeks starting at week 12 while the subject is fasting,
200 mg of a dsRNA of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein the dsRNA is administered by subcutaneous injection once every 4 weeks for 24 weeks starting at day 1,
0.3 mg/kg nivolumab, wherein the nivolumab is administered by intravenous injection once every 4 weeks for 24 weeks starting at week 12, and
25 mg of a compound of Formula (II):

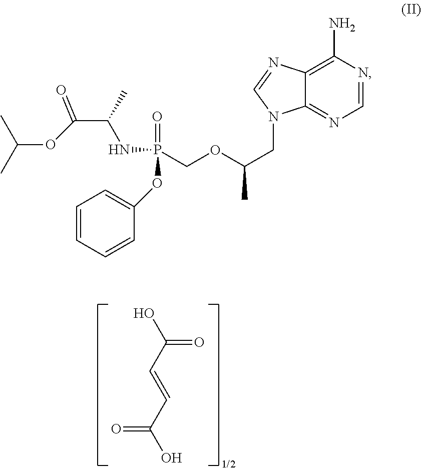

wherein the compound of Formula II is administered orally once daily for 36 weeks starting at day 1,
wherein following completion of treatment, the subject is characterized by
   (i) a hepatitis B viral load of less than the lower limit of quantitation;
   (ii) negative for the hepatitis B e-antigen (HBeAg);
   (iii) alanine aminotransferase less than about twice the upper limit of normal; and
   (iv) negative for the hepatitis B surface antigen (HBsAg),
thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising
3 mg of a compound of Formula (I):

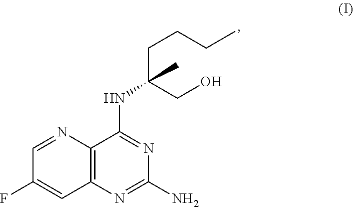

or a pharmaceutically acceptable salt thereof, wherein the compound of Formula I is administered orally once a week for 24 weeks starting at week 12 while the subject is fasting,
200 mg of a dsRNA of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3′-phosphate, each a is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and wherein the dsRNA is administered by subcutaneous injection once every 4 weeks for 24 weeks starting at day 1,
0.3 mg/kg nivolumab, wherein the nivolumab is administered by intravenous injection once every 4 weeks for 24 weeks starting at week 12, and
25 mg of a compound of Formula (II):

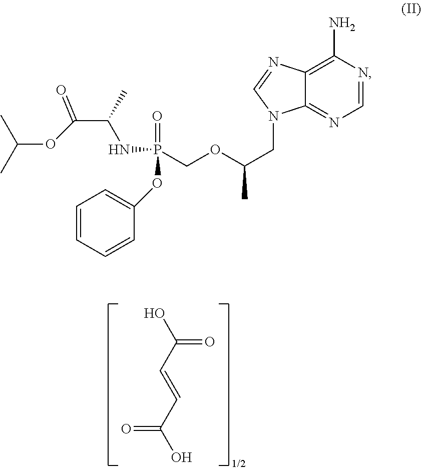

wherein the compound of Formula II is administered orally once daily for 36 weeks starting at day 1,
wherein following completion of treatment, the subject is characterized by
   (i) a hepatitis B viral load of less than about 20 international units per milliliter (IU/mL);
   (ii) negative for the hepatitis B e-antigen (HBeAg); and
   (iii) negative for the hepatitis B surface antigen (HBsAg),
thereby treating and/or preventing the hepatitis B viral infection in the subject.

In some embodiments, provided herein is a method of treating and/or preventing a hepatitis B viral infection in a subject in need thereof, comprising administering to the subject a therapeutically effective amount of a combination therapy regimen comprising
3 mg of a compound of Formula (I):

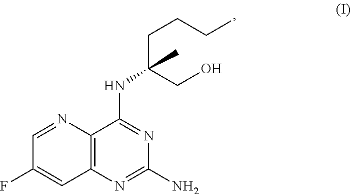

or a pharmaceutically acceptable salt thereof, wherein the compound of Formula I is administered orally once a week for 24 weeks starting at week 12 while the subject is fasting,
200 mg of a dsRNA of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2-O-methylguanosine-3′-phosphate, each u is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and wherein the dsRNA is administered by subcutaneous injection once every 4 weeks for 24 weeks starting at day 1,
0.3 mg/kg nivolumab, wherein the nivolumab is administered by intravenous injection once every 4 weeks for 24 weeks starting at week 12, and
25 mg of a compound of Formula (II):

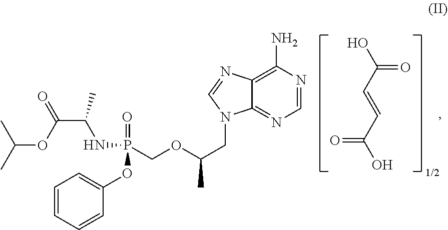

wherein the compound of Formula II is administered orally once daily for 36 weeks starting at day 1,
wherein following completion of treatment, the subject is characterized by
   (i) a hepatitis B viral load of less than the lower limit of quantitation;
   (ii) negative for the hepatitis B e-antigen (HBeAg);
   (iii) alanine aminotransferase less than about twice the upper limit of normal; and
   (iv) negative for the hepatitis B surface antigen (HBsAg),
thereby treating and/or preventing the hepatitis B viral infection in the subject.

In some embodiments, the present disclosure provides a method for manufacturing a medicament for treating and/or preventing a hepatitis B viral infection in a subject in need thereof, characterized in that a therapeutically effective amount of a combination therapy regimen comprising
a compound of Formula (I):

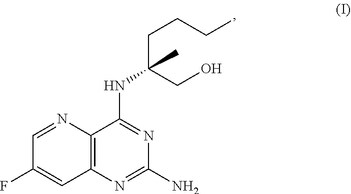

or a pharmaceutically acceptable salt thereof,
a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and
a PD-1/PD-L1 inhibitor, or a pharmaceutically acceptable salt thereof, is used.

In some embodiments, the present disclosure provides a method for manufacturing a medicament for treating and/or preventing a hepatitis B viral infection in a subject in need thereof, characterized in that a therapeutically effective amount of a combination therapy regimen comprising
a compound of Formula (I):

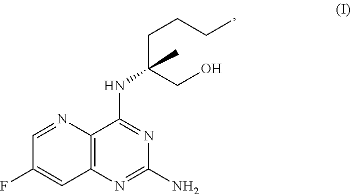

or a pharmaceutically acceptable salt thereof,
a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3′-phosphate, each u is 2′-O-methyluridine-3′-phosphate, each Af is 2′-fluoroadenosine-3′-phosphate, each Cf is 2′-fluorocytidine-3′-phosphate, each Gf is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and
a PD-1/PD-L1 inhibitor, or a pharmaceutically acceptable salt thereof, is used.

In some embodiments, the present disclosure provides use of a therapeutically effective amount of a combination therapy regimen comprising
a compound of Formula (I):

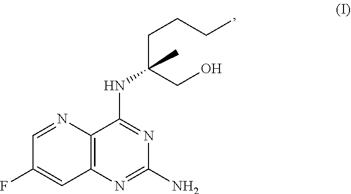

or a pharmaceutically acceptable salt thereof,
a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and
a PD-1/PD-L1 inhibitor, or a pharmaceutically acceptable salt thereof, for the manufacture of a medicament for the treatment and/or prevention of a hepatitis
   B viral infection in a subject in need thereof.

In some embodiments, the present disclosure provides use of a therapeutically effective amount of a combination therapy regimen comprising
a compound of Formula (I):

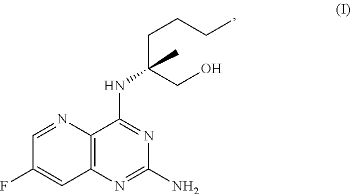

or a pharmaceutically acceptable salt thereof,
a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3′-phosphate, each a is 2′-O-methyluridine-3′-phosphate, each Af is 2′-fluoroadenosine-3′-phosphate, each Cf is 2′-fluorocytidine-3′-phosphate, each (if is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and
a PD-1/PD-L1 inhibitor, or a pharmaceutically acceptable salt thereof,
for the manufacture of a medicament for the treatment and/or prevention of a hepatitis B viral infection in a subject in need thereof.

In some embodiments, the present disclosure provides a combination therapy regimen comprising
a compound of Formula (I):

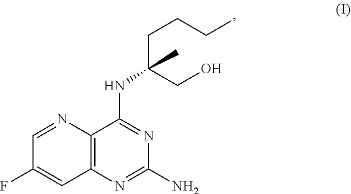

or a pharmaceutically acceptable salt thereof,
a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and
a PD-1/PD-L1 inhibitor, or a pharmaceutically acceptable salt thereof,
for use in the treatment and/or prevention of a hepatitis B viral infection in a subject in need thereof.

In some embodiments, the present disclosure provides a combination therapy regimen comprising
a compound of Formula (I):

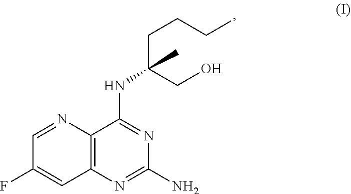

or a pharmaceutically acceptable salt thereof,
a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, wherein each a is 2′-O-methyladenosine-3′-phosphate, each c is 2′-O-methylcytidine-3′-phosphate, each g is 2′-O-methylguanosine-3′-phosphate, each u is 2′-O-methyluridine-3′-phosphate, each Af is 2′-fluoroadenosine-3′-phosphate, each Cf is 2′-fluorocytidine-3′-phosphate, each (if is 2′-fluoroguanosine-3′-phosphate, Uf is 2′-fluorouridine-3′-phosphate, (Agn) is adenosine-glycol nucleic acid (GNA), each s is a phosphorothioate linkage, and L96 is N-[tris(GalNAc-alkyl)-amidodecanoyl)]-4-hydroxyprolinol, and
a PD-1/PD-L1 inhibitor, or a pharmaceutically acceptable salt thereof, for use in the treatment and/or prevention of a hepatitis B viral infection in a subject in need thereof.

### IV. Examples

### Example 1. Study to Evaluate the Safety and Efficacy of Selgantolimod Combination

Therapies for Treatment of Chronic Hepatitis B (CHB) Study Objectives

The primary objectives of this study are as follows:
To evaluate the safety and tolerability of study treatment(s)
To evaluate the efficacy of study treatment(s) as measured by the proportion of subjects who achieve functional cure, defined as negative qualitative hepatitis B surface antigen (HBsAg loss) and hepatitis B virus (HBV) DNA<20 IU/mL at Follow-Up (FU) Week 24

The secondary objectives of this study are as follows:
To evaluate the proportion of subjects with HBsAg loss with and without anti-HBsAg seroconversion during the study.
To evaluate in subjects with CHB who are hepatitis B e antigen (HBeAg)-positive at baseline, the proportion of subjects who achieve HBeAg loss with and without anti-HBeAg seroconversion during the study.
To evaluate the proportion of subjects who remain off nucleos(t)ide(s) (NUC) treatment during FU.
To evaluate the proportion of subjects experiencing HBV virologic breakthrough during study treatment(s).
   The exploratory objectives of this study are as follows:
To evaluate the change from baseline in quantitative HBV RNA, HBV DNA (including digital droplet polymerase chain reaction [ddPCR] if available), hepatitis B core-related antigen (HBcrAg), HBeAg, HBsAg, and glycosylated fraction of HBsAg (if applicable) during and after study treatment(s) discontinuation.
To evaluate the effect of study treatment(s) on peripheral cytokine activation and immune response.
To characterize the relationship between immunologic changes and circulating HBV viral markers.
To identify or validate host and/or viral genetic markers that may be predictive of the natural history of disease, response to therapy and/or tolerability of medical therapies through genetic discovery research (eg, pharmacogenomics [PG]), in subjects who provide their separate and specific consent.
To characterize the pharmacokinetics (PK) of SLGN in combination with VIR-2218 and nivolumab.
To characterize HBV viral variants present at baseline and/or emerged during treatment that may be associated with response to the study treatment(s).

### Study Design

This is a Phase 2, open-label study to evaluate the safety and efficacy of SLGN-containing combination therapies in chronic hepatitis B (CHB) subjects. The study will consist of 3 cohorts (Cohorts 1, 2, and 3). Approximately 40 NUC-suppressed and 80 viremic CHB-infected subjects, may be enrolled and assigned into a cohort below. Each cohort will enroll an approximate (±10%) equal number of HBeAg positive and negative subjects; and up to 20% of subjects can have HBsAg<100 IU/mL

NUC-suppressed Cohort. Cohort 1 (n=40):
Tenofovir alafenamide (TAF) 25 mg tablet administered orally once daily for 36 weeks
VIR-2218 200 mg administered as subcutaneous (SC) injection once every 4 weeks for 24 weeks (total 6 doses)
   At Week 12, add-on and initiate the below treatment:
SLGN 3 mg (2×1.5-mg tablets) administered orally while fasting once a week on the same day for 24 weeks (total 24 doses)
Nivolumab 0.3 mg/kg administered intravenously (IV) once every 4 weeks for 24 weeks (total 6 doses)

### Viremic Cohorts (Cohorts 2 and 3).

Cohort 2. Subjects will be randomized 2:1 into Cohort 2 Groups A and B and stratified by HBsAg> or ≤3_{log 10} IU/mL.

Group A (n=40):
VIR-2218 200 mg administered as SC injection once every 4 weeks for 24 weeks (total 6 doses)
   At Week 12, add-on and initiate the below treatment:
SLGN 3 mg (2×1.5-mg-tablets) administered orally while fasting once a week on the same day for 24 weeks (total 24 doses)
Nivolumab 0.3 mg/kg administered IV once every 4 weeks for 24 weeks (total 6 doses)

Group B (n=20):
SLGN 3 mg (2×1.5-mg-tablets) administered orally while fasting once a week on the same day for 24 weeks (total 24 doses)
Nivolumab 0.3 mg/kg administered IV once every 4 weeks for 24 weeks (total 6 doses)

Cohort 3 (n=20). Cohort 3 will be initiated at the discretion of the sponsor after Cohort 2 has completed enrollment.
VIR-2218 200 mg administered as SC injection once every 4 weeks for 24 weeks (total 6 doses)
SLGN 3 mg (2×1.5-mg tablets) administered orally while fasting once a week on the same day for 24 weeks (total 24 doses)
Nivolumab 0.3 mg/kg administered IV once every 4 weeks for 24 weeks (total 6 doses)

Follow-Up Period. At the end of treatment, all subjects will enter a FU period.
All subjects not on TAF treatment at end of treatment (EOT) will enter a treatment-free follow up (TFFU) period
Subjects who are on TAF treatment and meet the criteria below at the EOT visit will stop all treatments, no later than FU Week 1 visit, and enter a TFFU period: (1) HBV DNA<20 IU/mL; (2) HBeAg negative; and (3)−HBsAg≤100 IU/mL.
All remaining subjects will continue on TAF or other NUC treatment and enter a FU period.

Subjects who do not meet the above criteria but choose to discontinue NUC at EOT can do so with medical monitor approval.

Number of Subjects Planned: Approximately 120 subjects

Target Population: Adult, noncirrhotic, subjects with CHB infection who are viremic or virally suppressed on a commercially approved HBV NUC treatment.

Duration of Treatment:
TAF 25 mg tablet will be administered orally once daily for up to 84 weeks, as applicable, in Cohort 1
VIR-2218 200 mg SC will be administered once every 4 weeks for 24 weeks (6 doses)
Nivolumab 0.3 mg/kg IV will be administered once every 4 weeks for 24 weeks (6 doses)
SLGN 3 mg (2×1.5-mg tablets) will be administered orally while fasting once a week on the same day for 24 weeks (total 24 doses)
Male and nonpregnant female subjects, ages 18 to 65 years, inclusive, with chronic HBV infection without the presence of cirrhosis, and who are viremic or virally suppressed on NUC for at least 6 months may be eligible for the study.

Study Procedures/Frequency. After consent is obtained, screening assessments will be completed within 30 days prior to the Baseline/Day 1 treatment, screening window can be extended to 45 days with sponsor approval. All subjects will complete the following study treatments below. Subjects who remain on NUC into FU period are not required to attend FU Weeks 2 and 8 visits.

### Cohort 1:

Screening Visit
Treatment Period Visits: Baseline/Day 1, Weeks 4, 8, 12, 13, 14, 16, 20, 24, 28, 32, and 36
FU Visits: Weeks 1, 2, 4, 8, 12, 16 (for women of childbearing potential), 24 (Primary), 36, and 48

### Cohort 2. Group A:

Treatment Period Visits: Baseline/Day 1, Weeks 4, 8, 12, 13, 14, 16, 20, 24, 28, 32, and 36
FU Visits: Weeks 1, 2, 4, 8, 12, 16 (for women of childbearing potential), 24 (Primary), 36, and 48

### Cohort 2. Group B:

Treatment Period Visits: Baseline/Day 1, Weeks 1, 2, 4, 8, 12, 14, 16, 20, and 24
FU Visits: Weeks 1, 2, 4, 8, 12, 16 (for women of childbearing potential), 24 (Primary), 36, and 48

### Cohort 3.

Treatment Period Visits: Baseline/Day 1, Weeks 1, 2, 4, 8, 12, 14, 16, 20, and 24
FU Visits: Weeks 1, 2, 4, 8, 12, 16 (for women of childbearing potential), 24 (Primary), 36, and 48.

Test Product, Dose, and Mode of Administration: Selgantolimod will be supplied as tablets in strengths of 1.5 mg. SLGN 3 mg (2×1.5-mg tablets) will be administered while fasting, once a week, on the same day. Subjects must be fasting for at least 8 hours overnight (no food or drinks, except water) and continue through the morning, with no food or drinks, including water, 1 hour before to 2 hours after dosing. After 2 hours post dose, water is allowed and after 4 hours post dose, subjects are allowed food and drinks. Subjects should take their other prescribed medications, including NUC treatment, no earlier than 2 hours after SLGN dosing or, if medications require dosing with food, no earlier than 4 hours after SLGN dosing.

### Study Endpoints

The primary endpoint of this study is as follows:
The proportion of subjects who achieve functional cure, defined as HBsAg loss and HBV DNA<20 IU/mL at FU Week 24.

The secondary endpoints of this study are as follows:
The proportion of subjects with HBsAg loss with and without anti-HBsAg seroconversion during the study.
The proportion of subjects with HBeAg loss with and without anti-HBeAg seroconversion during the study in subjects with CHB who are HBeAg-positive at baseline.
The proportion of subjects who remain off NUC treatment during FU.
The proportion of subjects experiencing HBV virologic breakthrough (defined as HBV DNA≥69 IU/mL for 2 consecutive visits after having been<20 IU/mL or confirmed HBV DNA≥1 log₁₀ IU/mL increase from nadir) during study treatment(s).

### Study Design

This is an open-label study to evaluate the safety and efficacy of SLGN-containing combination therapies in subjects with CHB. Approximately 40 NUC-suppressed and 80 viremic CHB-infected subjects, may be enrolled and assigned into a cohort below. Each cohort will enroll an approximate (±10%) equal number of HBeAg-positive and HBeAg-negative subjects; and up to 20% of subjects can have HBsAg≤100 IU/mL.

### Study Treatments

NUC-suppressed Cohort. Cohort 1 (n=40):
TAF 25 mg tablet administered orally once daily for 36 weeks
VIR-2218 200 mg administered via SC injection once every 4 weeks for 24 weeks (total 6 doses)
   At Week 12, add-on and initiate the below treatment:
SLGN 3 mg (2×1.5-mg tablets) administered orally while fasting once a week on the same day for 24 weeks (total 24 doses)
Nivolumab 0.3 mg/kg administered intravenously (IV) once every 4 weeks for 24 weeks (total 6 doses)

### Viremic Cohorts (Cohorts 2 and 3).

Cohort 2. Subjects will be randomized 2:1 into Cohort 2 (Groups A and B) and stratified by HBsAg> or ≤3 log₁₀ IU/mL.

Group A (n=40):
VIR-2218 200 mg administered via SC injection once every 4 weeks for 24 weeks (total 6 doses)
   At Week 12, add-on and initiate the below treatment:
SLGN 3 mg (2×1.5-mg tablets) administered orally while fasting once a week on the same day for 24 weeks (total 24 doses)
Nivolumab 0.3 mg/kg administered IV once every 4 weeks for 24 weeks (total 6 doses)
   Group B (n=20):
SLGN 3 mg (2×1.5-mg tablets) administered orally while fasting once a week on the same day for 24 weeks (total 24 doses)
Nivolumab 0.3 mg/kg administered IV once every 4 weeks for 24 weeks (total 6 doses)

Cohort 3 (n=20). Cohort 3 will be initiated at the discretion of the sponsor after Cohort 2 has completed enrollment.
VIR-2218 200 mg administered via SC injection once every 4 weeks for 24 weeks (total 6 doses)
SLGN 3 mg (2×1.5 mg-tablets) administered orally while fasting once a week on the same day for 24 weeks (total 24 doses)
Nivolumab 0.3 mg/kg administered IV once every 4 weeks for 24 weeks (total 6 doses)

### Duration of Treatment

The duration of study treatment are as follows:
TAF 25 mg tablet will be administered orally once daily up to 84 weeks, as applicable, in Cohort 1
VIR-2218 200 mg SC will be administered once every 4 weeks for 24 weeks (6 doses)
Nivolumab 0.3 mg/kg IV will be administered once every 4 weeks for 24 weeks (6 doses)
SLGN 3 mg (2×1.5-mg tablets) will be administered orally while fasting once a week on the same day for 24 weeks (total 24 doses).
   After completing study treatments all subjects will undergo 48 weeks of FU.

### Study Follow-Up

At the end of treatment, all subjects will enter the FU period.
All subjects not on TAF treatment at end-of-treatment (EOT) will enter a TFFU period
Subjects who are on TAF treatment and meet the criteria below at the EOT visit will stop all treatments, no later than FU Week 1 visit, and enter a TFFU period: (1) HBV DNA<20 IU/mL; (2) HBeAg negative; and (3) HBsAg≤100 IU/mL
All remaining subjects will continue on TAF or other treatment and enter the FU period

Subjects who do not meet the above criteria but choose to discontinue NUC at EOT can do so with medical monitor approval.

### Number of Subjects and Subject Selection.

Cohorts 1-3 will enroll approximately 120 male and nonpregnant female subjects, ages 18 to 65 years, inclusive, with CHB infection without the presence of cirrhosis, and who are viremic or virally suppressed on NUC for at least 6 months.

Subjects in Cohort 1 should meet the following additional criteria to be eligible to participate in this study:
Have been on a commercially available HBV NUC treatment(s) (i.e., TAF, TDF, entecavir, adefovir, lamivudine, telbivudine, either as single agents or in combination) with no change in regimen for 3 months prior to screening and willing to initiate TAF 25 mg.
Have a historic HBV DNA<69 IU/mL, measured at least once at local laboratory, 6 or more months prior to screening.
HBV DNA<20 IU/mL by central laboratory at screening

Subjects in Cohort 2 and 3 should meet the following additional criteria at screening to be eligible to participate in this study:
HBV DNA>2000 IU/mL (HBeAg-negative) and HBV DNA>20,000 IU/mL (HBeAg-positive)

### Formulation

Selgantolimod. Selgantolimod tablets, 1.5 mg, have been formulated with microcrystalline cellulose, mannitol, croscarmellose sodium, and magnesium stearate. Tablets are round, plain-faced, film-coated and white. The white tablet film-coating contains polyvinyl alcohol, titanium dioxide, polyethylene glycol (PEG) 3350, and talc.

Nivolumab. Commercially available product of nivolumab injection will be used for this study. Further information regarding formulation is available in the current approved product label for nivolumab.

Tenofovir Alafenamide. Each film-coated tablet contains tenofovir alafenamide fumarate equivalent to 25 mg of TAF and have been formulated with croscarmellose sodium, lactose monohydrate, magnesium stearate, and microcrystalline cellulose. The tablets are yellow, round, film-coated, and debossed with “GSI” on one side of the tablet and “25” on the other side of the tablet.

VIR-2218. VIR-2218 is a clear, colorless to pale yellow solution, which will be supplied by the sponsor as a sterile solution for SC injection at a free acid concentration of 200 mg/mL.

### Dosage and Administration of Selgantolimod, Tenofovir, VIR-2218 and Nivolumab

Selgantolimod. Selgantolimod will be supplied as tablets in strengths of 1.5 mg. SLGN 3 mg (2×1.5-mg tablets) will be administered fasting once a week, on the same day. Subjects must be fasting for at least 8 hour overnight (no food or drinks, except water) and continue through the morning, with no food or drinks, including water, 1 hour before to 2 hours after dosing. After 2 hours post dose, water is allowed and after 4 hours post dose, subjects are allowed food and drinks. Subjects should take their other prescribed medications, including NUC treatment, no earlier than 2 hours after SLGN dosing or, if medications require dosing with food, no earlier than 4 hours after SLGN dosing.

Nivolumab. Nivolumab (Opdivo®) 40 mg/4 mL solution for injection will be supplied as single dose vials. Nivolumab 0.3 mg/kg will be administered as IV infusion over 30 minutes.

Tenofovir Alafenamide. TAF 25-mg tablet orally once daily with food.

VIR-2218. VIR-2218, 200 mg/mL, solution for injection will be supplied as 0.5 mL single dose vials. VIR-2218 200 mg (2×0.5 mL solution) will be administered subcutaneously.

### Randomization

Viremic Cohort 2 (Group A and B) Only. Subjects will be randomized 2:1 into Cohort 2 Groups A and B and stratified by HBsAg> or <3 log₁₀ IU/mL.

### Treatment Schedule

### Cohort 1:

Baseline/Day 1, Week 4 and Week 8—SC VIR-2218
Week 12, 16, and 20—SC VIR-2218; add-on IV nivolumab
Week 24, 28 and 32—IV Nivolumab
Oral TAF dose daily for 36 weeks
Oral SLGN dose weekly for 24 weeks, starting Week 12 through Week 35

### Cohort 2, Group A:

Baseline/Day 1, Weeks 4 and 8—SC VIR-2218
Weeks 12, 16, and 20—SC VIR-2218; add-on IV nivolumab
Weeks 24, 28 and 32—IV nivolumab
Oral SLGN dose weekly for 24 weeks, starting Week 12 through Week 35

Cohort 2, Group B:
Baseline/Day 1, Weeks 4, 8, 12, 16 and 20—IV nivolumab
Oral SLGN dose weekly for 24 weeks, starting Day 1 through Week 23

### Cohort 3:

Baseline/Day 1, and Weeks 4, 8, 12, 16, and 20—SC VIR-2218, IV nivolumab
Oral SLGN dose weekly for 24 weeks, starting Day 1 through Week 23

### Example 2. Study to Evaluate the Safety and Efficacy of Selgantolimod Combination Therapies for Treatment of Chronic Hepatitis B (CHB)

### Study Objectives

The primary objectives of this study are as follows:
To evaluate the safety and tolerability of study treatment(s)
To evaluate the efficacy of study treatment(s) as measured by the proportion of subjects who achieve functional cure, defined as negative qualitative hepatitis B surface antigen (HBsAg loss) and hepatitis B virus (HBV) DNA<lower limit of quantitation (LLOQ) at Follow-Up (FU) Week 24

The secondary objectives of this study are as follows:
To evaluate the proportion of subjects with HBsAg loss with and without anti-HBsAg seroconversion during the study.
To evaluate in subjects with CHB who are hepatitis B e antigen (HBeAg)-positive at baseline, the proportion of subjects who achieve HBeAg loss with and without anti-HBeAg seroconversion during the study.
To evaluate the proportion of subjects who remain off nucleos(t)ide(s) (NUC) treatment during FU.
To evaluate the proportion of subjects experiencing HBV virologic breakthrough during study treatment(s).
   The exploratory objectives of this study are as follows:
To evaluate the change from baseline in quantitative HBV RNA, HBV DNA (including digital droplet polymerase chain reaction [ddPCR] if available), hepatitis B core-related antigen (HBcrAg), HBeAg, HBsAg, and glycosylated fraction of HBsAg (if applicable) during and after study treatment(s) discontinuation.
To evaluate the effect of study treatment(s) on peripheral cytokine activation and immune response.
To characterize the relationship between immunologic changes and circulating HBV viral markers.
To identify or validate host and/or viral genetic markers that may be predictive of the natural history of disease, response to therapy and/or tolerability of medical therapies through genetic discovery research (eg, pharmacogenomics [PG]), in subjects who provide their separate and specific consent.
To characterize the pharmacokinetics (PK) of SLGN in combination with VIR-2218 and nivolumab.
To characterize HBV viral variants present at baseline and/or emerged during treatment that may be associated with response to the study treatment(s).

### Study Design

This is a Phase 2, open-label study to evaluate the safety and efficacy of SLGN-containing combination therapies in chronic hepatitis B (CHB) subjects. The study will consist of 3 cohorts (Cohorts 1, 2, and 3). Approximately 40 NUC-suppressed and 80 viremic CHB-infected subjects, may be enrolled and assigned into a cohort below. Each cohort will enroll a minimum of 20% HBeAg positive subjects; and up to 20% of subjects can have HBsAg≤100 IU/mL

NUC-suppressed Cohort. Cohort 1 (n=40):
Tenofovir alafenamide (TAF) 25 mg tablet administered orally once daily for 36 weeks
VIR-2218 200 mg administered as subcutaneous (SC) injection once every 4 weeks for 24 weeks (total 6 doses)
   At Week 12, add-on and initiate the below treatment:
SLGN 3 mg (2×1.5-mg tablets) administered orally while fasting once a week on the same day for 24 weeks (total 24 doses)
Nivolumab 0.3 mg/kg administered intravenously (IV) once every 4 weeks for 24 weeks (total 6 doses)

### Viremic Cohorts (Cohorts 2 and 3).

Cohort 2. Subjects will be randomized 2:1 into Cohort 2 Groups A and B and stratified by HBsAg> or ≤3_{log 10} IU/mL.

Group A (n=40):
VIR-2218 200 mg administered as SC injection once every 4 weeks for 24 weeks (total 6 doses)
   At Week 12, add-on and initiate the below treatment:
SLGN 3 mg (2×1.5-mg-tablets) administered orally while fasting once a week on the same day for 24 weeks (total 24 doses)
Nivolumab 0.3 mg/kg administered IV once every 4 weeks for 24 weeks (total 6 doses)

Group B (n=20):
SLGN 3 mg (2×1.5-mg-tablets) administered orally while fasting once a week on the same day for 24 weeks (total 24 doses)
Nivolumab 0.3 mg/kg administered IV once every 4 weeks for 24 weeks (total 6 doses)

Cohort 3 (n=20). Cohort 3 will be initiated at the discretion of the sponsor after Cohort 2 has completed enrollment.
VIR-2218 200 mg administered as SC injection once every 4 weeks for 24 weeks (total 6 doses)
SLGN 3 mg (2×1.5-mg tablets) administered orally while fasting once a week on the same day for 24 weeks (total 24 doses)
Nivolumab 0.3 mg/kg administered IV once every 4 weeks for 24 weeks (total 6 doses)

Follow-Up Period. At the end of treatment, all subjects will enter a FU period.
All subjects not on TAF treatment at end of treatment (EOT) will enter a treatment-free follow up (TFFU) period
Subjects who are on TAF treatment and meet the criteria below at the EOT visit will stop all treatments, no later than FU Week 1 visit, and enter a TFFU period: (1) HBV DNA<LLOQ; (2) HBeAg negative; and (3)−HBsAg≤100 IU/mL.
All remaining subjects will continue on TAF or other NUC treatment and enter a FU period.

Subjects who do not meet the above criteria but choose to discontinue NUC at EOT can do so with medical monitor approval.

Number of Subjects Planned: Approximately 120 subjects

Target Population: Adult, noncirrhotic, subjects with CHB infection who are viremic or virally suppressed on a commercially approved HBV NUC treatment.

### Duration of Treatment:

TAF 25 mg tablet will be administered orally once daily for up to 84 weeks, as applicable, in Cohort 1
VIR-2218 200 mg SC will be administered once every 4 weeks for 24 weeks (6 doses)
Nivolumab 0.3 mg/kg IV will be administered once every 4 weeks for 24 weeks (6 doses)
SLGN 3 mg (2×1.5-mg tablets) will be administered orally while fasting once a week on the same day for 24 weeks (total 24 doses)
Male and nonpregnant female subjects, ages 18 to 65 years, inclusive, with chronic HBV infection without the presence of cirrhosis, and who are viremic or virally suppressed on NUC for at least 6 months may be eligible for the study.

Study Procedures/Frequency. After consent is obtained, screening assessments will be completed within 45 days prior to the Baseline/Day 1 treatment. All subjects will complete the following study treatments below. Subjects who remain on NUC into FU period are not required to attend FU Weeks 2 and 8 visits.

### Cohort 1:

Screening Visit
Treatment Period Visits: Baseline/Day 1, Weeks 4, 8, 12, 13, 14, 16, 20, 24, 28, 32, and 36
FU Visits: Weeks 1, 2, 4, 8, 12, 16 (for women of childbearing potential), 24 (Primary), 36, and 48

### Cohort 2. Group A:

Treatment Period Visits: Baseline/Day 1, Weeks 4, 8, 12, 13, 14, 16, 20, 24, 28, 32, and 36
FU Visits: Weeks 1, 2, 4, 8, 12, 16 (for women of childbearing potential), 24 (Primary), 36, and 48

### Cohort 2. Group B:

Treatment Period Visits: Baseline/Day 1, Weeks 1, 2, 4, 8, 12, 14, 16, 20, and 24
FU Visits: Weeks 1, 2, 4, 8, 12, 16 (for women of childbearing potential), 24 (Primary), 36, and 48

### Cohort 3.

Treatment Period Visits: Baseline/Day 1, Weeks 1, 2, 4, 8, 12, 14, 16, 20, and 24
FU Visits: Weeks 1, 2, 4, 8, 12, 16 (for women of childbearing potential), 24 (Primary), 36, and 48.

Test Product, Dose, and Mode of Administration: Selgantolimod will be supplied as tablets in strengths of 1.5 mg. SLGN 3 mg (2×1.5-mg tablets) will be administered while fasting, once a week, on the same day. Subjects must be fasting for at least 8 hours overnight (no food or drinks, except water) and continue through the morning, with no food or drinks, including water, 1 hour before to 2 hours after dosing. After 2 hours post dose, water is allowed and after 4 hours post dose, subjects are allowed food and drinks. Subjects should take their other prescribed medications, including NUC treatment, no earlier than 2 hours after SLGN dosing or, if medications require dosing with food, no earlier than 4 hours after SLGN dosing.

### Study Endpoints

The primary endpoint of this study is as follows:
The proportion of subjects who achieve functional cure, defined as HBsAg loss and HBV DNA<LLOQ at FU Week 24.

The secondary endpoints of this study are as follows:
The proportion of subjects with HBsAg loss with and without anti-HBsAg seroconversion during the study.
The proportion of subjects with HBeAg loss with and without anti-HBeAg seroconversion during the study in subjects with CHB who are HBeAg-positive at baseline.
The proportion of subjects who remain off NUC treatment during FU.
The proportion of subjects experiencing HBV virologic breakthrough (defined as HBV DNA≥69 IU/mL for 2 consecutive visits after having been<LLOQ or confirmed HBV DNA≥1 log₁₀ IU/mL increase from nadir) during study treatment(s).

### Study Design

This is an open-label study to evaluate the safety and efficacy of SLGN-containing combination therapies in subjects with CHB. Approximately 40 NUC-suppressed and 80 viremic CHB-infected subjects, may be enrolled and assigned into a cohort below. Each cohort will enroll a minimum of 20% HBeAg-positive subjects; and up to 20% of subjects can have HBsAg≤100 IU/mL.

### Study Treatments

NUC-suppressed Cohort. Cohort 1 (n=40):
TAF 25 mg tablet administered orally once daily for 36 weeks
VIR-2218 200 mg administered via SC injection once every 4 weeks for 24 weeks (total 6 doses)
   At Week 12, add-on and initiate the below treatment:
SLGN 3 mg (2×1.5-mg tablets) administered orally while fasting once a week on the same day for 24 weeks (total 24 doses)
Nivolumab 0.3 mg/kg administered intravenously (IV) once every 4 weeks for 24 weeks (total 6 doses)

### Viremic Cohorts (Cohorts 2 and 3).

Cohort 2. Subjects will be randomized 2:1 into Cohort 2 (Groups A and B) and stratified by HBsAg> or ≤3 log₁₀ IU/mL.

Group A (n=40):
VIR-2218 200 mg administered via SC injection once every 4 weeks for 24 weeks (total 6 doses)
   At Week 12, add-on and initiate the below treatment:
SLGN 3 mg (2×1.5-mg tablets) administered orally while fasting once a week on the same day for 24 weeks (total 24 doses)
Nivolumab 0.3 mg/kg administered IV once every 4 weeks for 24 weeks (total 6 doses)

Group B (n=20):
SLGN 3 mg (2×1.5-mg tablets) administered orally while fasting once a week on the same day for 24 weeks (total 24 doses)
Nivolumab 0.3 mg/kg administered IV once every 4 weeks for 24 weeks (total 6 doses)

Cohort 3 (n=20). Cohort 3 will be initiated at the discretion of the sponsor after Cohort 2 has completed enrollment.
VIR-2218 200 mg administered via SC injection once every 4 weeks for 24 weeks (total 6 doses)
SLGN 3 mg (2×1.5 mg-tablets) administered orally while fasting once a week on the same day for 24 weeks (total 24 doses)
Nivolumab 0.3 mg/kg administered IV once every 4 weeks for 24 weeks (total 6 doses)

### Duration of Treatment

The duration of study treatment are as follows:
TAF 25 mg tablet will be administered orally once daily up to 84 weeks, as applicable, in Cohort 1
VIR-2218 200 mg SC will be administered once every 4 weeks for 24 weeks (6 doses)
Nivolumab 0.3 mg/kg IV will be administered once every 4 weeks for 24 weeks (6 doses)
SLGN 3 mg (2×1.5-mg tablets) will be administered orally while fasting once a week on the same day for 24 weeks (total 24 doses).
   After completing study treatments all subjects will undergo 48 weeks of FU.

### Study Follow-Up

At the end of treatment, all subjects will enter the FU period.
All subjects not on TAF treatment at end-of-treatment (EOT) will enter a TFFU period
Subjects who are on TAF treatment and meet the criteria below at the EOT visit will stop all treatments, no later than FU Week 1 visit, and enter a TFFU period: (1) HBV DNA<LLOQ; (2) HBeAg negative; (3) Alanine aminotransferase (ALT)<2×upper limit of normal (ULN); and (4) Confirmed HBsAg≤100 IU/mL with repeat test
All remaining subjects will continue on TAF or other treatment and enter the FU period
HBeAg-positive subjects meeting all of the above criteria with the exception of HBeAg status may also discontinue NUC treatment upon agreement between the investigator and the sponsor's medical monitor.

Subjects who do not meet the above criteria but choose to discontinue NUC treatment at EOT may do so upon agreement between the investigator and the sponsor's medical monitor approval. Subjects who meet the above criteria but the investigator wants to continue NUC treatment at EOT may do so upon agreement between the investigator and the sponsor's medical monitor after discussion to evaluate risks and benefits.

### Number of Subjects and Subject Selection.

Cohorts 1-3 will enroll approximately 120 male and nonpregnant female subjects, ages 18 to 65 years, inclusive, with CHB infection without the presence of cirrhosis, and who are viremic or virally suppressed on NUC for at least 6 months.

Subjects in Cohort 1 should meet the following additional criteria to be eligible to participate in this study:
Have been on a commercially available HBV NUC treatment(s) (i.e., TAF, TDF, entecavir, adefovir, lamivudine, telbivudine, either as single agents or in combination) with no change in regimen for 3 months prior to screening and willing to initiate TAF 25 mg.
Have a historic HBV DNA<69 IU/mL, measured at least once at local laboratory, 6 or more months prior to screening.
HBV DNA<LLOQ by central laboratory at screening

Subjects in Cohort 2 and 3 should meet the following additional criteria at screening to be eligible to participate in this study:
HBV DNA>2000 IU/mL (HBeAg-negative) and HBV DNA>20,000 IU/mL (HBeAg-positive)

### Formulation

Selgantolimod. Selgantolimod tablets, 1.5 mg, have been formulated with microcrystalline cellulose, mannitol, croscarmellose sodium, and magnesium stearate. Tablets are round, plain-faced, film-coated and white. The white tablet film-coating contains polyvinyl alcohol, titanium dioxide, polyethylene glycol (PEG) 3350, and talc.

Nivolumab. Commercially available product of nivolumab injection will be used for this study. Further information regarding formulation is available in the current approved product label for nivolumab.

Tenofovir Alafenamide. Each film-coated tablet contains tenofovir alafenamide fumarate equivalent to 25 mg of TAF and have been formulated with croscarmellose sodium, lactose monohydrate, magnesium stearate, and microcrystalline cellulose. The tablets are yellow, round, film-coated, and debossed with “GSI” on one side of the tablet and “25” on the other side of the tablet.

VIR-2218. VIR-2218 is a clear, colorless to pale yellow solution, which will be supplied by the sponsor as a sterile solution for SC injection at a free acid concentration of 200 mg/mL.

### Dosage and Administration of Selgantolimod, Tenofovir, VIR-2218 and Nivolumab

Selgantolimod. Selgantolimod will be supplied as tablets in strengths of 1.5 mg. SLGN 3 mg (2×1.5-mg tablets) will be administered fasting once a week, on the same day. Subjects must be fasting for at least 8 hour overnight (no food or drinks, except water) and continue through the morning, with no food or drinks, including water, 1 hour before to 2 hours after dosing. After 2 hours post dose, water is allowed and after 4 hours post dose, subjects are allowed food and drinks. Subjects should take their other prescribed medications, including NUC treatment, no earlier than 2 hours after SLGN dosing or, if medications require dosing with food, no earlier than 4 hours after SLGN dosing.

Nivolumab. Nivolumab (Opdivo®) 40 mg/4 mL solution for injection will be supplied as single dose vials. Nivolumab 0.3 mg/kg will be administered as IV infusion over 45-60 minutes.

Tenofovir Alafenamide. TAF 25-mg tablet orally once daily with food.

VIR-2218. VIR-2218, 200 mg/mL, solution for injection will be supplied as 0.5 mL single dose vials. VIR-2218 200 mg (2×0.5 mL solution) will be administered subcutaneously.

### Randomization

Viremic Cohort 2 (Group A and B) Only. Subjects will be randomized 2:1 into Cohort 2 Groups A and B and stratified by HBsAg> or <3 log₁₀ IU/mL.

### Treatment Schedule

### Cohort 1:

Baseline/Day 1, Week 4 and Week 8—SC VIR-2218
Week 12, 16, and 20—SC VIR-2218; add-on IV nivolumab
Week 24, 28 and 32—IV Nivolumab
Oral TAF dose daily for 36 weeks
Oral SLGN dose weekly for 24 weeks, starting Week 12 through Week 35

### Cohort 2, Group A:

Baseline/Day 1, Weeks 4 and 8—SC VIR-2218
Weeks 12, 16, and 20—SC VIR-2218; add-on IV nivolumab
Weeks 24, 28 and 32—IV nivolumab
Oral SLGN dose weekly for 24 weeks, starting Week 12 through Week 35

### Cohort 2, Group B:

Baseline/Day 1, Weeks 4, 8, 12, 16 and 20—IV nivolumab
Oral SLGN dose weekly for 24 weeks, starting Day 1 through Week 23

### Cohort 3:

Baseline/Day 1, and Weeks 4, 8, 12, 16, and 20—SC VIR-2218, IV nivolumab
Oral SLGN dose weekly for 24 weeks, starting Day 1 through Week 23

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of treating and/or preventing a hepatitis B viral infection in a subject in need thereof, comprising administering to the subject a therapeutically effective amount of a combination therapy regimen comprising
a compound of Formula (I): 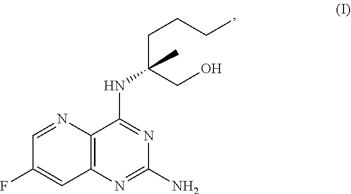
or a pharmaceutically acceptable salt thereof,
a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and
a PD-1/PD-L1 inhibitor, or a pharmaceutically acceptable salt thereof, thereby treating and/or preventing the hepatitis B viral infection in the subject.

2. The method of claim 1, comprising treating the hepatitis B viral infection in the subject in need thereof.

3. The method of claim 1, comprising preventing the hepatitis B viral infection in the subject in need thereof.

4. The method of any one of claims 1 to 3, wherein the compound of Formula (I) has the structure:

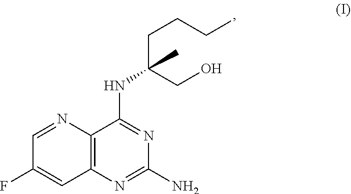

5. The method of any one of claims 1 to 4, wherein the PD-1/PD-L1 inhibitor is nivolumab, pembrolizumab, pidilzumab, BGB-108, SHR-1210, PDR-001, PF-06801591, IBI-308, GB-226, STI-1110, or mDX-400, or a pharmaceutically acceptable salt thereof.

6. The method of claim 5, wherein the PD-1/PD-L1 inhibitor is nivolumab.

7. The method of any one of claims 1 to 3, wherein the PD-1/PD-L1 inhibitor is GS-4224, atezolizumab, avelumab, zimberelimab, AMP-224, MEDI-0680, RG-7446, GX-P2, durvalumab, KY-1003, KD-033, MSB-0010718C, TSR-042, ALN-PDL, STI-A1014, CX-072, or BMS-936559, or a pharmaceutically acceptable salt thereof.

8. The method of any one of claims 1 to 3, wherein the PD-1/PD-L1 inhibitor is: 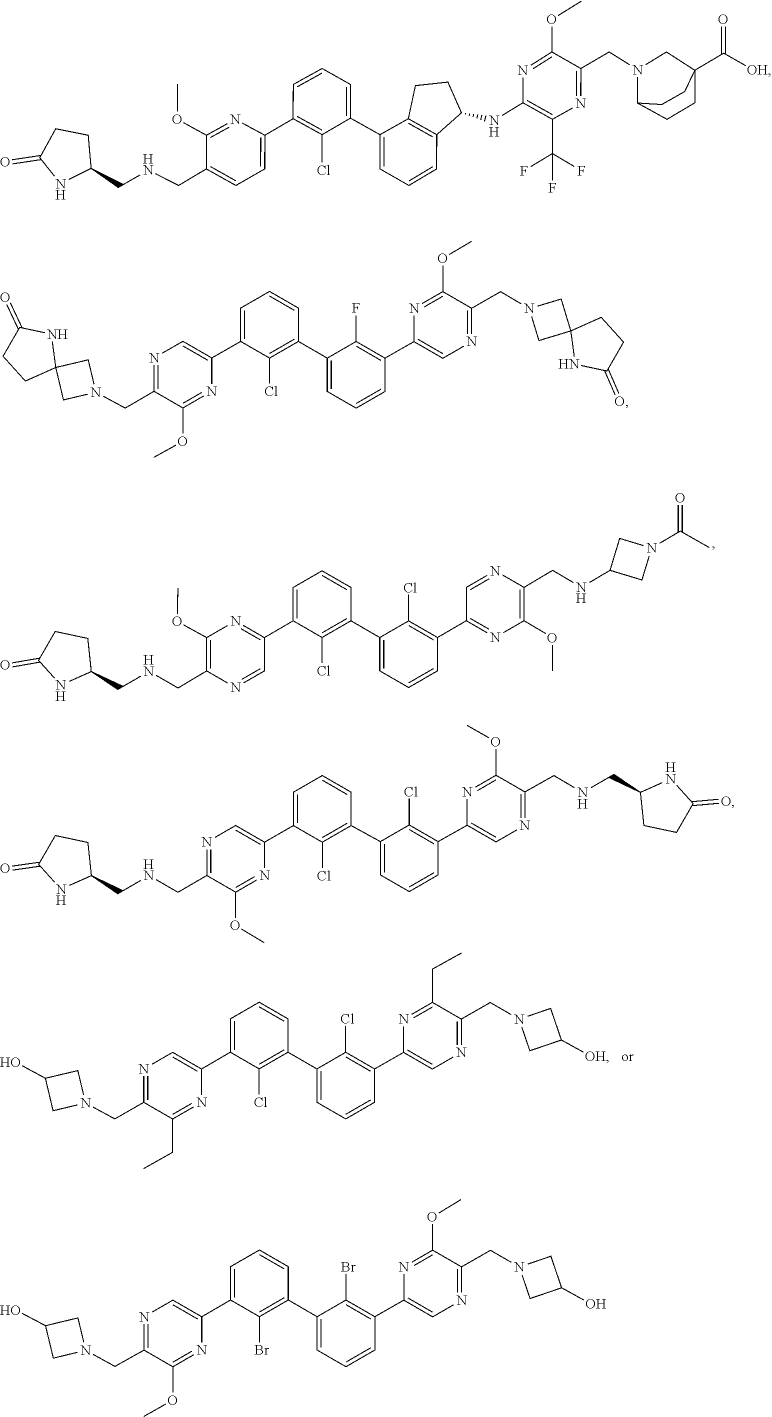
or a pharmaceutically acceptable salt thereof.

9. The method of any one of claims 1 to 8, wherein the compound of Formula (I) is administered once a week for 48 weeks.

10. The method of any one of claims 1 to 8, wherein the compound of Formula (I) is administered once a week for 24 weeks.

11. The method of any one of claims 1 to 9, wherein the compound of Formula (I) is administered orally once a week for 24 weeks.

12. The method of any one of claims 1 to 11, wherein the dsRNA is administered once every 4 weeks for 48 weeks.

13. The method of any one of claims 1 to 11, wherein the dsRNA is administered once every 12 weeks for 48 weeks.

14. The method of any one of claims 1 to 11, wherein the dsRNA is administered once every 12 weeks for 24 weeks.

15. The method of any one of claims 1 to 11, wherein the dsRNA is administered once every 4 weeks for 24 weeks.

16. The method of any one of claims 1 to 15, wherein the dsRNA is administered by subcutaneous injection once every 4 weeks for 24 weeks.

17. The method of any one of claims 6 or 9 to 16, wherein the nivolumab is administered once every 4 weeks for 48 weeks.

18. The method of any one of claims 6 or 9 to 16, wherein the nivolumab is administered once every 12 weeks for 48 weeks.

19. The method of any one of claims 6 or 9 to 16, wherein the nivolumab is administered once every 12 weeks for 24 weeks.

20. The method of any one of claims 6 or 9 to 16, wherein the nivolumab is administered once every 4 weeks for 24 weeks.

21. The method of any one of claims 6 or 9 to 20, wherein the nivolumab is administered by intravenous injection once every 4 weeks for 24 weeks.

22. The method of any one of claims 6 or 9 to 20, wherein the nivolumab is administered by subcutaneous injection once every 4 weeks for 24 weeks.

23. The method of any one of claims 1 to 6 or 9 to 21, wherein the method comprises administering the compound of Formula I, the dsRNA, and nivolumab.

24. The method of claim 23, wherein the dsRNA is administered by subcutaneous injection once every 4 weeks for 24 weeks starting at day 1.

25. The method of claim 23 or 24, wherein the compound of Formula I is administered while the subject is fasting.

26. The method of any one of claims 23 to 25, wherein the compound of Formula I is administered orally once a week for 48 weeks starting at day 1 while the subject is fasting.

27. The method of any one of claims 23 to 26, wherein the nivolumab is administered by intravenous injection once every 4 weeks for 24 weeks starting at day 1.

28. The method of any one of claims 23 to 27, wherein
the compound of Formula I is administered orally once a week for 24 weeks starting at day 1 while the subject is fasting,
the dsRNA is administered by subcutaneous injection once every 4 weeks for 24 weeks starting at day 1, and
the nivolumab is administered by intravenous injection once every 4 weeks for 24 weeks starting at day 1.

29. The method of any one of claims 23 to 25, wherein the compound of Formula I is administered orally once a week for 24 weeks starting at week 12 while the subject is fasting.

30. The method of any one of claims 23 to 25 or 29, wherein the nivolumab is administered by intravenous injection every 4 weeks for 24 weeks starting at week 12.

31. The method of any one of claims 23 to 25, 29 or 30, wherein
the compound of Formula I is administered orally once a week for 24 weeks starting at week 12 while the subject is fasting,
the dsRNA is administered by subcutaneous injection once every 4 weeks for 24 weeks starting at day 1, and
the nivolumab is administered by intravenous injection every 4 weeks for 24 weeks starting at week 12.

32. The method of any one of claims 1 to 20, wherein the method further comprises administering to the subject a compound of Formula (II): 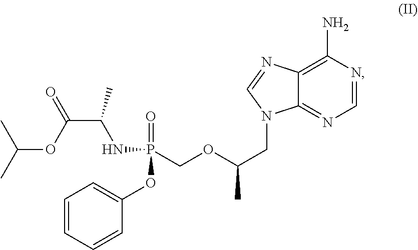 [SMILES: CC(C)OC(=O)[C@H](C)N[P@](=O)(CO[C@H](C)Cn1cnc2c(N)ncnc21)Oc1ccccc1]
or a pharmaceutically acceptable salt thereof.

33. The method of claim 32, wherein the compound of Formula II has the structure:

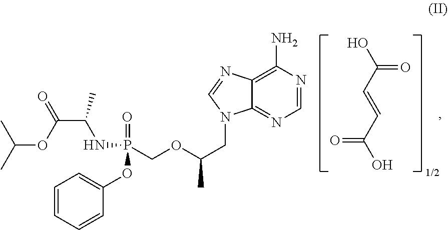

34. The method of claim 32 or 33, wherein the compound of Formula II is administered orally.

35. The method of any one of claims 32 to 34, wherein the compound of Formula II is administered once daily for 48 weeks starting at day 1.

36. The method of any one of claims 32 to 34, wherein administration of the compound of Formula II is terminated if after 36 weeks the subject is characterized by:
(i) a hepatitis B viral load of less than about 20 international units per milliliter (IU/mL);
(ii) negative for the hepatitis B e-antigen (HBeAg); and
(iii) a hepatitis B surface antigen (HBsAg) concentration of less than about 100 international units per milliliter (IU/mL).

37. The method of any one of claims 32 to 34, wherein the compound of Formula II is administered once daily for 36 weeks starting at day 1.

38. The method of any one of claims 32 to 37, wherein the method comprises administering the compound of Formula II, the compound of Formula I, the dsRNA, and nivolumab.

39. The method of any one of claims 32 to 38, wherein the compound of Formula II is administered orally once daily for 36 weeks starting at day 1.

40. The method of claim 38 or 39, wherein the dsRNA is administered by subcutaneous injection once every 4 weeks for 24 weeks starting at day 1.

41. The method of any one of claims 38 to 40, wherein the compound of Formula I is administered orally once a week for 24 weeks starting at week 12 while the subject is fasting.

42. The method of any one of claims 38 to 41, wherein the nivolumab is administered by intravenous injection every 4 weeks for 24 weeks starting at week 12.

43. The method of any one of claims 38 to 42, wherein
the compound of Formula II is administered orally once daily for 36 weeks starting at day 1,
the dsRNA is administered by subcutaneous injection once every 4 weeks for 24 weeks starting at day 1,
the compound of Formula I is administered orally once a week for 24 weeks starting at week 12 while the subject is fasting, and
the nivolumab is administered by intravenous injection every 4 weeks for 24 weeks starting at week 12.

44. The method of any one of claims 1 to 43, wherein the compound of Formula I is administered to the subject in an amount of 1 to 10 mg.

45. The method of any one of claims 1 to 44, wherein the compound of Formula I is administered to the subject in an amount of about 3 mg.

46. The method of any one of claims 1 to 45, wherein the compound of Formula I is administered to the subject in two 1.5 mg doses.

47. The method of any one of claims 1 to 46, wherein the dsRNA is administered to the subject in an amount of 100 to 300 mg.

48. The method of any one of claims 1 to 47, wherein the dsRNA is administered to the subject in an amount of about 200 mg.

49. The method of any one of claims 6 and 9 to 48, wherein the nivolumab is administered to the subject in an amount of 0.1 to 1 mg/kg.

50. The method of claim 49, wherein the nivolumab is administered to the subject in an amount of about 0.3 mg/kg.

51. The method of any one of claims 32 to 43, wherein the compound of Formula II is administered to the subject in an amount of 10 to 50 mg.

52. The method of claim 51, wherein the compound of Formula II is administered to the subject in an amount of about 25 mg.

53. The method of claim 51, wherein the compound of Formula II is administered to the subject in an amount of about 28 mg.

54. The method of any one of claims 1 to 53, wherein the subject has a hepatitis B viral load of less than about 200 international units per milliliter (IU/mL) following completion of treatment.

55. The method of any one of claims 1 to 54, wherein the subject has a hepatitis B viral load of less than about 100 international units per milliliter (IU/mL) following completion of treatment.

56. The method of any one of claims 1 to 55, wherein the subject has a hepatitis B viral load of less than about 20 international units per milliliter (IU/mL) following completion of treatment.

57. The method of any one of claims 1 to 56, wherein the subject has a hepatitis B surface antigen (HBsAg) concentration of less than about 100 international units per milliliter (IU/mL) following completion of treatment.

58. The method of any one of claims 1 to 57, wherein the subject is negative for hepatitis B surface antigen (HBsAg).

59. The method of any one of claims 1 to 58, wherein the subject is negative for the hepatitis B e-antigen (HBeAg) following completion of treatment.

60. A method for manufacturing a medicament for treating and/or preventing a hepatitis B viral infection in a subject in need thereof, characterized in that a therapeutically effective amount of a combination therapy regimen comprising
a compound of Formula (I): 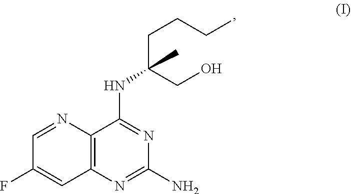
or a pharmaceutically acceptable salt thereof,
a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and
a PD-1/PD-L1 inhibitor, or a pharmaceutically acceptable salt thereof, is used.

61. Use of a therapeutically effective amount of a combination therapy regimen comprising
a compound of Formula (I): 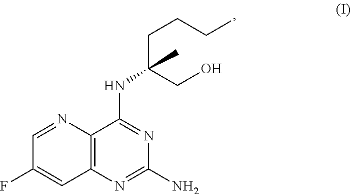
or a pharmaceutically acceptable salt thereof,
a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and
a PD-1/PD-L1 inhibitor, or a pharmaceutically acceptable salt thereof,
for the manufacture of a medicament for the treatment and/or prevention of a hepatitis B viral infection in a subject in need thereof.

62. A combination therapy regimen comprising
a compound of Formula (I): 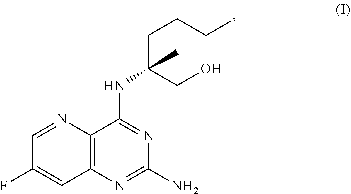
or a pharmaceutically acceptable salt thereof,
a double stranded ribonucleic acid (dsRNA) of SEQ ID NO.:1 and SEQ ID NO.: 2, wherein SEQ ID NO.:1 is 5′-usGfsuga(Agn)gCfGfaaguGfcAfcacsusu-3′ and SEQ ID NO.:2 is 5′-gsusguGfcAfCfUfucgcuucacaL96-3′, and
a PD-1/PD-L1 inhibitor, or a pharmaceutically acceptable salt thereof,
for use in the treatment and/or prevention of a hepatitis B viral infection in a subject in need thereof.
